(19) United States
(12) Patent Application Publication
(10) Pub. No.: US 2026/0240814 A1
(43) Pub. Date: Aug. 20, 2026

(54) Empathogenic Botanical Formulations Comprising Kanna and Their Use

(71) Applicant: KA! Genics Corp., Longmont, CO (US)

(72) Inventors: Alexander Ott, Yonkers, NY (US); Stephanie Wang, Madrid (ES)

(73) Assignee: KA! Genics Corp., Longmont, CO (US)

(21) Appl. No.: 19/472,072

(22) PCT Filed: Apr. 5, 2024
(86) PCT No.: PCT/US2024/023465

§ 371 Date: Oct. 2, 2025

Related U.S. Application Data
(60) Provisional application No. 63/457,370, filed on Apr. 5, 2023.

(51) Int. Cl. A61K 31/404; A61K 36/28; A61K 36/48; A61K 36/53; A61K 36/73; A61K 36/906; A61K 47/26; A61K 47/36; A61K 47/46

(52) U.S. Cl. CPC A61K 31/404; A61K 36/28; A61K 36/48; A61K 36/53; A61K 36/73; A61K 36/906; A61K 47/26; A61K 47/36; A61K 47/46

(57) ABSTRACT

This disclosure relates in some aspects to empathogenic botanical formulations comprising kanna (plant species of the Mesembryanthemum or Sceletium genus), methods of producing such formulations and methods of using such formulations. In some aspects, disclosed formulations produce empathogenic effects in individuals, as well as enhance mood, focus, and energy, and reduce stress and anxiety.

### CROSS-REFERENCE

Priority is claimed under PCT Article 8(1) and Rule 4.10 to U.S. Provisional Application No. 63/457,370 filed Apr. 5, 2023, which is incorporated by reference for all purposes as if fully set forth herein.

### FIELD OF THE INVENTION

This disclosure relates in some aspects to formulations comprising kanna (plant species of the Mesembryanthemum or Sceletium genus). In some aspects, the disclosure relates to methods of producing the formulations and methods of their use. In some aspects, the formulations produce empathogenic effects in individuals, as well as enhance mood, focus, and energy, and reduce stress and anxiety.

### BACKGROUND OF THE INVENTION

Kanna (Mesembryanthemum tortuosum or Sceletium tortuosum) is a succulent plant native to South Africa that has a long history of traditional therapeutic use. Recent studies have also shown that kanna and kanna extracts may be useful in improving mood, and for the treatment of anxiety and depression, as well as other psychological and psychiatric disorders. However, crude plant extracts and plant material consumed according to traditional methods have a characteristically unpleasant bitter taste and may cause nausea. Moreover, the potency of raw plant material and crude extracts is often variable or unknown. The growing interest in both individual and therapeutic uses of kanna have increased the unmet need for kanna formulations that have a measured, standardized potency, and are palatable and enjoyable for consumers.

Provided herein are formulations to meet these needs and others, and that have such advantages and improvements as will become apparent through the disclosure below.

### INCORPORATION BY REFERENCE

Each cited patent, publication, and non-patent literature is incorporated by reference in its entirety, as if each was incorporated by reference individually, and as if each is fully set forth herein. However, no citation should be construed as an admission that a cited reference comes from an area that is analogous or directly applicable to the invention, nor should any citation be construed as an admission that a document or underlying information, in any jurisdiction, is prior art or part of the common general knowledge in the art.

### BRIEF SUMMARY OF THE INVENTION

The following presents a simplified summary of some embodiments of the invention in order to provide a basic understanding of the invention. This summary is not an extensive overview of the invention, and is not intended to identify key or critical elements of the invention or to delineate its full scope. Its sole purpose is to present some embodiments in a simplified form as a prelude to the detailed description.

In some aspects are provided empathogenic botanical formulations of an empathogenic botanical formulation comprising: (a) a kanna composition comprising: (i) a first kanna extract; (ii) a second kanna extract; and (iii) a kanna plant powder; (b) a synergistic plant composition; and (c) a binding composition.

In some embodiments, the kanna plant powder is an unfermented kanna plant powder, a fermented kanna plant powder, or a mixture thereof. In some embodiments, the kanna plant powder has a particle size of about 75 microns.

In some embodiments, the first kanna extract comprises greater than about 7% (w/w) of mesembrine. In some embodiments, the first kanna extract comprises between about 7% and about 9% (w/w) of mesembrine. In some embodiments, the second kanna extract comprises less than about 5% (w/w) of mesembrine. In some embodiments, the second kanna extract comprises between about 2% and about 3% (w/w) of mesembrine.

Also provided are formulations comprising a first kanna extract and a second kanna extract in a weight ratio of between about 1:1 and about 5:1. In some embodiments, the formulation comprises a first kanna extract and a second kanna extract in a weight ratio of about 2:1.

Also provided are formulations of any of the disclosed embodiments, wherein the weight ratio of the kanna plant powder and the total weight of the first and second kanna extracts is between about 1:1 and about 1:5. In some embodiments, the weight ratio of the kanna plant powder and the total weight of the first and second kanna extracts is about 1:3.

In some embodiments, the synergistic plant composition comprises Acacia senegal plant material. In some embodiments, the synergistic plant composition comprises between about 40% and about 80% (v/v) of Acacia senegal plant material. In some embodiments, the Acacia senegal plant material is Acacia senegal plant powder. In some embodiments, the Acacia senegal plant powder has a particle size of about 0.5 mm.

In some embodiments, the synergistic plant composition comprises Acmella oleracea plant material. In some embodiments, the synergistic plant composition comprises between about 5% and about 40% (v/v) of Acmella oleracea plant material. In some embodiments, the Acmella oleracea plant material is Acmella oleracea plant powder. In some embodiments, the Acmella oleracea plant powder has a particle size of about 100 microns.

In some embodiments, the synergistic plant composition comprises Saussurea involucrata plant material. In some embodiments, the synergistic plant composition comprises between about 1% and about 3% (v/v) of Saussurea involucrata plant material.

In some embodiments, the Saussurea involucrata plant material is Saussurea involucrata plant powder. In some embodiments, the Saussurea involucrata plant powder has a particle size of about 1 mm.

In some embodiments, the synergistic plant composition comprises Lavandula angustifolia plant material. In some embodiments, the synergistic plant composition comprises between about 0.1% and about 5% (v/v) of Lavandula angustifolia plant material. In some embodiments, the Lavandula angustifolia plant material is Lavandula angustifolia plant powder. In some embodiments, the Lavandula angustifolia plant powder has a particle size of about 100 microns.

In some embodiments, the synergistic plant composition comprises Mentha spicata plant material. In some embodiments, the Mentha spicata plant material is Mentha spicata extract. In some embodiments, the synergistic plant composition comprises between about 1% and about 3% (v/v) of Mentha spicata extract.

In some embodiments, the synergistic plant composition comprises Zingiber officinale plant material.

In some embodiments, the synergistic plant composition comprises Rosa sp. plant material.

Also provided are synergistic plant compositions comprising a combination of plant materials selected from the group of plant materials consisting of Acacia senegal plant material, Acmella oleracea plant material, Saussurea involucrata plant material, Lavandula angustifolia plant material, Mentha spicata extract, Zingiber officinale plant material, and Rosa sp. plant material. In some embodiments, the combination of plant materials comprises two or more plant materials selected from said group of plant materials. In some embodiments, the combination of plant materials comprises three or more plant materials selected from said group of plant materials. In some embodiments, the combination of plant materials comprises four or more plant materials selected from said group of plant materials. In some embodiments, the combination of plant materials comprises five or more plant materials selected from said group of plant materials. In some embodiments, the combination of plant materials comprises six or more plant materials selected from said group of plant materials.

In some embodiments, the synergistic plant composition comprises: Acacia senegal plant material, Acmella oleracea plant material, Saussurea involucrata plant material, Lavandula angustifolia plant material, Mentha spicata extract, Zingiber officinale plant material, and Rosa sp. plant material.

In some embodiments, the binding composition comprises guar gum. In some embodiments, the binding composition comprises vegetable glycerin. In some embodiments, the binding composition comprises distilled water. In some embodiments, the binding composition comprises distilled water and vegetable glycerin in a weight ratio of between about 1:1 and about 20:1. In some embodiments, the water:glycerin weight ratio is between about 4:1 and about 7:1. In some embodiments, the binding composition comprises guar gum, vegetable glycerin, and distilled water.

In some embodiments, the formulation comprises a flavoring agent. In embodiments, the flavoring agent is an organic or natural flavor, a sweet flavor, a fruit flavor, a berry flavor, or a combination thereof.

In some embodiments, the formulation comprises a coloring agent. In some embodiments, the coloring agent is a vegetable extract. In embodiments, the vegetable extract comprises an extract of any of spinach, blueberry, carrot, beet, red cabbage, turmeric, annatto seed, saffron, paprika, tomato, grape, cherry, black carrot, pumpkin, sweet potato, elderberry, spirulina, bell pepper, sweet potato, kale, hibiscus flower, matcha, parsley, mint, pea, cucumber, aronia, blackberry, raspberry, cranberry, and a combination thereof.

In some other aspects are provided empathogenic botanical formulations, comprising: (a) a kanna composition comprising: (i) a first kanna extract; (ii) a second kanna extract; and (iii) a kanna plant powder; (b) a synergistic plant composition; (c) a binding composition; (d) at least one flavoring agent; and (e) at least one coloring agent.

In some other aspects are provided empathogenic botanical formulations, comprising: (a) a kanna composition comprising: (i) a first kanna extract; (ii) a second kanna extract; and (iii) a kanna plant powder; (b) a synergistic plant composition comprising: (i) Acacia senegal plant powder; (ii) Acmella oleracea plant powder; and (iii) any one or more of Acacia senegal plant material, Acmella oleracea plant material, Saussurea involucrata plant material, Lavandula angustifolia plant material, Mentha spicata extract, Zingiber officinale plant material, and Rosa sp. plant material.

In some other aspects are provided empathogenic botanical formulations, comprising: (a) a kanna composition comprising: (i) a first kanna extract; (ii) a second kanna extract; and (iii) a kanna plant powder; (b) a synergistic plant composition; and (c) a binding composition comprising: (i) guar gum; (ii) vegetable glycerin; and (iii) distilled water.

In some other aspects are provided empathogenic botanical formulations, comprising: (a) a kanna composition comprising: (i) a first kanna extract; (ii) a second kanna extract; and (iii) a kanna plant powder; (b) a synergistic plant composition comprising: (i) Acacia senegal plant powder; (ii) Acmella oleracea plant powder; and (iii) any one or more of Acacia senegal plant material, Acmella oleracea plant material, Saussurea involucrata plant material, Lavandula angustifolia plant material, Mentha spicata extract, Zingiber officinale plant material, and Rosa sp. plant material; and (c) a binding composition comprising: (i) guar gum; (ii) vegetable glycerin; and (iii) distilled water.

In some embodiments, the formulation does not contain an added preservative. In some embodiments, the formulation does not contain added sugar. In some embodiments, the formulation does not contain an artificial sweetener. In some embodiments, the formulation does not contain a synthetic polymer. In some embodiments, the formulation does not contain an added antioxidant. In some embodiments, the formulation does not comprise agar.

In some embodiments, the formulation is in unit dosage form.

In some embodiments, the formulation comprises at least one non-naturally occurring substance.

In some other aspects are provided empathogenic botanical formulations, comprising: (a) a kanna composition; (b) a synergistic plant composition; and (c) a binding composition.

Also provided is a method of manufacturing an empathogenic botanical formulation comprising: (a) preparing a powder blend of the kanna composition, the synergistic plant composition, and the binding composition; (b) mixing the powder blend with a liquid to obtain a paste; and (c) drying the paste to obtain the empathogenic botanical formulation.

In some embodiments, the liquid comprises distilled water. In some embodiments, the liquid comprises vegetable glycerin. In some embodiments, the liquid comprises distilled water and vegetable glycerin. In some embodiments, the liquid comprises distilled water and vegetable glycerin in a water:glycerin weight ratio of between about 1:1 and about 20:1. In some embodiments, the liquid comprises distilled water and vegetable glycerin in a water:glycerin weight ratio of between about 4:1 and about 7:1.

In some embodiments, the method further comprises dividing the paste into unit doses prior to drying the paste. In some embodiments, the method does not comprise heating the powder blend, the paste, or the empathogenic botanical formulation. In some embodiments, each step of the method is conducted at ambient temperature.

In some other aspects are provided formulations used in modulating neurotransmission in an individual. Also provided is the use of the formulation of any one of disclosed embodiments for modulating neurotransmission in an individual. Also provided is a method of modulating neurotransmission in an individual, comprising administering to an individual an empathogenic botanical formulation disclosed herein.

In some embodiments, the modulated neurotransmission is serotonergic neurotransmission. In some embodiments modulating serotonergic neurotransmission comprises any one or more of: activating a serotonin receptor, stimulating release of serotonin, and reducing serotonin uptake by inhibiting the function of a serotonin transporter (SERT).

In some embodiments, the neurotransmission is GABAergic neurotransmission.

In some embodiments, modulating neurotransmission comprises inhibiting phosphodiesterase 4.

In some embodiments, modulating neurotransmission results in an empathogenic effect. Also provided are empathogenic botanical formulations for use in producing an empathogenic effect in an individual. Also provided is the use of an empathogenic botanical formulation for producing an empathogenic effect in an individual. In some embodiments, a method of producing an empathogenic effect in an individual comprises administering to the individual the formulation of any of the disclosed embodiments.

In some embodiments, the empathogenic effect comprises any one or more of reducing stress, reducing anxiety, improving mood, increasing energy, reducing fatigue, creating a feeling of connection with self and others, and improving general mental health and well-being; or comprises feelings of any one or more of: deeper connection with oneself and others; restoration and coherence of body, heart, mind and spirit; head-heart harmony; being in balance and healthy homeostasis; joy and the highest form of well being; the fullness of one's aliveness; radiating from within; the ability to positively affect everything around oneself; elevated vibes; and aliveness and lifted mood.

In some embodiments, modulating neurotransmission treats a medical condition. Also provided is a formulation or any of the disclosed embodiments for use in treating a medical condition in a subject in need of such treatment. Also provided is the use of the formulation of any of the disclosed embodiments in the manufacture of a medicament for treating a medical condition.

Also provided is a method of treating a medical condition in a subject in need of such treatment, comprising administering to the subject the formulation of any of the disclosed embodiments.

In some embodiments, the medical condition is a mental health disorder. In some embodiments, the mental health disorder is selected from the group consisting of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

In some embodiments, the mental health disorder is anxiety, depression, addiction, an eating disorder, an alcohol or drug abuse or dependence disorder, OCD, or PTSD. In some embodiments, depression is major depressive disorder (MDD) or treatment-resistant depression (TRD). In some embodiments, anxiety is generalized anxiety disorder (GAD) or menopause-related anxiety.

In some embodiments, the formulation is administered together with one or more therapeutically beneficial activities. In some embodiments, the formulation is administered together with one or more sessions of psychotherapy.

Also provided is a formulation for use in modulating neurotransmission in an individual. Also provided is the use of a formulation of any of the disclosed embodiments for modulating neurotransmission in an individual. In some embodiments, a method of modulating neurotransmission in an individual, comprises administering to the individual the formulation of any of the disclosed embodiments. Also provided are formulations manufactured according to the method of any of the disclosed embodiments, for use in modulating neurotransmission in an individual. Also provided is the use of a formulation manufactured according to the method of any of the disclosed embodiments for modulating neurotransmission in an individual. In some embodiments, the method of modulating neurotransmission comprises administering to the individual the formulation manufactured according to the method of any of the disclosed embodiments.

Also provided is a formulation for use in producing an empathogenic effect in an individual. Also provided is the use of the formulation of any of the disclosed embodiments for producing an empathogenic effect in an individual. Also provided is a method of producing an empathogenic effect in an individual, comprising administering to the individual the formulation of any of the disclosed embodiments. Also provided is a formulation manufactured according to the method of any of the disclosed embodiments, for use in producing an empathogenic effect in an individual. Also provided is the use of the formulation manufactured according to the method of any of the disclosed embodiments for producing an empathogenic effect in an individual. In some embodiments, the method of producing an empathogenic effect in an individual, comprises administering to the individual the formulation manufactured according to the method of any one of the disclosed embodiments.

Also provided is a formulation of any of the disclosed embodiments for use in treating a medical condition in a subject in need of such treatment. Also provided is the use of the formulation of any of the disclosed embodiments in the manufacture of a medicament for treating a medical condition. Also provided is a method of treating a medical condition in a subject in need of such treatment, comprising administering to the subject the formulation of any of the disclosed embodiments. Also provided is a formulation manufactured according to the method of any of the disclosed embodiments, for use in treating a medical condition in a subject in need of such treatment. Also provided is the use of the formulation manufactured according to the method of any of the disclosed embodiments in the manufacture of a medicament for treating a medical condition. In some embodiments, a method of treating a medical condition in a subject in need of such treatment, comprises administering to the subject the formulation manufactured according to the method of any of the disclosed embodiments.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the invention as set forth in the claims.

The headings within this document are being utilized only to expedite its review by a reader. They should not be construed as limiting the invention in any manner.

### DETAILED DESCRIPTION OF THE INVENTION

While various aspects and features of certain embodiments are summarized above, the following detailed description illustrates several exemplary embodiments in further detail to enable one of skill in the art to practice such embodiments, and to make and use the full scope of the invention claimed. The described examples are provided for illustrative purposes and are not intended to limit the scope of the invention or its applications. It will be understood that many modifications, substitutions, changes, and variations in the described examples, embodiments, applications, and details of the invention illustrated herein can be made by those skilled in the art without departing from the spirit of the invention, or the scope of the invention as described in the appended claims.

The scope of the invention includes all embodiments and formulations thereof, not only those expressly described below, and it will be understood that many modifications, substitutions, changes, and variations in the described embodiments, applications, and details of the invention can be made by those in the art without departing from the spirit of the invention, or the scope of the invention as claimed.

### A. GENERAL DEFINITIONS AND TERMS

As used in this specification and the appended claims, the singular forms “a,” “an,” and “the” include plural referents unless the context clearly dictates otherwise. Thus, for example, reference to “an active agent” includes reference to a combination of two or more active agents, and reference to “an excipient” includes reference to a combination of two or more excipients. While the term “one or more” may be used, its absence (or its replacement by the singular) does not signify the singular only, but simply underscores the possibility of multiple agents or ingredients in particular embodiments.

The terms “comprising,” “including,” “such as,” and “having” are intended to be inclusive and not exclusive (i.e., there may be other elements in addition to the recited elements, or the recited elements may be exemplary only). Thus, the term “including” is used to mean, and is used interchangeably with, the phrase “including but not limited to,” as are similar terms like “such as.” The term “or” is used to mean, and is used interchangeably with, the term “and/or” unless context clearly indicates otherwise.

Unless otherwise indicated, all numbers expressing quantities of ingredients, properties such as concentration, reaction conditions, and so forth, used to describe and claim certain embodiments of the invention are to be understood as being modified in some instances by the term “about.” Accordingly, in some embodiments (equivalently, and simply for shorthand, “in embodiments”), the numerical parameters herein are approximations that can vary depending upon, e.g., the desired properties sought to be obtained by an embodiment. In embodiments, “about” refers to plus or minus five percent (±10%) of the recited unit of measure. In embodiments, “about” refers to plus or minus five percent (±5%) of the recited unit of measure.

The term “substantially,” where it is applied to modify a feature or limitation herein, will be read in the context of the invention and in light of the knowledge in the art to provide the appropriate certainty, e.g., by using a standard that is recognized in the art for measuring the meaning of “substantially” as a term of degree, or by ascertaining the scope as would one of skill in the art.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments may contain certain errors necessarily resulting from the standard deviation found in their respective testing measurements.

Herein, “an effective amount” refers to an amount of active agent(s) that is non-toxic and sufficient to provide the desired effect with performance at a reasonable benefit/risk ratio attending any similar use of a like agent. The effective amount will vary depending upon the subject and the effect sought, the manner of administration, and the like, all of which can readily be determined by one of skill.

Herein, “effect” or “efficacy” means the responses(s) in a mammal, and preferably a human, after administration by a disclosed combination or with a disclosed method that are judged to be desirable and beneficial. Depending on the effect or improvement sought, and depending on the particular agent(s) in the disclosed combination or method, those responses may differ, but will be readily understood by those of skill.

Unless defined otherwise, all technical and scientific terms herein have the meaning as commonly understood by one having ordinary skill in the art to which this invention belongs, who as a shorthand may be referred to simply as “one of skill.” Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only and is not intended to be limiting.

Generally, the nomenclature used and procedures performed herein are those known in fields relating to one or more aspects of the invention, such as botany, natural products extraction, natural products formulation, food science, chemistry including flavor chemistry, biology, pharmacology, and medicine, and are those that will be well known and commonly employed in such fields. Standard techniques and procedures will be those generally performed according to conventional methods in the art.

Still additional definitions and abbreviations are provided elsewhere herein.

### B. EMPATHOGENIC BOTANICAL FORMULATIONS

In one aspect, provided is an empathogenic botanical formulation, comprising: (i) a kanna composition; (ii) a synergistic plant composition; and (iii) a binding composition.

In some embodiments, the compositions are nutraceutical compositions. In some embodiments, the compositions are pharmaceutical compositions. Pharmaceutical compositions are compositions that include the compounds together in an amount (for example, in a unit dosage form) with a pharmaceutically acceptable carrier, diluent, or excipient. It should be understood that some embodiments do not have a single carrier, diluent, or excipient alone, but include multiple carriers, diluents, and/or excipients.

Although disclosed formulations may in some embodiments be referred to as “pharmaceutical” compositions or for “pharmaceutical” purpose or preparation, it will be appreciated that the term simply means that a formulation is contemplated or shown to possess therapeutic or beneficial effects when administered for its intended purpose to a mammal, such as a human. It therefore will be understood that the disclosed formulations are useful regardless of the regulatory regime under which they are ultimately sold (e.g., as prescription pharmaceutical drug products or non-prescription over-the-counter (OTC) drug products, as nutritional supplements or “nutraceuticals,” as food or dietary supplements, or as “functional food” products which beneficially affect one or more target functions in the body, beyond adequate nutritional effects, in a way that is relevant to either an improved state of health and wellbeing and/or reduction in risk of disease), and also if not sold under a specific regulatory regime at all, whether in the U.S. or any other country. It also will be understood that the disclosed compositions and formulations may be considered to have or to provide therapeutic or beneficial effects whether or not they are marketed in any jurisdiction (or capable of being marketed in any jurisdiction, under any applicable regulatory regime) as having such.

For the avoidance of doubt, a formulation may provide a therapeutic or beneficial effect even when not intended to diagnose, treat, cure, or prevent any disease, and when not marketed as being for purposes of diagnosing, treating, curing, or preventing any disease.

In some embodiments, one or more compounds or compositions in a formulation may be Generally Recognized As Safe (GRAS). “GRAS” as used in connection with one or more ingredients means that the ingredients, if used as food additives, have been (or may be, at the appropriate time and/or in the appropriate circumstances) adequately shown to be safe under the conditions of their intended use.

In some embodiments, one or more compounds or compositions in a formulation may be pharmaceutically acceptable. “Pharmaceutically acceptable” as used in connection with one or more ingredients means that the ingredients are generally safe and, within the scope of sound medical judgment, suitable for use in contact with the cells of humans and other animals without undue toxicity, irritation, allergic response, or complication, and commensurate with a reasonable risk/benefit ratio.

In preferred embodiments, the compounds and compositions in a formulation, whether GRAS, pharmaceutically acceptable, or as otherwise defined by regulation or their application, will be safe for human consumption when ingested according to their intended use.

a. Kanna Compositions

Kanna (Mesembryanthemum tortuosum) is a succulent plant in the Aizoaceae family, native to South Africa. It is also frequently referred to as Sceletium tortuosum, among other names (e.g., channa, kougoed). Since prehistoric times, humans have consumed kanna for its anxiolytic, antidepressant, and analgesic properties (Gericke, et al. J. Ethnopharmacol. 2008; 119(3):653-663).

Many alkaloids can be isolated from kanna, including mesembrine, mesembrenone, mesembrenol, and tortuosamine (see Smith, et al. J. Ethnopharmacol. 1996; 50(3):119-130, and figures below). Mesembrine is one of the principal kanna alkaloids, and has been shown to inhibit serotonin reuptake by blocking the activity of the serotonin transporter (SERT) (Harvey, et al. J. Ethnopharmacol. 2011; 137(3):1124-1129).

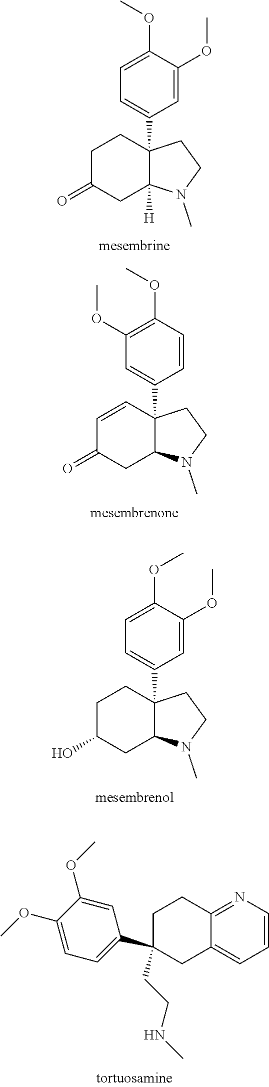 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC ; COc1ccc([C@@]23CC[C@@H](O)C[C@@H]2N(C)CC3)cc1OC ; CNCC[C@]1(c2ccc(OC)c(OC)c2)CCc2ncccc2C1]

In addition to functioning as a SERT inhibitor, mesembrine is shown to directly promote serotonin release. As such, mesembrine is thought to play an important role in the psychoactive and potentially therapeutic effects of kanna. Mesembrine also behaves as a phosphodiesterase 4 (PDE4) inhibitor. Mechanistically, PDE4 inhibitors block the ability of PDE4 to degrade cyclic adenosine monophosphate (CAMP). PDE4 inhibitors are known to possess neuroprotective, anti-inflammatory, and procognitive effects, e.g., improving memory and promoting wakefulness (Barad, et al. PNAS. 1998; 95(25):15020-15025; Lelkes, et al. Pharmacol Biochem Behav. 1998; 60(4):835-839; Block, et al. NeuroReport. 2001; 12(7):1507-1511; Chen, et al. Neurosci. Lett. 2007; 418(2):165-169). PDE4 inhibitors have been investigated for the treatment of many central nervous system (CNS) diseases and disorders, including mental health disorders such as depression and anxiety (Brandon & Rotella. Annu Rep Med Chem. 2007; 42:3-12).

Recent in vivo and clinical case study reports suggest that kanna's ability to modulate serotonergic neurotransmission and PDE4 inhibition may be useful in treating anxiety and depressive disorders (Gericke. Aust J Med Herbalism. 2001; 13:3; Terburg, et al. Neuropsychopharmacol. 2013; 38(13):2708-2716; Harvey, et al. J. Ethnopharmacol. 2011; 137(3):1124-1129).

As used herein, “kanna” refers to the kanna plant (e.g., Mesembryanthemum tortuosum or Sceletium tortuosum), including unprocessed or unrefined plant material, or plant material that has been physically processed (e.g., dehydrated, dried, pulverized, or ground). In addition to Mesembryanthemum tortuosum (Sceletium tortuosum), eight related species have been classified in the same genus: M. crassicaule, M. emarcidum, M. exalatum, M. expansum, M. archeri (S. rigidum), M. ladismithiense (S. strictum), M. tortuosum and M. varians. The Dictionary of Natural Products describes 31 different alkaloidal components from five sources of Sceletium: S. joubertii, S. namaquense (both now considered synonymous with S. tortuosum), S. strictum, S. subvelutium (also now classified as S. tortuosum), and S. tortuosum (id.). The concentrations and ratios of specific alkaloids, such as those shown above among others (e.g., mesembranol, alkaloid A4, chennaine) can vary between species (id.; Gericke and Viljoen, J. Ethnopharmacol. 2008; 119:653-663). All species of kanna described herein and otherwise known in the art are embraced by the term “kanna” and may be used in disclosed formulations.

“Kanna” also refers to extracts produced from kanna plant material, for example, aqueous extracts, alcoholic extracts (e.g., ethanolic extracts), tinctures, and the like. Kanna extracts can be crude, or they can be processed to increase or decrease the concentration of a particular alkaloid or another compound. Kanna extracts can also be purified to remove organic or inorganic impurities, such as by mechanical (e.g., filtration), chemical, or physical means. The present disclosure includes all such extracts.

Kanna and products derived therefrom (e.g., extracts, powders, etc.) may be fermented. Fermentation is a common technique used to reduce the concentration of oxalic acid and oxalates in kanna (Smith, et al. J. Ethnopharmacol. 1996; 50(3):119-130). Kanna and products derived therefrom also may be unfermented. The kanna used in disclosed formulations may be fermented, unfermented, or a mixture.

One or more forms of kanna may be combined with each other in disclosed formulations and kanna compositions. For example, a disclosed formulation or kanna composition may in some embodiments comprise a combination of kanna plant material and kanna extract, or one or more kanna extracts having different physical or chemical characteristics. In some embodiments, wherein a disclosed kanna composition comprises several component forms of kanna (e.g., kanna plant powder and kanna extract) the amounts of the kanna components are selected such that the combination produces an improved empathogenic effect relative to the administration of a single component a alone. For example, in embodiments wherein a disclosed kanna composition comprises both kanna plant powder and kanna extract, an individual who consumes a disclosed empathogenic botanical formulation may feel an initial burst of empathogenic effect and/or euphoria (e.g., effects provided by the kanna extract) and subsequently a prolonged, sustained, “grounded” empathogenic effect and/or elevation in mood (e.g., effects provided by the kanna plant powder).

In some embodiments, disclosed are empathogenic botanical formulations comprising a kanna composition. In embodiments, the kanna composition comprises a kanna plant powder. In embodiments, the kanna plant powder is fermented. In some embodiments, the kanna plant powder is unfermented.

In some embodiments, the kanna plant powder has the following characteristics, as represented by the exemplary certificate of analysis below:

CERTIFICATE OF ANALYSIS

| Product Name | Kanna extract | | |
|---|---|---|---|
| Botanical Name | Sceletium tortuosum | | |
| Batch Number | 20220325 | Manufacture Date | 2022 Mar. 25 |
| Sterilization Method | High temperature sterilization | | |
| Item of Analysis | Specification | Results | Test Methods |
| Extract Ratio | 50:1 | 50:1 | TLC |

Organoleptic

| | | | |
|---|---|---|---|
| Appearance | Powder | Conforms | Organoleptic |
| Color | Brown Yellow Powder | Conforms | Organoleptic |
| Odor | Characteristic | Conforms | CP2010 |
| Taste | Characteristic | Conforms | CP2010 |

Physical Characteristics

| | | | |
|---|---|---|---|
| Particle Size | 100% pass 80 mesh | 80 MESH | CP2010 |
| Loss on drying | 5% NMT (%) | 2.11% | USP<731> |
| Total ash | 5% NMT (%) | 2.45% | USP<561> |
| Bulk Density | 45-60 g/100 mL | 52 g/100 mL | CP2010IA |
| Residue of Solvents | None | Conform | NLS-QCS-1007 |

Heavy Metals

| | | | |
|---|---|---|---|
| Total Heavy Metals | 10 ppm Max | Conform | USP<231>method II |
| Lead (Pb) | 2 ppm NMT | 0.013 ppm | ICP-MS |
| Arsenic (As) | 2 ppm NMT | 0.21 ppm | ICP-MS |
| Cadmium (Cd) | 2 ppm NMT | 0.04 ppm | ICP-MS |
| Mercury (Hg) | 0.2 ppm NMT | 0.005 ppm | ICP-MS |

Microbiological Tests

| | | | |
|---|---|---|---|
| Total Plate Count | 3000 cfu/g Max | Conform | USP<61> |
| Yeast & Mold | 300 cfu/g Max | Conform | USP<61> |
| E. Coli. | Negative | Conform | USP<61> |
| Salmonella | Negative | Conform | USP<61> |
| Staphylococcus | Negative | Conform | USP<61> |

Storage: In tight, light resistant containers, avoid exposure to direct sunlight,
moisture and excessive heat.
Shelf Life: 24 months if stored under the conditions above and stay in original packaging.

In embodiments, the kanna plant powder above may be referred to as a full-spectrum kanna extract, and is one non-limiting example thereof, other examples being known in this disclosure and to those of skill.

In some embodiments, wherein a disclosed empathogenic botanical formulation comprises a kanna plant powder, the kanna plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the kanna plant powder is less than about 200 microns, less than about 150 microns, less than about 125 microns, less than about 100 microns, less than about 75 microns, less than about 50 microns, or less than about 25 microns. In some embodiments, the particle size of the kanna plant powder is less than about 75 microns.

In some embodiments, wherein a disclosed empathogenic botanical formulation comprises a kanna plant powder, the kanna plant powder is present at an amount of between about 0.1% and 5% (v/v) of the total plant material in the formulation (i.e., the sum of the plant material in the kanna composition and the synergistic plant composition). In some embodiments, the kanna plant powder is present at an amount of about 0.1%, 0.5%, 1.0%, 1.25%, 1.5%, 2.5%, or 5%, including ranges in between these values. In certain preferred embodiments, the kanna plant powder is present at an amount of about 1.0%.

In some embodiments, the kanna composition comprises a kanna extract. The kanna extract can be an aqueous extract, or an extract of an organic solvent (e.g., an alcoholic extract). In some embodiments, the kanna extract is an aqueous extract, such as those disclosed in U.S. Pat. No. 10,864,239 (“High yield extraction method for and products of kanna plants”), incorporated by reference as if fully disclosed herein.

In one exemplary extraction process, a kanna extract is obtained by harvesting and drying kanna plant material; mixing the plant material with a solvent to extract alkaloids from the plant material; filtering the solvent-plant mixture to remove the solvent (containing extracted alkaloids) from the plant material; drying the plant material; purifying and separating the extracted alkaloids from the solvent; and mixing the purified alkaloids back into the dried plant material.

In this exemplary extraction process, the solvent may be a known food grade solvent, including but not limited to ethanol, methanol, hydroalcohol, acetone, acetonitrile, hexane, heptane, hexane, chloroform, dichloromethane, water, and mixtures thereof. For instance, in one embodiment, the solvent is ethanol. In some embodiments, the solvent-plant mixture is heated to facilitate extraction of the kanna alkaloids into the solvent. Filtration can be accomplished using any suitable means known to one of skill. After filtration, the solvent may be removed to concentrate the extracted kanna alkaloids. Removal of the solvent can be accomplished, for example, by distillation, open-dish evaporation, reduced-pressure evaporation, rotary evaporation, vacuum, lyophilization, or a combination of methods thereof. After filtration and optional concentration, the alkaloid extract may be subjected to purification and separation techniques to isolate the alkaloids from other undesired phytochemicals extracted by the solvent. Such purification and separation can be carried out by column chromatography comprising a reverse stationary phase, a normal stationary phase or combination thereof, ion-exchange chromatography, gel-permeation (molecular sieve) chromatography, affinity chromatography, paper chromatography, thin-layer chromatography, gas chromatography, dye-ligand chromatography, hydrophobic interaction chromatography, pseudoaffinity chromatography, and/or high-pressure liquid chromatography (HPLC). Once purified and separated, the isolated alkaloid products may be washed, filtered and dried. The purified, concentrated products (e.g., mesembrine and other target alkaloids and/or optionally other phytochemicals) can then be recombined with the dried kanna plant material to produce a kanna plant material enriched in kanna alkaloids.

In some embodiments, the kanna extract comprises a mixture of a plant component (e.g., kanna plant powder) and an extract (e.g., an aqueous or organic extract). In some embodiments, the kanna extract comprises a plant portion and an extract portion in a plant:extract ratio of between about 5:1 and 100:1 (on a weight or volume basis). In some embodiments, the plant:extract ratio is between about 5:1 and 10:1, including 5:1, 6:1, 7:1, 8:1, 9:1, 10:1, and values in between these ratios. In some embodiments, the kanna extract comprises a plant portion and an extract portion in a plant:extract ratio of between about 6:1 and 7:1. In some embodiments, the plant:extract ratio is between about 10:1 and 30:1, including about 10:1, 15:1, 20:1, 25:1, 30:1, and values in between these ratios. In some embodiments, the kanna extract comprises a plant portion and an extract portion in a plant:extract ratio of about 20:1. In some embodiments, the plant:extract ratio is between about 40:1 and 60:1, including about 40:1, 45:1, 50:1, 55:1, 60:1, and values in between these ratios. In some embodiments, the kanna extract comprises a plant portion and an extract portion in a plant:extract ratio of about 50:1.

In some embodiments, the kanna extract is an extract with a mesembrine content of between about 1% and 20% (v/v) of the total extract material (i.e., the sum of the mesembrine content in the kanna extract). In some embodiments the mesembrine is present in the kanna extract at an amount between 1% and 10%, including 1%, 2%, 3%, 4%, 5%, 6%, 7%, 8%, 9%, 10%, and values between these amounts.

In some embodiments, the mesembrine content is present at an amount between 2% and 3% of the kanna extract, including 2.1%, 2.2%, 2.3%, 2.4%, 2.5%, 2.6%, 2.7%, 2.8%, 2.9%, 3.0%, and ranges between these values.

In some embodiments, the kanna extract is an extract with a mesembrine content between about 7.5% and 8.5%, including 7.5%, 7.6%, 7.7%, 7.9%, 8.0%, 8.1%, 8.2%, 8.3%, 8.4%, 8.5%, and ranges between values.

In some embodiments, the kanna extract is an extract with a mesembrenone content of between about 1% and 20% (v/v) of the total extract material (i.e., the sum of the mesembrenone content in the kanna extract). In some embodiments the mesembrenone is present in the kanna extract at an amount between 1% and 10%, including 1%, 2%, 3%, 4%, 5%, 6%, 7%, 8%, 9%, 10%, and values between these amounts. In some embodiments, the kanna extract is an extract with a mesembrenone content of less than about 1%.

In some embodiments, the kanna extract is an extract with a mesembrenol content of between about 1% and 20% (v/v) of the total extract material (i.e., the sum of the mesembrenol content in the kanna extract). In some embodiments the mesembrenol is present in the kanna extract at an amount between 1% and 10%, including 1%, 2%, 3%, 4%, 5%, 6%, 7%, 8%, 9%, 10%, and values between these amounts. In some embodiments, the kanna extract is an extract with a mesembrenol content of less than about 1%.

In some embodiments, the kanna extract is an extract with a tortuosamine content of between about 1% and 20% (v/v) of the total extract material (i.e., the sum of the tortuosamine content in the kanna extract). In some embodiments the tortuosamine is present in the kanna extract at an amount between 1% and 10%, including 1%, 2%, 3%, 4%, 5%, 6%, 7%, 8%, 9%, 10%, and values between these amounts. In some embodiments, the kanna extract is an extract with a tortuosamine content of less than about 1%.

In some embodiments, the kanna extract has the following characteristics, as represented by the exemplary certificate of analysis below:

Typical Certificate of Analysis

| Product: | SCELETIUM TORTUOSUM EXTRACT | | |
|---|---|---|---|
| Botanical Origin: | Sceletium tortuosum | | |
| Production Date: | 29 May 2023 | | |
| Expiration Date: | 29 May 2025 | | |
| Lot Number: | 2674/0523 | | |
| Country of Origin: | South Africa | | |
| Harvesting protocol: | In accordance with the conservation control authority of South Africa | | |
| PARAMETER | SPECIFICATION | METHOD | RESULT |
| Plant Part Used: | Whole Herba | Visual | Whole Herba/Pass |
| Extraction Method: | Ethanolic Extraction | Proprietary | Verified on BMR |
| Plant/Extract Ratio: | 6-7:1 [DER] | S/L | Verified on BMR |
| Product Characterization: | As per in-house Ref. No. DV-17 | GC-MS | Verified/No. DV-17 |
| Appearance: | Fine powder | Visual | Powder/Pass |
| Colour: | Light Tan to Beige | Visual | Beige/Pass |
| Odour: | Characteristic | Organoleptic | Characteristic/Pass |
| Solubility: | 16 g/Lt in H2O @ 20* C. (±10%) | Physical | 16.1 g/Lt/Pass |
| Main Active Ingredient: | 2.4-2.6% Mesembrine | HPLC | 2.6%/Pass |
| Main Active Ingredient: | Mannitol | 3rd Party | Present as per BMR |
| Particle Size: | 60-100 micron | Sieve | Pass/Complies |
| Moisture: | MNT 5% | AOAC 950.46 | 1.9%/Pass |

| | |
|---|---|
| Storage and Handling: | Closed, sealed storage container. |
| | Cool and dry conditions - (20-25* C. @ 65% RH) |
| | This product is non-GMO |

In some embodiments, the kanna extract has the following characteristics, as represented by the exemplary certificate of analysis below:

Typical Certificate of Analysis

| Product: | SCELETIUM TORTUOSUM EXTRACT | | |
|---|---|---|---|
| CAS Number: | Not listed | | |
| IUPAC Number: | Not listed | | |
| Botanical Origin: | Sceletium tortuosum | | |
| Production Date: | 6 Feb. 2023 | | |
| Expiration Date: | February 2024 | | |
| Lot Number: | 2606/0423 | | |
| Country of Origin: | South Africa | | |
| Harvesting protocol: | In accordance with the conservation control authority of South Africa | | |
| PARAMETER | SPECIFICATION | METHOD | RESULT |
| Plant Part Used: | Whole Herba | Visual | Whole Herba/Pass |
| Extraction Method: | Ethanolic Extraction | Proprietary | Verified on BMR |
| Plant/Extract Ratio: | 20:1 [DER] | S/L | Verified on BMR |
| Product Characterization: | As per in-house Ref. No. DV-17 | GC-MS | Verified/No. DV-17 |
| Appearance: | Fine powder | Visual | Powder/Pass |
| Colour: | Light Tan to Beige | Visual | Light Tan/Pass |
| Odour: | Characteristic | Organoleptic | Characteristic/Pass |
| Solubility: | 16 g/Lt in H2O @ 20* C. (±10%) | Physical | 16.2 g/Lt/Pass |
| Main Active Ingredient: | 7.6-8.4% Mesembrine | HPLC | 8.03%/Pass |
| Main Active Ingredient: | Mannitol | 3rd Party | Present as per BMR |
| Particle Size: | 60-100 micron | Sieve | Pass/Complies |
| Moisture: | MNT 5% | AOAC 950.46 | 2.2%/Pass |

| | |
|---|---|
| Storage and Handling: | Closed, sealed storage container. |
| | Cool and dry conditions - (20-25* C. @ 65% RH) |
| | This product is non-GMO |

In some embodiments, the kanna extract has the following characteristics, as represented by the exemplary certificate of analysis below.

Typical Certificate of Analysis

| Product: | SCELETIUM TORTUOSUM ET2 PHYTO-EXTRACT | | |
|---|---|---|---|
| Botanical/Plant Material: | Sceletium tortuosum | | |
| Manufactured By: | ULTRAKANNA LLC | | |
| Production Date: | 15 Oct. 2020 | | |
| Expiration Date: | 15 Oct. 2022 | | |
| Lot Number: | 1919/1020 | | |
| Country of Origin: | South Africa | | |
| Harvesting protocol: | In accordance with the conservation control authority of South Africa | | |
| PARAMETER | SPECIFICATION | METHOD | RESULT |
| Plant Part Used: | Whole Herba | Visual | Pass/Verified |
| Appearance: | Fine powder | Visual | Pass/Verified |
| Colour: | Off White to Pale Tan | Visual | Pass/Verified |
| Odour and Taste: | Odour and Taste Characteristic | Organoleptic | Pass/Verified |
| Plant/Extract Ratio: | 11:1 [DER] | S/L | Pass/Verified |
| Main Active Ingredient: | Primary Alkaloid - Mesembrine | HPLC | 2.0%* |
| Excipient: | Mannitol | 3rd Party CoA | Present |
| Solubility: | Soluble in Water | Physical | Pass/Verified |
| Moisture: | MNT 5% | AOAC 950.46 | 3.0% |
| TLC (Thin layer | As per Reference Standard | TLC | Pass/Verified |
| Chromatography:) | | | |
| Particle/Mesh Size: | Between 60-100 micron | Sieve | Complies |

Heavy Metals

| | | | |
|---|---|---|---|
| Lead: | <3.0 mg/kg | AOAC 984.27 | Raw materials complies as |
| Cadmium: | <1.0 mg/kg | AOAC 984.27 | per standards |
| Mercury | <0.10 mg/kg | AOAC 984.27 | |
| Coliforms: | <10 cfu/g | ISO 4832 | Complies |
| Total Aerobic Count: | <1000 cfu/g | MFHPB-18 | Complies |
| Yeast and Mould: | <100 cfu/g | AOAC | Complies |
| | | 6.1: 1997 | |
| Escherchia Coli: | Absent | ISO 16649-2 | Non-detected |
| Staph. Auerus: | Absent | ISO 6888-2 | Non-detected |
| Salmonella: | Absent | SANS | Non detected |
| | | 6579: 2003 | |

| | |
|---|---|
| Storage and Handling: | Closed, sealed storage container. |
| | Cool and dry conditions - at or below 25* C. |
| | This product is non-GMO |
| | *5% Variance permitted |

In some embodiments, wherein a disclosed empathogenic botanical formulation comprises a kanna extract, the kanna extract has an average particle size, or range of particle sizes (i.e., particle size distribution) that can be controlled by manipulating the grinding conditions, and/or by physically separating and selecting for particles of a particular size, e.g., by using mesh sieves. In some embodiments, the particle size of the kanna extract is less than about 200 microns, less than about 150 microns, less than about 125 microns, less than about 100 microns, less than about 75 microns, less than about 50 microns, or less than about 25 microns. In some embodiments, the particle size of the kanna extract is less than about 75 microns. In some embodiments, the particle size of the kanna extract is between about 10 microns and 200 microns. In some embodiments, the particle size of the kanna extract is between about 10 microns and 150 microns. In some embodiments, the particle size of the kanna extract is between about 10 microns and 100 microns. In some embodiments, the particle size of the kanna extract is between about 50 microns and 200 microns. In some embodiments, the particle size of the kanna extract is between about 50 microns and 150 microns. In some embodiments, the particle size of the kanna extract is between about 50 microns and 100 microns. In some embodiments, the particle size of the kanna extract is between about 60 microns and 200 microns. In some embodiments, the particle size of the kanna extract is between about 60 microns and 150 microns. In some embodiments, the particle size of the kanna extract is between about 60 microns and 100 microns.

In some embodiments, wherein a disclosed empathogenic botanical formulation comprises a kanna extract, the formulation comprises more than one kanna extract. In embodiments, the formulation comprises a first kanna extract and a second kanna extract. In embodiments, the combination of kanna extracts in a disclosed formulation creates a synergistic chemical profile of kanna components (e.g., kanna alkaloids such as mesembrine) and/or physical properties that contribute to the processability, ease of manufacture, and overall effects (e.g., synergistic effects, empathogenic effects) of the formulation. In embodiments, a disclosed formulation comprises a first kanna extract with a high mesembrine content (e.g., about 7-9% mesembrine) to provide the user with an initial observable effect; and one or more additional kanna components (e.g., a second, lower-mesembrine kanna extract having a mesembrine content of about 2-3%, and/or kanna plant material) to provide continuous release of full-spectrum kanna alkaloids over an extended period of time. For example, in some such embodiments, the formulation comprises high-mesembrine kanna extract, lower-mesembrine kanna extract, and full-spectrum kanna plant powder in a ratio of 2:1:1.

In some embodiments wherein the formulation comprises a first kanna extract and a second kanna extract, the weight ratio of the first kanna extract to the second kanna extract is between about 1:10 and 10:1. In embodiments, the weight ratio of the first kanna extract to the second kanna extract is between about 1:10 and 1:1. In embodiments, the weight ratio of the first kanna extract to the second kanna extract is between about 1:1 and 10:1. In embodiments, the weight ratio of the first kanna extract to the second kanna extract is between about 1:1 and 5:1. In embodiments, the weight ratio of the first kanna extract to the second kanna extract is between about 1:1 and 3:1. In embodiments, the weight ratio of the first kanna extract to the second kanna extract is about 1:1, 2:1, or 3:1, including ranges in between these ratios.

In embodiments wherein the formulation comprises kanna plant material (e.g., fermented kanna plant powder, unfermented kanna plant powder, or a mixture) and one or more kanna extracts, the weight ratio of the kanna plant material and the total weight of the kanna extract(s) is between about 1:1 and 1:10. In embodiments, the weight ratio of the kanna plant material and the total weight of the kanna extract(s) is between about 1:1 and 1:5. In some embodiments, the weight ratio of the kanna plant material and the total weight of the kanna extract(s) is about 1:1, 1:2, 1:3, 1:4, or 1:5, including ranges in between these ratios.

In some embodiments, wherein a disclosed empathogenic botanical formulation comprises a kanna extract, the kanna extract is present at an amount of between about 1% and 10% (v/v) of the total plant material in the formulation (i.e., the sum of the plant material in the kanna composition and the synergistic plant composition). In some embodiments, the kanna extract is present at an amount of about 1%, 2%, 3%, 4%, 5%, 6%, 7%, 8%, 9%, or 10%, including ranges in between these values. In some embodiments, the kanna extract is present at an amount of about 4%.

b. Synergistic Plant Compositions

In some embodiments, disclosed empathogenic botanical formulations comprise an additional plant composition that provides synergistic effects (herein, a “synergistic plant composition”).

In some embodiments, a disclosed synergistic plant composition comprises Acacia senegal. Acacia senegal (also known as Senegalia senegal and many common names, including gum acacia, gum arabic tree, Sudan gum, Kher, or Khor) is a small, spiny, deciduous tree native to semi-desert regions of Sub-Saharan Africa and South Asia. Acacia senegal can produce a natural gum (“gum arabic” or “gum acacia”) that consists of a complex mixture of glycoproteins and polysaccharides (primarily arabinose and galactose). Gum arabic is used in the food industry and soft-drink industry as a stabilizer, and in herbal medicine, the gum is sometimes used as a binding agent, to stabilize emulsions, and to produce a matrix for topical formulations. Consuming gum arabic has also been shown to impart direct benefits, such as reducing post-prandial glucose (PPG) and post-prandial insulin (PPI) levels in both normal-weight and overweight subjects (Bongartz, et al. Food Sci. Nutr. 2022; 13(4):424-438).

In some embodiments, a disclosed synergistic plant composition comprises Acacia senegal plant material. In some embodiments, the Acacia senegal plant material is gum arabic. In some embodiments, the Acacia senegal plant material is Acacia senegal plant powder. Acacia senegal plant powder is commercially available, or can be produced by harvesting, drying, and grinding (i.e., milling, pulverizing, or crushing) the plant mass into a fine powder. In some embodiments, the Acacia senegal plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Acacia senegal plant powder is between about 0.1 and 1.0 mm. In some embodiments, the particle size is about 0.1 mm. In some embodiments, the particle size is about 0.2 mm. In some embodiments, the particle size is about 0.3 mm. In some embodiments, the particle size is about 0.4 mm. In some embodiments, the particle size is about 0.5 mm. In some embodiments, the particle size is about 0.6 mm. In some embodiments, the particle size is about 0.7 mm. In some embodiments, the particle size is about 0.8 mm. In some embodiments, the particle size is about 0.9 mm. In some embodiments, the particle size is about 1.0 mm. In some embodiments, the particle size is less than 0.1 mm (e.g., less than 100, 90, 80, 70, 60, 50, 40, 30, 20, or 10 μm). In some embodiments, the particle size is greater than 1.0 mm (e.g., greater than 1.0, 1.1, 1.2, 1.3, 1.4, or 1.5 mm). In some embodiments, the particle size is between about 0.1 mm and 1.0 mm, 0.2 mm and 0.9 mm, 0.3 mm and 0.7 mm, or 0.4 mm and 0.6 mm. In certain preferred embodiments, the particle size of the Acacia senegal plant powder is about 0.5 mm.

In some embodiments, a disclosed synergistic plant composition comprises between about 40% and 80% (v/v) of Acacia senegal plant material. In some embodiments, the disclosed synergistic plant composition comprises between about 50% and 70% (v/v) of Acacia senegal plant material. In embodiments, the disclosed synergistic plant composition comprises between about 55% and 65% (v/v) of Acacia senegal plant material. In embodiments, the disclosed synergistic plant composition comprises between about 60% and 65% (v/v) of Acacia senegal plant material. In embodiments, the disclosed synergistic plant composition comprises about 60%, 61%, 62%, 63%, 64%, or 65% (v/v) of Acacia senegal plant material.

In embodiments, a disclosed synergistic plant composition comprises Acmella oleracea. Acmella oleracea (also known as Spilanthes acmella, and various common names including paracress and toothache plant) is a well-known antitoothache plant that has been used as traditional medicine for many purposes (Sahu, et al. Pharmacologyonline 2011; 2:1105-1110; Prachayasittikul, et al., EXCLI J. 2013; 12:291-312).

In some embodiments, a disclosed synergistic plant composition comprises Acmella oleracea plant material. In some embodiments, the Acmella oleracea plant material is Acmella oleracea plant powder. Acmella oleracea plant powder is commercially available, or can be produced by harvesting, drying, and grinding (i.e., milling, pulverizing, or crushing) the plant mass into a fine powder. In some embodiments, the Acmella oleracea plant powder is produced using a high-speed grinder. In some embodiments, the Acmella oleracea plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Acmella oleracea plant powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In embodiments, the particle size is between about 90 and 110 μm. In some preferred embodiments, the particle size is about 100 μm.

In some embodiments, a disclosed synergistic plant composition comprises between about 5% and 40% (v/v) of Acmella oleracea plant material. In some embodiments, a disclosed synergistic plant composition comprises between about 10% and 30% (v/v) of Acmella oleracea plant material. In some embodiments, a disclosed synergistic plant composition comprises between about 15% and 25% (v/v) of Acmella oleracea plant material. In some embodiments, a disclosed synergistic plant composition comprises between about 17.5 and 22.5% (v/v) of Acmella oleracea plant material. In some embodiments, a disclosed synergistic plant composition comprises about 15%, 16%, 17%, 18%, 19%, 20%, 21%, 22%, 23%, 24%, or 25% (v/v) of Acmella oleracea plant material.

In some embodiments, a disclosed synergistic plant composition comprises Saussurea involucrata. Saussurea involucrata is a rare species of flowering plant in the Asteraceae family that grows in the high mountains of central Asia. Also known as “snow lotus,” Saussurea involucrata is widely used in traditional medicine and has been shown to have anti-neoplastic, anti-inflammatory, analgesic, anti-oxidative, anti-fatigue, anti-aging, anti-hypoxic, neuroprotective and immunomodulating effects (Chik, et al., J. Ethnopharmacol. 2015; 172(22):44-60).

In some embodiments, a disclosed synergistic plant composition comprises Saussurea involucrata plant material. In some embodiments, the Saussurea involucrata plant material is Saussurea involucrata plant powder. Saussurea involucrata plant powder is commercially available, or can be produced by harvesting, drying, and grinding (i.e., milling, pulverizing, or crushing) the plant mass into a fine powder. In some embodiments, the Saussurea involucrata plant powder is produced using a high-speed grinder. In embodiments, the Saussurea involucrata plant powder has an average particle size, or range of particle sizes (i.e., particle size distribution) that can be controlled by manipulating the grinding conditions, and/or by physically separating and selecting for particles of a particular size, for example by using mesh sieves. In embodiments, a disclosed synergistic plant composition comprises Saussurea involucrata microglobules.

In some embodiments, the particle size of the Saussurea involucrata plant powder (i.e., the Saussurea involucrata microglobules) is between about 0.1 and 2.0 mm. In some embodiments, the particle size is about 0.1 mm. In some embodiments, the particle size is about 0.2 mm. In some embodiments, the particle size is about 0.3 mm. In some embodiments, the particle size is about 0.4 mm. In some embodiments, the particle size is about 0.5 mm. In some embodiments, the particle size is about 0.6 mm. In some embodiments, the particle size is about 0.7 mm. In some embodiments, the particle size is about 0.8 mm. In some embodiments, the particle size is about 0.9 mm. In some embodiments, the particle size is about 1.0 mm. In some embodiments, the particle size is about 1.1 mm. In some embodiments, the particle size is about 1.2 mm. In some embodiments, the particle size is about 1.3 mm. In some embodiments, the particle size is about 1.4 mm. In some embodiments, the particle size is about 1.5 mm. In some embodiments, the particle size is about 1.6 mm. In some embodiments, the particle size is about 1.7 mm. In some embodiments, the particle size is about 1.8 mm. In some embodiments, the particle size is about 1.9 mm. In some embodiments, the particle size is about 2.0 mm. In some embodiments, the particle size is less than 0.1 mm (e.g., less than 100, 90, 80, 70, 60, 50, 40, 30, 20, or 10 μm). In some embodiments, the particle size is greater than 2.0 mm (e.g., greater than 2.0, 2.1, 2.2, 2.3, 2.4, or 2.5 mm). In some embodiments, the particle size is between about 0.1 mm and 2.0 mm, 0.5 mm and 1.5 mm, 0.75 mm and 1.25 mm, or 0.9 mm and 1.1 mm. In certain preferred embodiments, the particle size of the Saussurea involucrata plant powder is about 1.0 mm.

In some embodiments, a disclosed synergistic plant composition comprises between about 0.1% and 5% (v/v) of Saussurea involucrata powder extract. In some embodiments, a disclosed synergistic plant composition comprises between about 0.5% and 5% (v/v) of Saussurea involucrata powder extract. In some embodiments, a disclosed synergistic plant composition comprises between about 1% and 3% (v/v) of Saussurea involucrata powder extract. In some embodiments, a disclosed synergistic plant composition comprises about 1%, 1.25%, 1.5%, 1.75%, 2%, 2.5%, or 3% (v/v) of Saussurea involucrata powder extract.

In some embodiments, a disclosed synergistic plant composition comprises a species of the Lavandula genus. In preferred embodiments, a disclosed synergistic plant composition comprises Lavandula angustifolia. Lavandula angustifolia, also known as English lavender, is one of 47 known species of flowering plants in the Lavandula genus. Lavandula angustifolia is the most common Lavandula species, but others (e.g., L. stoechas, L. dentata, L. multifida) are also commonly grown as ornamental plants. Although, in preferred embodiments, a disclosed synergistic plant composition comprises Lavandula angustifolia, another species of Lavandula may be substituted for Lavandula angustifolia in other embodiments disclosed herein. For example, in some embodiments, a disclosed synergistic plant composition comprises Lavandula stoechas. In other embodiments, disclosed synergistic plant composition comprises Lavandula dentata. In other embodiments, disclosed synergistic plant composition comprises Lavandula multifida.

Lavandula plant material and extracts produced therefrom have been shown to have anxiolytic, mood stabilizer, sedative, analgesic, and anticonvulsive and neuroprotective properties (Koulivand, et al. Evid Based Complement Alternat Med. 2013, 681304).

In some embodiments, a disclosed synergistic plant composition comprises Lavandula angustifolia plant material. In some embodiments, the Lavandula angustifolia plant material is Lavandula angustifolia plant powder. Lavandula angustifolia plant powder is commercially available, or can be produced by harvesting, drying, and grinding (i.e., milling, pulverizing, or crushing) the plant mass into a fine powder. In some embodiments, the Lavandula angustifolia plant powder is produced using a high-speed grinder. In embodiments, the Lavandula angustifolia plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Lavandula angustifolia plant powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In embodiments, the particle size is between about 90 and 110 μm. In some preferred embodiments, the particle size is about 100 μm.

In some embodiments, a disclosed synergistic plant composition comprises between about 0.1% and 5% (v/v) of Lavandula angustifolia plant material. In some embodiments, a disclosed synergistic plant composition comprises between about 0.5% and 5% (v/v) of Lavandula angustifolia plant material. In some embodiments, a disclosed synergistic plant composition comprises between about 1% and 3% (v/v) of Lavandula angustifolia plant material. In some embodiments, a disclosed synergistic plant composition comprises about 1%, 1.5%, 2% 2.5%, or 3% (v/v) of Lavandula angustifolia plant material.

In some embodiments, a disclosed synergistic plant composition comprises a species of the Mentha genus, such as Mentha spicata (i.e., spearmint) or Mentha piperita (i.e., peppermint). In some embodiments, a disclosed synergistic plant composition comprises Mentha spicata. Mentha sp. plant material and extracts have many uses in traditional medicine, and have been shown to exert central nervous system (CNS) effects, such as antioxidant and MAO-A inhibitory activity, and GABAA affinity (López, et al. Phytother Res. 2010; 24(6):869-874).

In some embodiments, a disclosed synergistic plant composition comprises Mentha spicata plant material. In some embodiments, a disclosed synergistic plant composition comprises Mentha spicata extract. In some embodiments, the extract is a liquid extract. In some embodiments, the extract is a solid (powder) extract. In some embodiments, the extract is Mentha spicata white powder extract.

In some embodiments, the particle size of the Mentha spicata white powder extract is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm.

In embodiments, a disclosed synergistic plant composition comprises between about 0.1% and 5% (v/v) of Mentha spicata white powder extract. In embodiments, a disclosed synergistic plant composition comprises between about 0.5% and 5% (v/v) of Mentha spicata white powder extract. In embodiments, a disclosed synergistic plant composition comprises between about 1% and 3% (v/v) of Mentha spicata white powder extract. In embodiments, a disclosed synergistic plant composition comprises about 1%, 1.125%, 1.25%, 1.5%, 1.75%, 1.875%, 2%, 2.5%, or 3% (v/v) of Mentha spicata white powder extract.

In embodiments, a disclosed synergistic plant composition comprises ginger (Zingiber officinale). Z. officinale is a flowering plant whose rhizome is widely used as a culinary spice and a traditional medicine (Shahrajabian, et al. Acta Agric Scand B Soil Plant Sci. 2019; 69:546-556). Z. officinale has been used as an herbal medicine to treat various symptoms including nausea, vomiting, pain, and cold symptoms; and it has been shown to have anti-inflammatory, anti-apoptotic, anti-tumour activities, anti-pyretic, anti-platelet, anti-tumourigenic, anti-hyperglycaemic, antioxidant anti-diabetic, anti-clotting, and analgesic properties (id.).

In some embodiments, the Zingiber officinale plant material is Zingiber officinale root powder. Zingiber officinale root powder is commercially available, or can be produced by harvesting, drying, and grinding (i.e., milling, pulverizing, or crushing) the rhizome mass into a fine powder. In some embodiments, the Zingiber officinale root powder is produced using a high-speed grinder. In some embodiments, the Zingiber officinale root powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Zingiber officinale root powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm. In some embodiments, the particle size is about 90 μm.

In some embodiments, a disclosed synergistic plant composition comprises between about 0.1% and 5% (v/v) of Zingiber officinale plant material (e.g., root powder). In some embodiments, a disclosed synergistic plant composition comprises between about 0.5% and 5% (v/v) of Zingiber officinale plant material (e.g., root powder). In some embodiments, a disclosed synergistic plant composition comprises between about 1% and 5% (v/v) of Zingiber officinale plant material (e.g., root powder). In some embodiments, a disclosed synergistic plant composition comprises about 0.5%, 1%, 1.5%, 2%, 2.5%, 3%, 3.5%, 4%, 4.5%, or 5% of Zingiber officinale plant material (e.g., root powder).

In some embodiments, a disclosed synergistic plant composition comprises chamomile plant material. Chamomile is the common name for several plant species in the Asteraceae family, including Matricaria chamomilla (often called “German chamomile”) and Chamaemelum nobile (Roman, English, or garden chamomile). The dried flowers of the chamomile plant may have medicinal properties, and chamomile preparations are commonly used in herbal medicine to alleviate many ailments, including hay fever, inflammation, muscle spasms, menstrual disorders, insomnia, ulcers, wounds, gastrointestinal disorders, rheumatic pain and hemorrhoids (Srivastava, et al. Mol. Med. Rep. 2010; 3(6):895-901).

In some embodiments, the chamomile plant material is Chamaemelum nobile plant powder. Chamaemelum nobile plant powder is commercially available, or can be produced by harvesting, drying, and grinding (i.e., milling, pulverizing, or crushing) the plant mass (e.g., dried flowers) into a fine powder. In some embodiments, the Chamaemelum nobile plant powder is produced using a high-speed grinder. In some embodiments, the Chamaemelum nobile plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Chamaemelum nobile plant powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm.

In some embodiments, a disclosed synergistic plant composition comprises between about 0.1% and 5% (v/v) of Chamaemelum nobile plant material (e.g., plant powder). In some embodiments, a disclosed synergistic plant composition comprises between about 0.5% and 5% (v/v) of Chamaemelum nobile plant material (e.g., plant powder). In some embodiments, a disclosed synergistic plant composition comprises between about 1% and 5% (v/v) of Chamaemelum nobile plant material (e.g., plant powder). In some embodiments, a disclosed synergistic plant composition comprises about 0.5%, 1%, 1.5%, 2%, 2.5%, 3%, 3.5%, 4%, 4.5%, or 5% of Chamaemelum nobile plant material (e.g., plant powder).

In some embodiments, a disclosed synergistic plant composition comprises Galium odoratum (also known as Asperula odorata or “sweet woodruff”). Sweet woodruff and extracts produced therefrom have a history of traditional use in Ukraine, and have been investigated for its antihypoxic and sedative effects (Sergeevna, et al. Pharmacogn. Commn. 2015; 5(4):233-236). In some embodiments, a disclosed synergistic plant composition comprises sweet woodruff plant material.

In some embodiments, wherein a synergistic plant composition comprises a sweet woodruff extract, the extract may be a solid (powder) extract or a liquid (aqueous or alcoholic) extract. In embodiments wherein a synergistic plant composition comprises a solid (powder) sweet woodruff extract, the powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the extract is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm.

In some embodiments, a disclosed synergistic plant composition comprises between about 0.1% and 5% (v/v) of sweet woodruff (e.g., sweet woodruff extract). In some embodiments, a disclosed synergistic plant composition comprises between about 0.5% and 5% (v/v) of sweet woodruff (e.g., sweet woodruff extract). In some embodiments, a disclosed synergistic plant composition comprises between about 1% and 5% (v/v) of sweet woodruff (e.g., sweet woodruff extract). In some embodiments, a disclosed synergistic plant composition comprises about 0.5%, 1%, 1.5%, 2%, 2.5%, 3%, 3.5%, 4%, 4.5%, or 5% of sweet woodruff (e.g., sweet woodruff extract).

In some embodiments, a disclosed synergistic plant composition comprises Silybum marianum. Silybum marianum is also known by several common names, including milk thistle. Amongo the potentially therapeutic compounds, Silybum marianum contains silymarin, a mixture of three flavonolignans (silybin, silydianin and silycristin) (Karkanis, et al. Ind Crops Prod. 2011; 34(1):825-830). The seeds and the whole plant are used in herbal medicine (id.).

In some embodiments, the Silybum marianum plant material is Silybum marianum plant powder or seed powder. Silybum marianum plant material is commercially available, or can be produced by harvesting, drying, and grinding (i.e., milling, pulverizing, or crushing) the plant mass or seed mass into a fine powder. In some embodiments, the Silybum marianum plant material has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Silybum marianum plant material is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm.

In some embodiments, a disclosed synergistic plant composition comprises between about 0.1% and 5% (v/v) of Silybum marianum plant material (e.g., plant powder or seed powder). In some embodiments, a disclosed synergistic plant composition comprises between about 0.5% and 5% (v/v) of Silybum marianum plant material (e.g., plant powder or seed powder). In some embodiments, a disclosed synergistic plant composition comprises between about 1% and 5% (v/v) of Silybum marianum plant material (e.g., plant powder). In some embodiments, a disclosed synergistic plant composition comprises about 0.5%, 1%, 1.5%, 2%, 2.5%, 3%, 3.5%, 4%, 4.5%, or 5% of Silybum marianum plant material (e.g., plant powder).

In some embodiments, a disclosed synergistic plant composition comprises rooibos (Aspalathus linearis). Aspalathus linearis is a plant of the Fabaceae family that growns natively in South Africa. A. linearis has a long history of traditional use in South Africa (Joubert, et al. J. Ethnopharmacol. 2008; 119(3):376-412).

In some embodiments, the Aspalathus linearis plant material is Aspalathus linearis plant powder. Aspalathus linearis plant powder is commercially available, or can be produced by harvesting, drying, and grinding (i.e., milling, pulverizing, or crushing) the rhizome mass into a fine powder. In some embodiments, the Aspalathus linearis plant powder is produced using a high-speed grinder. In some embodiments, the Aspalathus linearis plant powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the Aspalathus linearis plant powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm. In some embodiments, the particle size is between about 90 and 110 μm. In some embodiments, the particle size is about 100 μm.

In some embodiments, a disclosed synergistic plant composition comprises between about 0.1% and 5% (v/v) of Aspalathus linearis plant material (e.g., plant powder). In some embodiments, a disclosed synergistic plant composition comprises between about 0.5% and 5% (v/v) of Aspalathus linearis plant material (e.g., plant powder). In some embodiments, a disclosed synergistic plant composition comprises between about 1% and 5% (v/v) of Aspalathus linearis plant material (e.g., plant powder). In some embodiments, a disclosed synergistic plant composition comprises about 0.5%, 1%, 1.5%, 2%, 2.5%, 3%, 3.5%, 4%, 4.5%, or 5% of Aspalathus linearis plant material (e.g., plant powder).

In some embodiments, a disclosed synergistic plant composition comprises rose (Rosa sp.) plant material. Rosa sp. is a genus of flowering plants that includes many species known for their beautiful and fragrant flowers, commonly referred to as roses. Roses have been used in various cultural contexts for ornamental, culinary, and medicinal purposes. In traditional medicine, roses have been used to treat a range of conditions, including skin irritation, sore throats, and digestive issues (Mileva et al. Biomolecules 2021; 11(1):127). Additionally, rose extracts have been studied for their anti-inflammatory, antioxidant, and antimicrobial properties (id.).

In some embodiments, the rose plant material is rose petal powder. Rose petal powder is commercially available, or can be produced by harvesting, drying, and grinding (i.e., milling, pulverizing, or crushing) rose petal mass into a fine powder. In embodiments, the rose petal powder is produced using a high-speed grinder. In embodiments, the rose petal powder has an average particle size, or range of particle sizes (i.e., particle size distribution) that can be controlled by manipulating the grinding conditions, and/or by physically separating and selecting for particles of a particular size, for example by using mesh sieves.

In some embodiments, the particle size of the rose petal powder is between about 10 and 1000 μm. In some embodiments, the particle size is about 10 μm. In some embodiments, the particle size is about 20 μm. In some embodiments, the particle size is about 50 μm. In some embodiments, the particle size is about 75 μm. In some embodiments, the particle size is about 100 μm. In some embodiments, the particle size is about 125 μm. In some embodiments, the particle size is about 150 μm. In some embodiments, the particle size is about 200 μm. In some embodiments, the particle size is about 250 μm. In some embodiments, the particle size is about 500 μm. In some embodiments, the particle size is about 1000 μm. In some embodiments, the particle size is between about 10 and 1000 μm. In some embodiments, the particle size is between about 50 and 500 μm. In some embodiments, the particle size is between about 50 and 200 μm. In some embodiments, the particle size is between about 50 and 150 μm. In some embodiments, the particle size is between about 75 and 125 μm.

In some embodiments, a disclosed synergistic plant composition comprises between about 0.1% and 5% (v/v) of rose plant material (e.g., rose petal powder). In some embodiments, a disclosed synergistic plant composition comprises between about 0.5% and 5% (v/v) of rose plant material (e.g., rose petal powder). In some embodiments, a disclosed synergistic plant composition comprises between about 1% and 5% (v/v) of rose plant material (e.g., rose petal powder). In some embodiments, a disclosed synergistic plant composition comprises about 0.5%, 1%, 1.5%, 2%, 2.5%, 3%, 3.5%, 4%, 4.5%, or 5% of rose plant material (e.g., rose petal powder).

In some embodiments, the synergistic plant composition comprises a combination of any two or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any three or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any four or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any five or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any six or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any seven or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of any eight or more of: Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material. In some embodiments, the synergistic plant composition comprises a combination of Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, Rosa sp. plant material, and Aspalathus linearis plant material.

In some embodiments, the synergistic plant composition comprises Acacia senegal plant material and Acmella oleracea plant material. In some embodiments, the synergistic plant composition comprises Acacia senegal plant material and Saussurea involucrata plant material. In some embodiments, the synergistic plant composition comprises Acacia senegal plant material and Lavandula angustifolia plant material. In some embodiments, the synergistic plant composition comprises Acacia senegal plant material and Mentha spicata extract. In some embodiments, the synergistic plant composition comprises Acacia senegal plant material, Acmella oleracea plant material, and Saussurea involucrata plant material. In some embodiments, the synergistic plant composition comprises Acacia senegal plant material, Acmella oleracea plant material, and Lavandula angustifolia plant material. In some embodiments, the synergistic plant composition comprises Acacia senegal plant material, Acmella oleracea plant material, and Mentha spicata extract. In some embodiments, the synergistic plant composition comprises Acacia senegal plant material and Zingiber officinale plant material. In some embodiments, the synergistic plant composition comprises Acacia senegal plant material and Chamaemelum nobile plant material. In some embodiments, the synergistic plant composition comprises Acacia senegal plant material and sweet woodruff extract. In some embodiments, the synergistic plant composition comprises Acacia senegal plant material and Silybum marianum plant material. In some embodiments, the synergistic plant composition comprises Acacia senegal plant material and Aspalathus linearis plant material.

In some embodiments, the synergistic plant composition comprises Acacia senegal plant material, Acmella oleracea plant material, and Saussurea involucrata plant material. In some embodiments, the synergistic plant composition comprises Acacia senegal plant material, Acmella oleracea plant material, and Lavandula angustifolia plant material. In some embodiments, the synergistic plant composition comprises Acacia senegal plant material, Acmella oleracea plant material, and Mentha spicata extract. In some embodiments, the synergistic plant composition comprises Acacia senegal plant material, Acmella oleracea plant material, and Zingiber officinale plant material. In some embodiments, the synergistic plant composition comprises Acacia senegal plant material, Acmella oleracea plant material, and Chamaemelum nobile plant material. In some embodiments, the synergistic plant composition comprises Acacia senegal plant material, Acmella oleracea plant material, and sweet woodruff extract. In some embodiments, the synergistic plant composition comprises Acacia senegal plant material, Acmella oleracea plant material, and Silybum marianum plant material. In some embodiments, the synergistic plant composition comprises Acacia senegal plant material, Acmella oleracea plant material, and Aspalathus linearis plant material.

In some embodiments, the synergistic plant composition comprises Acacia senegal plant material, Saussurea involucrata plant material, and Lavandula angustifolia plant material. In some embodiments, the synergistic plant composition comprises Acacia senegal plant material, Saussurea involucrata plant material, and Mentha spicata extract. In some embodiments, the synergistic plant composition comprises Acacia senegal plant material, Saussurea involucrata plant material, and Zingiber officinale plant material. In some embodiments, the synergistic plant composition comprises Acacia senegal plant material, Saussurea involucrata plant material, and Chamaemelum nobile plant material. In some embodiments, the synergistic plant composition comprises Acacia senegal plant material, Saussurea involucrata plant material, and sweet woodruff extract. In some embodiments, the synergistic plant composition comprises Acacia senegal plant material, Saussurea involucrata plant material, and Silybum marianum plant material. In some embodiments, the synergistic plant composition comprises Acacia senegal plant material, Saussurea involucrata plant material, and Aspalathus linearis plant material.

In some embodiments, the synergistic plant composition comprises Acacia senegal plant material, Lavandula angustifolia plant material, and Acmella oleracea plant material. In some embodiments, the synergistic plant composition comprises Acacia senegal plant material, Lavandula angustifolia plant material, and Saussurea involucrata plant material. In some embodiments, the synergistic plant composition comprises Acacia senegal plant material, Lavandula angustifolia plant material, and Mentha spicata extract. In some embodiments, the synergistic plant composition comprises Acacia senegal plant material, Lavandula angustifolia plant material, and Zingiber officinale plant material. In some embodiments, the synergistic plant composition comprises Acacia senegal plant material, Lavandula angustifolia plant material, and Chamaemelum nobile plant material. In some embodiments, the synergistic plant composition comprises Acacia senegal plant material, Lavandula angustifolia plant material, and sweet woodruff extract. In some embodiments, the synergistic plant composition comprises Acacia senegal plant material, Lavandula angustifolia plant material, and Silybum marianum plant material. In some embodiments, the synergistic plant composition comprises Acacia senegal plant material, Lavandula angustifolia plant material, and Aspalathus linearis plant material.

In some embodiments, the synergistic plant composition comprises Acacia senegal plant material, Mentha spicata extract, and Acmella oleracea plant material. In some embodiments, the synergistic plant composition comprises Acacia senegal plant material, Mentha spicata extract, and Saussurea involucrata plant material. In some embodiments, the synergistic plant composition comprises Acacia senegal plant material, Mentha spicata extract, and Lavandula angustifolia plant material. In some embodiments, the synergistic plant composition comprises Acacia senegal plant material, Mentha spicata extract, and Zingiber officinale plant material. In some embodiments, the synergistic plant composition comprises Acacia senegal plant material, Mentha spicata extract, and Chamaemelum nobile plant material. In some embodiments, the synergistic plant composition comprises Acacia senegal plant material, Mentha spicata extract, and sweet woodruff extract. In some embodiments, the synergistic plant composition comprises Acacia senegal plant material, Mentha spicata extract, and Silybum marianum plant material. In some embodiments, the synergistic plant composition comprises Acacia senegal plant material, Mentha spicata extract, and Aspalathus linearis plant material.

In some embodiments, the synergistic plant composition comprises Acacia senegal plant material, Zingiber officinale plant material, and Acmella oleracea plant material. In some embodiments, the synergistic plant composition comprises Acacia senegal plant material, Zingiber officinale plant material, and Saussurea involucrata plant material. In some embodiments, the synergistic plant composition comprises Acacia senegal plant material, Zingiber officinale plant material, and Lavandula angustifolia plant material. In some embodiments, the synergistic plant composition comprises Acacia senegal plant material, Zingiber officinale plant material, and Mentha spicata extract. In some embodiments, the synergistic plant composition comprises Acacia senegal plant material, Zingiber officinale plant material, and Chamaemelum nobile plant material. In some embodiments, the synergistic plant composition comprises Acacia senegal plant material, Zingiber officinale plant material, and sweet woodruff extract. In some embodiments, the synergistic plant composition comprises Acacia senegal plant material, Zingiber officinale plant material, and Silybum marianum plant material. In some embodiments, the synergistic plant composition comprises Acacia senegal plant material, Zingiber officinale plant material, and Aspalathus linearis plant material.

In some embodiments, the synergistic plant composition comprises Acacia senegal plant material, Chamaemelum nobile plant material, and Acmella oleracea plant material. In some embodiments, the synergistic plant composition comprises Acacia senegal plant material, Chamaemelum nobile plant material, and Saussurea involucrata plant material. In some embodiments, the synergistic plant composition comprises Acacia senegal plant material, Chamaemelum nobile plant material, and Lavandula angustifolia plant material. In some embodiments, the synergistic plant composition comprises Acacia senegal plant material, Chamaemelum nobile plant material, and Mentha spicata extract. In some embodiments, the synergistic plant composition comprises Acacia senegal plant material, Chamaemelum nobile plant material, and Zingiber officinale plant material. In some embodiments, the synergistic plant composition comprises Acacia senegal plant material, Chamaemelum nobile plant material, and sweet woodruff extract. In some embodiments, the synergistic plant composition comprises Acacia senegal plant material, Chamaemelum nobile plant material, and Silybum marianum plant material. In some embodiments, the synergistic plant composition comprises Acacia senegal plant material, Chamaemelum nobile plant material, and Aspalathus linearis plant material.

c. Binding Compositions

In some embodiments, disclosed empathogenic botanical formulations comprise a binding composition. One of the functions of the binding composition can be to hold the components of the formulation together into a cohesive whole. The binding composition may also influence the subjective experience of the formulation, affecting such perceptual factors as texture, mouthfeel, and consistency.

In some embodiments, the binding composition comprises agar. Agar is commonly used as a solid culture medium in microbiology, and as a culinary ingredient (e.g., as a substitute for gelatin, or a thickener in fruit preserves, ice cream, and other desserts). In some embodiments, the agar is high gelling-rate (i.e., high gel strength) agar.

In some embodiments, the binding composition comprises agar, such that the total amount of agar in the empathogenic botanical formulation is between about 0.1% and 10% on a total weight basis. In some embodiments, the amount of agar is between about 1% and 5% of the empathogenic botanical formulation on a weight basis, including about 1%, 2%, 3%, 4%, or 5%. In some embodiments, the binding composition does not comprise agar. In some embodiments, a disclosed empathogenic botanical formulation does not comprise agar. In some embodiments, a disclosed empathogenic botanical formulation does not comprise gelatin. In some embodiments, a disclosed empathogenic botanical formulation does not comprise starch.

In some embodiments, the binding composition comprises guar gum. In some embodiments, the amount of guar gum is between about 1% and 20%, 1% and 15%, 1% and 10%, or 1% and 5% of the empathogenic botanical formulation on a weight basis. In some embodiments, the amount of guar gum is about 1%, 2%, 3%, 4%, 5%, 6%, 7%, 8%, 9%, or 10%. In some embodiments, the composition comprises guar gum such that the ratio of guar gum to Acmella plant material is between about 1:5 and 5:1, including about 1:5, 1:4, 1:3, 1:2, 1:1.9, 1:1.8, 1:1.7, 1:1.6, 1:1.5, 1:1.4, 1:1.3, 1:1.2, 1:1.1, 1:1, 1.1:1, 1.2:1, 1.3:1, 1.4:1, 1.5:1, 1.6:1, 1.7:1, 1.8:1, 1.9:1, 2:1, 3:1, 4:1, 5:1 and ranges in between these ratios. In some embodiments, the particle size of the guar gum is less than about 200 microns, less than about 150 microns, less than about 125 microns, less than about 100 microns, less than about 75 microns, less than about 50 microns, or less than about 25 microns.

In some embodiments, the binding composition comprises vegetable glycerin. In some embodiments, the binding composition comprises vegetable glycerin in an amount such that the weight ratio of vegetable glycerin to agar is between about 1:1 and 2:1, including about 1:1, 1.1:1, 1.2:1, 1.3:1, 1.4:1, 1.5:1, 1.6:1, 1.7:1, 1.8:1, 1.9:1, and 2:1. In some embodiments, the weight ratio of vegetable glycerin to agar is between about 1.6:1 and 1.7:1. In some embodiments, the binding composition comprises vegetable glycerin in an amount such that the weight ratio of vegetable glycerin to guar gum is between about 1:1 and 2:1, including about 1:1, 1.1:1, 1.2:1, 1.3:1, 1.4:1, 1.5:1, 1.6:1, 1.7:1, 1.8:1, 1.9:1, and 2:1. In some embodiments, the weight ratio of vegetable glycerin to guar gum is between about 1.6:1 and 1.7:1.

In some embodiments, the binding composition comprises distilled water. In some embodiments, the binding composition comprises distilled water in an amount such that the weight ratio of water to agar is between about 5:1 and 50:1, including about 5:1, 6:1, 7:1, 8:1, 9:1, 10:1, 11:1, 12:1, 13:1, 14:1, 15:1, 20:1, 25:1, and 50:1. In some embodiments, the weight ratio of water to agar is about 10:1. In some embodiments, the binding composition comprises distilled water in an amount such that the weight ratio of water to guar gum is between about 5:1 and 50:1, including about 5:1, 6:1, 7:1, 8:1, 9:1, 10:1, 11:1, 12:1, 13:1, 14:1, 15:1, 20:1, 25:1, and 50:1. In some embodiments, the weight ratio of water to guar gum is about 10:1.

In some embodiments, the binding composition comprises both vegetable glycerin and distilled water. In some embodiments, the binding composition comprises water and vegetable glycerin in a water:glycerin ratio of between about 1:1 and 20:1 (on a w/w or v/v basis). In some embodiments, the water:glycerin ratio is between about 1:1 and 10:1. In some embodiments, the water:glycerin ratio is between about 10:1 and 20:1. In some embodiments, the water:glycerin ratio is between about 2:1 and 8:1. In some embodiments, the water:glycerin ratio is between about 4:1 and 7:1. In some embodiments, the water:glycerin ratio is between about 6:1 and 7:1. In some embodiments, the water:glycerin ratio is about 1:1, 2:1, 3:1, 4:1, 5:1, 6:1, 7:1, 8:1, 9:1, or 10:1, including ranges in between these ratios.

In some embodiments, the binding composition comprises agar in combination with vegetable glycerin and/or distilled water. In some embodiments, the binding composition comprises agar and vegetable glycerin. In some embodiments, the binding composition comprises agar, vegetable glycerin, and distilled water. In some embodiments, the binding composition comprises agar, vegetable glycerin, and distilled water in a weight ratio of about 1:1.7:10.

In some embodiments, the binding composition comprises guar gum in combination with vegetable glycerin and/or distilled water. In some embodiments, the binding composition comprises guar gum and vegetable glycerin. In some embodiments, the binding composition comprises agar, vegetable glycerin, and distilled water. In some embodiments, the binding composition comprises guar gum, vegetable glycerin, and distilled water in a weight ratio of about 1:1.7:10.

d. Additional Contents and Features of Disclosed Formulations

In some embodiments, disclosed empathogenic botanical formulations comprise an additional excipient. In some embodiments, suitable excipients to be incorporated in disclosed formulations may include a flavoring agent, a sweetener, a colorant, a dye, a pigment, an antioxidant, a binder, a solvent, a humectant, a viscosity modifier, a solubilizer, a complexing agent, a preservative, a pH adjusting agent, an opacifier, a surfactant, a gelling agent, an effervescence material, and combinations thereof. In certain embodiments, the excipients that are included in disclosed formulations are natural so as to minimize irritation and/or allergic reactions to the consumer. In embodiments, one or more excipients in a disclosed formulation is non-naturally occurring.

In some embodiments, disclosed formulations may include a flavoring agent or a mixture of flavoring agents including natural, non-natural, and synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils, essences, limonene, menthone, carvone, menthol, anethole, eucalyptol, anethole, eugenol, cassia, oxanone, a-irisone, propenyl guaiethol, thymol, linalool, benzaldehyde, cineole, cinnamaldehyde, cinnamaldehyde glycerol acetal (CGA), methone glycerol acetal (MGA), 3-I-menthoxypropane-1,2-diol, N-ethyl-p-menthan-3-carboxamine, N,2,3-trimethyl-2-isopropylbutanamide, and combinations thereof. Flavoring agent(s) may be present, individually or in total (if more than one flavoring agent is included), in disclosed formulations in an amount ranging from about 0.1 wt % to about 10 wt % (calculated as the total weight of flavoring agent(s) in the formulation divided by the total weight of the formulation). In some embodiments, a disclosed formulation does not comprise a flavoring agent.

In some embodiments a formulation comprises a flavoring agent, which may be a flavor. A flavor may be any of a liquid flavor, a powder flavor, a powdered encapsulated flavor, a spray-dried flavor, an emulsion flavor, a plant, fruit, vegetable, or other extraction-based flavor, a seasoning, an organic flavor, a natural flavor, a non-GMP flavor, a clean label flavor, a concentrated flavor, a savory flavor, a sweet flavor, a masking flavor, and the like, including combinations thereof. Flavors may be produced using methods known to those in the art, or obtained from flavor suppliers as known in the art, for example from Gold Coast Ingredients, Inc. (GCI) (Commerce, California).

Example flavors include acai, acai blueberry, acidophilous lactic, affogato, agua fresca, al pastor, allspice, almond extract, almond amaretto, almond butter, almond butter vanilla, almond cream, almond fig, almond honey, almond milk, almond oil, almond praline, almond raw, almond roasted, almond toasted, almond vanilla, aloe vera, amaretto, amaretto cappuccino, amaretto sour, amaretto tequila lime, ancho chili, anchovy, anise, annatto, apple baked, apple banana, apple berry, apple candy, apple chai, apple cherry, apple cider, apple cider vinegar, apple cinnamon, apple cinnamon spice, apple essence, apple granny smith, apple green, apple juice, apple pie, apple pippin, apple raspberry, apple red, apple sour, apple spice, apple strawberry, applewood smoke, apricot, apricot brandy, apricot habanero, apricot plum wine, arak rum, artichoke, asian blend, asparagus, asparagus garlic, avocado, bacon, bacon hickory smoke, bacon maple, bacon spicy, bacon ranch, baked crust, baklava, baklava pistachio, balsamic, balsamic strawberry, banana extract, banana fresh, banana berry, banana blueberry, banana bouquet, banana cream, banana cream pie, banana custard, banana honey, banana kiwi, banana macadamia, banana nut, banana pancake, banana peach, banana ripe, banana strawberry, banana walnut bread, bananas foster, barbecue, barbecue grill, barbecue honey, barbecue mango, barbecue spicy, basil, basil thai, bavarian cream, bavarian cream pie, bavarian danish, bay leaf, bay rum, bearnaise sauce, beef au jus, beef, beef fat, beef roasted, beef seasoned, beef steak, beef tallow, beef vegetarian, beer, belgian chocolate, bell pepper, bell pepper onion, bell pepper roasted, bergamot, bergamot orange, berry, berry citrus, berry crunch, berry marionberry, berry mixed, berry punch, berry wildberry, biscuit, bitter almond, bitter, bittersweet chocolate, black bean, black cherry, black currant, black forest, black pepper extract black pepper, black raspberry, black tea, black truffle, black walnut, blackberry, blackberry lemon, blackberry schnapps, bloody mary, bloody mary smoky, bloody mary spicy, blue hawaiian, blue raspberry, blue vanilla, blueberry, blueberry acai, blueberry cheesecake, blueberry clear, blueberry mix, blueberry muffin, blueberry parfait, blueberry pie, blueberry pomegranate, blueberry ripe, bourbon, bourbon maple, boysenberry, boysenberry raspberry, brandy, brandy alexander, brandy apple, brazil nut, bread, bread crust, bread pudding, bread rye, bread sourdough, bread wheat, bread white, broccoli, brown butter, brown sugar, brown sugar cinnamon, brown sugar caramelized, brown sugar maple, bubble gum, buffalo style, buffalo style ranch, burgundy, burnt sugar, butter, butter crème, butter dark, butter derivative, butter lemon orange, butter lemon orange vanilla, butter lemon vanilla, butter light, butter movie theatre, butter pecan, butter rum, butter salty, butter sweet, butter toffee, butter vanilla, butter vanilla citrus, butter vanilla rum, buttercream, buttermilk, butternut, butterscotch, butterscotch chocolate, buttery toffee, cajeta, cajun, cajun hot, cake angel food, cake birthday, cake carrot, cake chocolate, cake coffee, cake devils food, cake donut, cake donut spice, cake lemon, cake red velvet, cake white, cake yellow, calamansi, canadian crème, candy bar, candy cane, candy spiced apple, cantaloupe, cantarine, cappuccino, caramel, caramel apple, caramel custard, caramel french, caramel macchiato, caramel peanut, caramel salted, caramel sauce, caramelized butter, caramelized onion, caraway, cardamom, caribbean bouquet, caribbean jerk, carob, carromatoe, carrot, carrot cake, carrot cake spice, cashew, cashew date, cashew roasted, cassia, cat food, catalina spice, catsup, catsup spice, cauliflower, celery, cereal fruit ring, chablis, chai tea, chai tea latte, chamoy, champagne, champagne pink, char, chardonnay, cheese, cheese five blend, cheese american, cheese blue, cheese cheddar, cheese cream, cheese extra sharp, cheese feta, cheese mozzarella, cheese nacho, cheese old english, cheese parmesan, cheese romano, cheese white cheddar, cheesecake, cheesecake chocolate, cheesecake raspberry, cheesecake strawberry, cherimoya, cherrango, cherries jubilee, cherry, cherry almond, cherry berry, cherry bing, cherry black, cherry blossom, cherry bouquet, cherry burgundy, cherry chateau, cherry candy, cherry cola, cherry dark, cherry dark sweet, cherry fresh, cherry ice, cherry lime, cherry maraschino, cherry mint, cherry morello, cherry n′ berry, cherry pie, cherry pineapple, cherry pit, cherry red, cherry red sour, cherry vanilla, cherry wild, cherry wine, cherry yogurt, cherry yuzu, chestnut, chicken, chicken blackened, chicken broth, chicken dark meat, chicken fat, chicken fried, chicken liver, chicken roasted, chicken vegetarian, chicken and waffles, chicken white meat, chicory root, chilaquiles, chile, chile de arbol, chile verde, chili, chili anaheim, chili garlic, chili green, chili lime, chili mango, chili peach, chili spice, chimichanga, chimichurri, chinese five spice, chinese food, chipotle, chipotle honey, chipotle lime, chipotle smoked, chives sour cream, chocolate extract chocolate, chocolate almond, chocolate anise, chocolate belgian, chocolate bitter sweet type, chocolate brownie, chocolate cake, chocolate candy bar, chocolate cappuccino, chocolate caramel, chocolate cherry, chocolate cherry chip, chocolate chip, chocolate clear, chocolate cookie dark, chocolate cookie dough, chocolate creme, chocolate dark, chocolate double-dutch, chocolate fudge, chocolate fudge double, chocolate hazelnut, chocolate liqueur, chocolate malt type, chocolate milk, chocolate mint, chocolate mocha almond, chocolate mocha coffee, chocolate mousse, chocolate orange, chocolate peanut butter, chocolate raspberry, chocolate swiss, chocolate truffle, chocolate white, chorizo, chrysanthemum, churro, cider vinegar toner, cilantro, cinnamon extract cinnamon, cinnamon blend, cinnamon bouquet, cinnamon bun, cinnamon butter, cinnamon butter rum, cinnamon cloves, cinnamon donut, cinnamon fiery, cinnamon hot red, cinnamon pastry, cinnamon raisin, cinnamon roll, cinnamon spice, cinnamon sugar, cinnamon tahitian, cinnamon vanilla, cinnamon whisky, citric acid solution citrus, citrus fruit punch, citrus habanero, citrus island, citrus lemon, citrus lime, clam, clam chowder, cloud emulsion, clove, cocoa extract cocoa, coconut, coconut chili, coconut chocolate, coconut cream, coconut curry, coconut lychee, coconut macadamia, coconut mango, coconut milk, coconut oil, coconut pineapple, coconut water, coconut vanilla, codfish, coffee extract, coffee bridge, coffee cake, coffee chocolate liqueur, coffee cream, coffee hazelnut, coffee liqueur, coffee mexican, coffee mocha mint, coffee nut, coffee vanilla, coffee vanilla nut, coffee vietnamese, cognac, cola, cola berry, cola cherry, cola vanilla, condensed milk, confectioners, cookie dough, cookies'n cream, coriander, corn, corn caramel, corn chip, corn crunch, corn masa, corn sweet, corn toasted, corn white, corned beef, cotton candy, cotton candy blue, cotton candy pink, crab, cranava, cranberry, cranberry apple, cranberry berry, cranberry raspberry, cranberry walnut, cream, cream cake, cream caramel, cream cheese, cream cheesecake, cream praline, cream soda, cream sweet, cream whipped, creamsicle, creamy caramel, creamy lime, creamy raspberry, creamy strawberry, creamy vanilla, creme brulée, creme de cassis, creme de cocoa, creme de menthe, creme of tartar, crumb cake, cucumber, cucumber melon, cumin, currant puree red, curry, curry red, curry spicy, curry yellow, custard, custard vanilla, daiquiri, daiquiri strawberry, dairy, dairy vanilla, danish, dark plum, date, date nut, date raisin, deep fried, devil's food cake, dill, dirty chai, double dutch chocolate, doughnut, dough sweet, doughnut fried, doughnut blend, dragonfruit, dulce de leche, durian, earl grey, earl grey lavender, edamame, egg, egg cream, egg custard, egg vanilla, egg yolk, eggnog, eggplant roasted, elderberry, enchilada, english caramel, english toffee, english toffee caramel, english walnut, enocianina solution espresso, essence of jamaica, ethyl vanillin replacer, eucalyptus, evaporated milk, evergreen mint, fajita, fajita mild, fajita spicy, fat, fennel, fermented bean, fermented bread, fig, fish, fish sauce, floral bouquet, foenugreek seed, framboise, french caramel, french cruller, french fry, french onion, french silk, french toast, french toast cinnamon, french toast maple, french toast strawberry, french vanilla, fried, fruit, fruit cake, fruit intensifier fresh, fruit mixed, fruit punch, fruit punch pineapple, fruit roll, fruit spice, fudge brickle, fudge double dutch, fuzzy mango, fuzzy navel, fuzzy peach, galanga, garden herb, garden vegetable, garleek, garlic, garlic butter, garlic dill, garlic herb, garlic parmesan, garlic roasted, geranium, gimlet, ginger, ginger ale, ginger beer, ginger bread, ginger bread cookie, ginger honey, ginger lemon, ginger peach, ginger peanut, ginger sauteed, ginger spiced, ginger tea, ginseng, goat cheese, goat milk, golden cherry apple, golden delicious apple, gooseberry, graham, graham cracker, graham cracker cinnamon, graham cracker chocolate, graham cracker honey, graham creamy, grand marnier, grape, grape candy, grape concord, grape skin color, grapefruit, grapefruit pink, grapericot, gravy, green apple, green bell pepper, green chili pepper, green cognac oil, green mango, green mint, green onion, green pepper, green tea, green tea ginger, green tea latte, green tea mint, green tea mango, green tea pomegranate, grenadine, grill, guacamole mild, guacamole spicy, guanabana, guava, guava grapefruit, guava grapefruit lime, guava lime, guava mango, guava passion fruit, guava strawberry, guyabano fruit, habanero chili, habanero chili roasted, habanero ranch, ham, ham honey, hamburger, hamburger vegetarian, hatch chili, hawaiian bouquet, hawaiian fruit, hawaiian rum, hawaiian sweet bread, hazelnut, hazelnut chocolate, hazelnut cinnamon, hazelnut liqueur, hazelnut praline, hazelnut roasted, heath bar type, hemp, herb, hibiscus, hibiscus punch, hibiscus strawberry, hickory smoke, hoisin sauce, hollandaise, honey, honey apple, honey apple cinnamon, honey barbecue, honey chipotle, honey graham, honey lavender, honey lemon, honey lemongrass, honey nut, honey tea, honey vanilla, honeycomb, honeycomb chocolate, honeydew melon, honeydew raspberry, honeysuckle, horchata, horchata rum, horehound, horseradish, hot chili pepper, hot dog, hot herb, hot mustard, hot pepper, hot red pepper, hot sauce, hot spicy blend, hot wing, huckleberry, hurricane, iced tea, iced tea lemon, iced tea orange, iced tea passion fruit, iced tea pomegranate, iced tea raspberry, iced tea tropical, incense essence indian curry, indian spice, irish coffee, irish cream, isotonic, italian licorice liqueur, italian liqueur, italian salami, italian seasoning, italian spice, jackfruit, jalapeño, jalapeño cheddar cheese, jalapeño pickled, jamaica, jamaica rum, jamaica rum punch, jamaican jerk, jamaican punch, jasmine, jasmine bouquet, jasmine tea, jelly bean, jerk seasoning, jicama, juicy lemon, juicy orange, juicy peach, juicy pear, kaffir lime, kahlua, kalamata olive, ketchup, ketchup curry, ketchup spice, key lime extract key lime, key lime pie, key lime tequila, kimchi, kirschwasser, kiwi, kiwi apple pie, kiwi boysenberry, kiwi grape, kiwi lemon lime, kiwi lime, kiwi macadamia nut, kiwi passion fruit, kiwi peach berry, kiwi peach, kiwi pineapple, kiwi punch, kiwi raspberry, kiwi strawberry, kiwime, kumquat, kumquat lime, kumquat orange, lactic ferment, lamb, lard, lavender, lavender honey, lavender vanilla, lemon extract lemon, lemon blossom, lemon bouquet, lemon custard, lemon dream, lemon filling, lemon ginger, lemon honey, lemon juice, lemon lime, lemon lime orange, lemon meyer, lemon oil, lemon peel, lemon pie, lemon pink, lemon punch, lemon raspberry, lemon rind, lemon tea, lemon vanilla, lemon zest, lemonade, lemonade pink, lemongrass, licorice, lime, lime blossom, lime chili, lime fresh, lime key, lime lemon, lime mint, lime spice, lime sweet citrus, lime tequila, lime zest, lingonberry, liver, lobster, loganberry, lollipop, lollipop blue raspberry, lollipop cherry, lollipop grape, lollipop lemon, lollipop strawberry, lotus bean, lychee, lychee bouquet, lychee coconut, lychee nut, macadamia nut, macapuno, macaron french, macaroon, mace, madagascar vanilla, madeira, mai tai, malt, malt chocolate, malt vanilla, malted milk, mamey, mandarin orange, mandarin tangerine, mango, mango barbecue, mango cream, mango fruit, mango chili, mango margarita, mango passion fruit, mango pineapple, mango punch, mango spice, mangosteen, manjar, maple extract maple, maple barbecue, maple butter, maple nut, maple pecan, maple praline, maple walnut, maraschino cherry, margarine, margarita, margarita blood orange, margarita mango, margarita strawberry, marionberry, marsala type, marshmallow, marshmallow toasted, mascarpone cheese, masking agent matcha, matcha cream, matcha mint, mayonnaise, meat, meat cheese blend, melon, melon cucumber, melon lychee, menthol, mesquite, mesquite smoke, mexican chocolate, mexican coffee, mexican coffee liqueur, mexican spice, mexican vanilla, mexicana oil, milk, milk chocolate, milk evaporated, milk sweetened condensed, milky chocolate, milky vanilla, mint extract mint, mint chocolate, mint mixed, mint vanilla, miso soup, mixed apple, mixed berry, mixed melon, mixed seafood, mixed tropical fruit, mixed vegetable, mixed wild berry, mocha, mocha almond, mocha almond fudge, mocha cafe, mocha cinnamon, mocha cinnamon almond, mocha coffee, mocha dutch chocolate, mocha white chocolate, mojito, molasses, monterey jack cheese, mortadella, mudslide, mushroom, mushroom portobello, mushroom sautéed, musk melon, mustard, mustard dijon, mustard honey, mustard hot, nacho cheese, naranjilla, nectaberry, nectarine, neufchatel cheese, nondairy cheese, nondairy creamer, nut, nut almond, nut brazil, nut butter, nut cashew, nut honey, nut honey roasted, nut macadamia, nut mixed, nut peanut, nut roasted chestnut, nutmeg, nutmeg spice, oak, oat, oat toasted, oatmeal, oatmeal apple cinnamon, oatmeal brown sugar, oatmeal cinnamon, oatmeal cookie, oatmeal enhancer, oatmeal honey, oatmeal maple, oatmeal maple brown sugar, oatmeal raisin, olive, olive oil, olive oil virgin, onion, onion caramelized, onion fried, onion green, onion maui, onion red, onion sauteed, onion toasted, onion minced, onion raw, onion white, oolong, orange extract orange, orange banana vanilla, orange bitter, orange blood, orange blossom, orange chocolate, orange cream, orange hawaiian, orange ice, orange juice, orange kiwi, orange lemon, orange lemon lime, orange lime, orange liqueur, orange mandarin, orange mango, orange naval, orange oil, orange passion fruit, orange peach, orange peel, orange pineapple, orange rind, orange spiced, orange sweet, orange tangerine, orange temple, orange thirst quencher, orange valencia, orange vanilla, orange whipped, orange zest, orange zest bitter, oregano, oriental, oyster, pad thai, pancake, pandan, papaya, papaya mango, parmesan cheese, parmesan garlic, parsley, passaberry, passion citrus, passion fruit, passion fruit banana, passion fruit cream, passion fruit mango, passion fruit strawberry, passion fruit tea, passion fruit vanilla, pea, peach, peach blossom, peach bouquet, peach brandy, pear cider, peach cream, peach fuzzy, peach mango, peach mixed, peach n′ berry, peach n′ cream, peach orange, peach passion fruit raspberry, peach roasted, peach spice, peach white, peaconut, peanut, peanut butter, peanut butter chocolate, peanut cajun, peanut honey roasted, peanut praline, peanut roasted, pear, pear bartlett, pear blossom, pear prickly, pecan, pecan butter, pecan candied, pecan cinnamon, pecan praline, pepita, pepita roasted, pepper, pepper black, pepper hot sauce, pepper red hot, pepper white, peppermint extract peppermint, peppermint chocolate, peppermint cream, peppermint oil, peppermint vanilla, pepperoni, pepperoni spicy, pepperoncini, persimmon, pesto, pesto parmesan, pfeffernusse, pho, pickle, pickle dill, pickle sweet, pico de gallo, pie apple, pie crust, pie pecan, pie pumpkin, pimento, piña colada, pinana, pine, pine bouquet, pine nut, pineapple, pineapple banana, pineapple chili, pineapple cider, pineapple coconut, pineapple coconut bouquet, pineapple cream, pineapple fresh, pineapple juice, pineapple mint, pineapple orange, pineapple pear, pineapple roasted, pink champagne, pinto bean, pippin apple, pistachio, pistachio cream, pizza, pizza pepperoni, pizza vegetable, plum, plum dried, plumogranate, poblano, pomegranate, pomegranate acai, pomegranate blueberry, pomelo, popcorn, popcorn buttered, popcorn kettle, porcini mushroom, pork, pork liver, pork vegetarian, potato, potato baked, potato butter, potato chip, potato fried, potato herb mashed, potato pulp sweet, potato roasted, potato sweet, pound cake, praline, praline almond, praline chocolate, praline cream, praline maple, praline peanut, prawn, prickly pear, prune, pumpernickel, pumpkin, pumpkin cheesecake, pumpkin pie, pumpkin pie ice cream, pumpkin pie spice, pumpkin spice, punch, punch tropical, quince, quinine, quinoa, raisin, raisin bread, raisin cinnamon, raisin walnut, rambutan, ranch, raspaya, raspberry, raspberry black, raspberry blue, raspberry boysenberry, raspberry cheesecake, raspberry chocolate, raspberry cream, raspberry grape, raspberry lemonade, raspberry lime, raspberry liqueur, raspberry lychee, raspberry pie, raspberry pineapple, raspberry spice, raspberry tamarind, raspberry tart, raspberry vanilla, raspberry yogurt, red and green chili, red apple, red bean, red bell pepper, red bell pepper roasted, red cherry, red chili, red currant, red date, red hot cinnamon, red pepper sauce, red sour cherry, red wine, red wine relish sweet, rhubarb, rhubarb strawberry, rice, rice milk, rice pudding, rice spanish, riesling, ripe banana, ripe cherry, ripe mango, ripened peach, roast beef, roasted, roasted almond, roasted cashew, roasted chicken, roasted garlic, roasted grain, roasted onion, roasted peanut, roasted pork, rompope, root beer, root beer float, rose, rose oil, rosemary extract rosemary, rosewater, rum, rum brandy, rum cognac, rum cola, rum dark, rum raisin, rumchata, russian beer, rye, s'more, saffron, salmon smoked, salsa, salsa chile de arbol, salsa chipotle, salsa fresca, salsa hot, salsa mesquite, salsa mild, salsa verde, salt & pepper, salt & vinegar, salted butter, sambuca, sangria, sansho, sarsaparilla, sassafras, sausage, sausage beer, sausage hot, sausage italian, sauternes wine, savory, scallop, scampi, scandinavian sweet dough, seafood, seafood mixed, seasoning mixed, seaweed type, semi-sweet chocolate, serrano, sesame, sesame black, sesame oil, sesame roasted, sesame seed, sesame toasted, shawarma, sherry cream, sherry, sherry rum, sherry wine, shish kabob, shiso, shrimp, shrimp scampi, smoke, smoke hickory, smoke mesquite, smoke oak, smoke pecan wood, smoky bacon, sour cream, sour cream & onion, sour cherry, sour lemon, sour milk, sourdough, southwest, soy sauce, soy type, spanish rice, spanish rice spice, spearmint extract spearmint, spearmint cream, spice, spice apple, spice blend, spice rum, spice tea, spicy hot, spinach, spumoni, sriracha type, sriracha barbecue type, sriracha honey type, sriracha maple type, starfruit, steak, steak grilled, steak medium, steak rare, steak well-done, steak seasoning, strapple, strawberry, strawberry banana, strawberry candy, strawberry cheesecake, strawberry chocolate, strawberry cream, strawberry hibiscus, strawberry kiwi, strawberry kiwi tangerine, strawberry lemonade, strawberry limeade, strawberry malt, strawberry margarita, strawberry milkshake, strawberry n′ cream, strawberry oil, strawberry passion fruit, strawberry pie, strawberry raspberry, strawberry shortcake, strawberry sweet, strawberry waffle, strawberry watermelon, strawberry white chocolate, strawberry yogurt, sugar brown, sugar burnt, sugar cookie, sugar green, sugar maple brown, sweet bread, sweet butter, sweet cherry, sweet cinnamon butter, sweet corn, sweet cream, sweet dough, sweet pickle, sweet n′ sour, sweet n′ sour spice, sweet n′ spicy, sweet potato, sweet potato fried, sweet potato pie, sweet potato pulp, sweet red bell pepper, sweet red pepper roasted, sweet roll danish, sweetened condensed milk, sweetener enhancer syrup maple, szechwan, szechwan hot, szechwan mild, taco, taco nacho spice, taco spicy, tahini express, tallow, tamarind, tamarindo, tangelo, tangerine, tangerine clementine, tangerine fresh, tangerine orange, taro, taro vanilla, tarragon, tartaric acid solution tea assam, tea black, tea green, tea hibiscus, tea iced, tea lemon, tea orange, tea raspberry, tea tropical, tequila, tequila chili, tequila lime, tequila sunrise, teriyaki, thai curry, thai spice, thai tea, tiramisu, toasted coconut, toasted corn, toasted frito type, tobacco, toffee, toffee english, tom yam kung, tomato, tomato basil, tomato juice, tomato ripe, tomato soup, tomato sun-dried, tonic, tortilla, tortilla flame roasted, triple see, tropical, tropical bouquet, tropical fruit, tropical fruit punch, tropical lemon, tropical lime, tropical paradise, tropical punch, tropical punch blue, truffle, truffle black, tuna, turkey, turkey fried, turkey honey roasted, turkey roasted, tutti frutti, ube, ume, vanilla, vanilla almond, vanilla bavarian, vanilla bean, vanilla bourbon, vanilla butter, vanilla butter nut, vanilla cake, vanilla cappuccino, vanilla cinnamon, vanilla coffee, vanilla cola, vanilla cookie, vanilla cream, vanilla custard, vanilla french, vanilla ice cream, vanilla irish creme, vanilla malt, vanilla nut, vanilla old fashioned, vanilla pudding, vanilla rum, vanilla shake, vanilla spice, vanilla syrup, vanilla tahitian, vanilla yogurt, vegetable roasted, vegetable mixed, vegetable soup, vegetarian bacon, vegetarian beef, vegetarian broth, vegetarian chicken, vegetarian ham, vegetarian pork, vegetarian sausage, vermouth, vinegar, vinegar apple cider, vinegar balsamic, vinegar malt, vinegar red wine, vinegar rice, vinegar white wine, waffle, waffle maple, walnut, walnut banana, walnut black, walnut bread, wasabi, watermelon, watermelon bouquet, watermelon candy, watermelon cherry, watermelon fresh, watermelon ginger, watermelon lime, watermelon strawberry, wheat, wheat bran, wheat germ, wheat honey, whiskey, white cake, white chocolate, white chocolate macadamia, white wine, wild berry, wild berry honey, wild cherry, wine, wine burgundy, wine cabernet, wine mulled, wine red, wine sweet, wine white, wintergreen, worcestershire, yam, yam brown sugar, yeast, yellow cake, yogurt, yogurt greek, yuca, yumberry, yuzu, za'atar, and zucchini.

In some preferred embodiments, flavors are organic flavors and/or natural flavors. In some preferred embodiments, flavors are sweet flavors. In some preferred embodiments, flavors are fruit flavors. In further preferred embodiments, flavors are berry flavors. In some preferred embodiments, between 1 and 5, between 1 and 4, or between 2 and 4 different flavors are used. In some preferred embodiments, 2, 3, or 4 different flavors are used. In some preferred embodiments, all flavors used are organic flavors and/or natural flavors, sweet flavors, and fruit and/or berry flavors. In other embodiments, one or more flavors is non-naturally occurring. In some preferred embodiments, each flavor is present in between about 0.1 and about 2.0 wt %, or between about 0.2 and about 1.5 wt %. In some preferred embodiments, each flavor is present in an amount of about 0.1 wt %, about 0.2 wt %, about 0.3 wt %, about 0.4 wt %, about 0.5 wt %, and so forth to about 2.0 wt %. In some preferred embodiments, each flavor is present in an amount of about 0.2 wt %, about 0.4 wt %, and about 1.5 wt %.

In some embodiments, the flavors in a disclosed formulation provide sweetness and/or flavor enhancement. In some embodiments, the flavors in a disclosed formulation provide flavor masking for one or more other, undesired flavors of one or more compounds or compositions in the formulation. In some preferred embodiments, the sweetness and/or flavor enhancement blocks the bitterness of one or more of the compounds in a kanna composition and/or in a synergistic plant composition.

In some embodiments, the disclosed formulation comprises a caramel base, such as an organic signature enhancer caramel base. In some preferred embodiments, the flavors are combined with the caramel base. In some preferred embodiments, the caramel base is present in an amount of between 0.05 wt % and 0.5 wt %. In some preferred embodiments, the caramel base is present in an amount of 0.15 wt %.

Colorants and/or dyes and/or pigments may be added to disclosed formulations in some embodiments. Suitable colorants and/or dyes and/or pigments may include, but not be limited to, colors such as e.g., white, black, yellow, blue, green, pink, red, orange, violet, indigo, brown, and combinations thereof, pigments such as, e.g., Timica Extra Large Sparkles, titanium dioxide and chromium oxide greens, ultramarine blues and pinks and ferric oxides. In some embodiments, a colorant in a disclosed formulation is a natural ingredient. In some embodiments, a colorant in a disclosed formulation is non-naturally occurring. In some embodiments, a colorant (or combination of colorants) used in a disclosed formulation comprises a plant extract. In some embodiments, a colorant (or combination of colorants) used in a disclosed formulation comprises a vegetable extract. In some embodiments, the extract is any of a spinach, blueberry, carrot, beet, red cabbage, turmeric, annatto seed, saffron, paprika, tomato, grape, cherry, black carrot, pumpkin, sweet potato, elderberry, spirulina, bell pepper, sweet potato, kale, hibiscus flower, matcha, parsley, mint, pea, cucumber, aronia, blackberry, raspberry, and cranberry extract, including combinations thereof. Colorants and/or dyes and/or pigments may be present, individually or in total (if more than one colorant and/or dye and/or pigment is included), in disclosed formulations in an amount ranging from about 0.01 wt % to about 5 wt % (calculated as the total weight of colorants and/or dyes and/or pigments in the formulation divided by the total weight of the formulation). In some embodiments, a disclosed formulation does not comprise a colorant. In some embodiments, a disclosed formulation does not comprise a dye. In some embodiments, a disclosed formulation does not comprise a pigment.

Antioxidants may be added to disclosed formulations in some embodiments. Suitable antioxidants may include, but not be limited to, natural antioxidants such as tocopherol and tocopherol acetate. Antioxidants may be present, individually or in total (if more than one antioxidant is included), in disclosed formulations in an amount ranging from about 0.1 wt % to about 5 wt % (calculated as the total weight of antioxidants in the formulation divided by the total weight of the formulation). In some embodiments, the antioxidant is a natural ingredient. In some embodiments, the antioxidant is non-naturally occurring. In some embodiments, a disclosed formulation does not comprise an antioxidant.

The formulations of the disclosure may contain a binder that could contribute to the rheology of the formulation and to the feel of the formulation in the oral cavity. Suitable binders include, without limitations, polyvinylpyrrolidone (PVP), marine colloids, carboxyvinyl polymers, starches, cellulosic polymers such as hydroxyethylcellulose, carboxymethylcellulose (carmellose), hydroxypropyl methyl cellulose, hydroxyethyl-propylcellulose, hydroxybutyl methyl cellulose, and salts thereof (e.g., carmellose sodium), natural gums such as karaya, xanthan, carrageenans, gellan gum, locust bean gum, gum arabic and tragacanth, chitosan, colloidal magnesium aluminum silicate, and colloidal silica. Binders may be present, individually or in total (if more than one binder is included), in disclosed formulations in an amount ranging from about 0.01 wt % to about 5 wt % (calculated as the total weight of binders in the formulation divided by the total weight of the formulation). In some embodiments, the binder is a natural ingredient. In some embodiments, the binder is non-naturally occurring. In some embodiments, a disclosed formulation does not comprise a binder.

Solvents that may be included in disclosed formulations may include, without limitations, water, polyhydric alcohols (e.g., glycerin), 1,3-butylene glycol, propylene glycol, hexylene glycol, propane diol, ethylene glycol, diethylene glycol, dipropylene glycol, diglycerin, sorbitol, other sugars which are liquid at room temperature, water-soluble alkoxylated nonionic polymers such as polyethylene glycol, and combinations thereof. Solvents may be present, individually or in total (if more than one solvent is included), in the formulation in an amount ranging from about 0.1 wt % to about 95 wt % (calculated as the total weight of solvents in the formulation divided by the total weight of the formulation). In some embodiments, the solvent is a natural ingredient. In some embodiments, the solvent is non-naturally occurring. In some embodiments, a disclosed formulation does not comprise a solvent.

Humectant(s), such as low molecular weight polyethylene glycol (e.g., PEG6-PEG12), may be present, individually or in total (if more than one humectant is included), in the formulation in an amount of up to about 10 wt %, up to about 5 wt %, up to about 3 wt %, up to about 1 wt %, or up to about 0.1 wt % (calculated as the total weight of humectants in the formulation divided by the total weight of the formulation). In some embodiments, the humectant is a natural ingredient. In some embodiments, the humectant is non-naturally occurring. In some embodiments, a disclosed formulation does not comprise a humectant.

Disclosed formulations may include a sweetener capable of providing a palatable and pleasurable factor to the user, and/or capable of masking undesirable flavors present in the dosage form. Exemplary sweeteners that may be included in disclosed formulations may include, but not be limited to, one or more artificial sweeteners, one or more natural sweeteners, one or more soluble fibers (e.g., for a sugar-free formulation), or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like. Examples of soluble fibers that can be incorporated into the formulations, alone or in combination with other sweeteners, may include, but not be limited to, soluble fibers from tapioca, fruit (e.g., pears, nectarines, apples, and the like), starch, nuts (such as oats, hazelnuts, barley and the like), and legume sources. Sweeteners may be present, individually or in total (if more than one sweetener is included), in the formulation in an amount ranging from about 0.1 wt % to about 20 wt % (calculated as the total weight of sweeteners in the formulation divided by the total weight of the formulation). In some embodiments, the sweetener is a natural ingredient. In some embodiments, the sweetener is non-naturally occurring. In some embodiments, a disclosed formulation does not comprise a sweetener. In some embodiments, a disclosed formulation does not comprise a sugar.

Surfactants may be incorporated in disclosed formulations. The surfactants that can be included in the formulation may be anionic, nonionic, or amphoteric compounds. Suitable examples of anionic surfactants are one or more of higher alkyl sulfates such as potassium or sodium lauryl sulfate, higher fatty acid monoglyceride monosulfates, such as the salt of the monosulfated monoglyceride of hydrogenated coconut oil fatty acids, alkyl sulfonates such as sodium dodecyl benzene sulfonate, higher fatty sulfoacetates, higher fatty acid esters of 1,2 dihydroxypropane sulfonate. Examples of water soluble nonionic surfactants are condensation products of ethylene oxide with various hydrogen-containing compounds that are reactive therewith and have long hydrophobic chains (e.g., aliphatic chains of about 12 of 20 carbon atoms), which condensation products contain hydrophilic polyoxyethylene moieties, such as condensation products of poly (ethylene oxide) with fatty acids, fatty alcohols, fatty amides and other fatty moieties, and with propylene oxide and polypropylene oxides, e.g., Pluronic materials such as Pluronic F127. Exemplary suitable alkyl polyglycoside (APG) surfactant(s) that may be used in the formulation may comprise APG C8-C10, APG C10-C16, decyl glucoside, coco-glucoside, anionic APG carboxylate, sodium lauryl glucose carboxylate, lauryl glucoside, D-glucopyranose (oligomeric, CIO-16 glycosides, carboxymethyl ethers, sodium salts), C12-C16 fatty alcohol glycoside, and combinations thereof. Exemplary APG surfactant(s) that may be used may have an industry designation of Plantaren® 2000 N UP/MB, Plantapon® LGC Sorb, Plantaren® 1200 N UP/MB, and Plantaren® 818 UP/MB. Surfactants may be present, individually or in total (if more than one surfactant is included) in the formulation in an amount ranging from about 0.01 wt % to about 10 wt % (calculated as the total weight of surfactants in the formulation divided by the total weight of the formulation). In some embodiments, the surfactant is a natural ingredient. In some embodiments, the surfactant is non-naturally occurring. In some embodiments, a disclosed formulation does not comprise a surfactant.

Exemplary gelling agent(s) that may be issued in disclosed formulations may comprise pectins, starches, and gelatin forms derived from animals or from plants (e.g., pork gelatin) or a different gelling agent that would be suitable for consumption by individuals who do not consume meat products (e.g., vegetarians, vegans). The pectin in the formulation may include, e.g., high methoxyl pectin, low methoxyl pectin, or a combination thereof. In some embodiments, the pectin is amidated pectin. In other embodiments, the pectin is non-amidated pectin. In certain embodiments, the pectin is a combination of amidated pectin and non-amidated pectin. The gelatin in the formulation may include Type A gelatin, Type B gelatin, a hide or skin gelatin (e.g., calf skin, pig skin) and/or a bone gelatin (e.g., calf bone, pig bone) used alone or in combination. Gelling agent(s) may be present, individually or in total (if more than one gelling agent is included) in the formulation in an amount ranging from about 0.1 wt % to about 20 wt % (calculated as the total weight of gelling agents in the formulation divided by the total weight of the formulation). In some embodiments, the gelling agent is a natural ingredient. In some embodiments, the gelling agent is non-naturally occurring. In some embodiments, a disclosed formulation does not comprise a gelling agent.

In some embodiments, disclosed formulations may comprise a preservative. Preservatives can be used to inhibit microbial growth or increase stability of the formulation, thereby prolonging the shelf life of the formulation. Suitable preservatives are known in the art and include EDTA, EGTA, benzalkonium chloride or benzoic acid or benzoates (e.g., sodium benzoate), vitamin A, vitamin C (ascorbic acid), citric acid, vitamin E, and tocopherol. In some embodiments, the preservative is a natural ingredient. In some embodiments, the preservative is non-naturally occurring. In some embodiments, a disclosed formulation does not comprise a preservative.

In some embodiments, a disclosed empathogenic botanical formulation does not comprise a flavoring agent, a sweetener, a colorant, a dye, a pigment, an antioxidant, a binder, a solvent, a humectant, a viscosity modifier, a solubilizer, a complexing agent, a preservative, a pH adjusting agent, an opacifier, a surfactant, a gelling agent, or an effervescence material. In embodiments, a disclosed empathogenic botanical formulation does not comprise a flavoring agent. In embodiments, a disclosed empathogenic botanical formulation does not comprise a sweetener. In embodiments, a disclosed empathogenic botanical formulation does not comprise an artificial sweetener. In embodiments, a disclosed empathogenic botanical formulation does not comprise a sugar. In embodiments, a disclosed empathogenic botanical formulation does not comprise a colorant. In embodiments, a disclosed empathogenic botanical formulation does not comprise a dye. In embodiments, a disclosed empathogenic botanical formulation does not comprise a pigment. In embodiments, a disclosed empathogenic botanical formulation does not comprise an antioxidant. In embodiments, a disclosed empathogenic botanical formulation does not comprise a binder. In embodiments, a disclosed empathogenic botanical formulation does not comprise a synthetic binder. In embodiments, a disclosed empathogenic botanical formulation does not comprise an elastomer. In embodiments, a disclosed empathogenic botanical formulation does not comprise a solvent. In embodiments, a disclosed empathogenic botanical formulation does not comprise a humectant. In embodiments, a disclosed empathogenic botanical formulation does not comprise a viscosity modifier. In embodiments, a disclosed empathogenic botanical formulation does not comprise a solubilizer. In embodiments, a disclosed empathogenic botanical formulation does not comprise a complexing agent. In embodiments, a disclosed empathogenic botanical formulation does not comprise a preservative. In embodiments, a disclosed empathogenic botanical formulation does not comprise a pH adjusting agent. In embodiments, a disclosed empathogenic botanical formulation does not comprise an opacifier. In embodiments, a disclosed empathogenic botanical formulation does not comprise a surfactant. In embodiments, a disclosed empathogenic botanical formulation does not comprise a gelling agent. In embodiments, a disclosed empathogenic botanical formulation does not comprise an effervescence material.

In some embodiments, a disclosed formulation also can be defined by one or more characteristics and/or ingredients which it does not provide or comprise, for example any one or more of: no sugar; no calories; no GMO; no soy; no nuts; no animal products; no caffeine; no artificial sweeteners; no preservatives; no plastic; non-hallucinogenic; non-drowsy; non-addictive; non-intoxicating; and non-pharmaceutical.

In some embodiments, a disclosed empathogenic botanical formulation comprising a kanna composition; a synergistic plant composition; and a binding composition.

In some embodiments, provided is an empathogenic botanical formulation, comprising:
a. a kanna composition comprising:
   i. a first kanna extract;
   ii. a second kanna extract; and
   iii. a fermented kanna plant powder, an unfermented kanna plant powder, or a mixture;
b. a synergistic plant composition;
c. a binding composition;
d. at least one flavoring agent; and
e. at least one coloring agent.

In some embodiments, provided is an empathogenic botanical formulation, comprising:
a. a kanna composition comprising:
   i. a kanna extract; and
   ii. a fermented kanna plant powder, an unfermented kanna plant powder, or a mixture;
b. a synergistic plant composition;
c. a binding composition; and
d. at least one flavoring agent.

In some embodiments, the kanna composition consists of a kanna extract and a kanna plant powder. In some embodiments, the kanna composition consists essentially of a kanna extract and a kanna plant powder. In some embodiments, the kanna composition consists of a kanna extract and a fermented kanna plant powder. In some embodiments, the kanna composition consists essentially of a kanna extract and a fermented kanna plant powder. In embodiments, the kanna composition consists of a kanna extract and an unfermented kanna plant powder. In embodiments, the kanna composition consists essentially of a kanna extract and an unfermented kanna plant powder. In embodiments, the kanna composition consists of a kanna extract and a kanna plant powder that is a mixture of fermented kanna plant powder and unfermented kanna plant powder. In embodiments, the kanna composition consists essentially of a kanna extract and a kanna plant powder that is a mixture of fermented kanna plant powder and unfermented kanna plant powder.

In some embodiments, provided is an empathogenic botanical formulation, comprising:
a. a kanna composition;
b. a synergistic plant composition comprising:
   i. Acacia senegal plant powder;
   ii. Acmella oleracea plant powder;
   iii. Saussurea involucrata plant powder;
   iv. Lavandula angustifolia plant powder; and
   v. Mentha spicata extract;
c. a binding composition; and
d. at least one flavoring agent.

In some embodiments, the synergistic plant composition consists of (i) Acacia senegal plant powder; (ii) Acmella oleracea plant powder; (iii) Saussurea involucrata plant powder; (iv) Lavandula angustifolia plant powder; and (iv) mint extract. In some embodiments, the synergistic plant composition consists essentially of (i) Acacia senegal plant powder; (ii) Acmella oleracea plant powder; (iii) Saussurea involucrata plant powder; (iv) Lavandula angustifolia plant powder; and (iv) mint extract.

In some embodiments, provided is an empathogenic botanical formulation, comprising:
a. a kanna composition;
b. a synergistic plant composition; and
c. a binding composition comprising:
   i. agar;
   ii. vegetable glycerin; and
   iii. distilled water; and
d. at least one flavoring agent.

In embodiments, the binding composition consists of agar, vegetable glycerin, and distilled water. In embodiments, the binding composition consists essentially of agar, vegetable glycerin, and distilled water.

In some embodiments, provided is an empathogenic botanical formulation, comprising:
a. a kanna composition comprising:
   i. a kanna extract; and
   ii. a fermented kanna plant powder, an unfermented kanna plant powder, or a mixture;
b. a synergistic plant composition comprising:
   i. Acacia senegal plant powder;
   ii. Acmella oleracea plant powder;
   iii. Saussurea involucrata plant powder;
   iv. Lavandula angustifolia plant powder; and
   v. Mentha spicata extract; and
c. a binding composition comprising:
   i. agar;
   ii. vegetable glycerin;
   iii. distilled water; and
d. at least one flavoring agent.

In some embodiments, a disclosed empathogenic botanical formulation consists of a kanna composition; a synergistic plant composition; and a binding composition. In some embodiments, provided is an empathogenic botanical formulation, consisting of:
a. a kanna composition consisting of:
   i. a kanna extract; and
   ii. a fermented kanna plant powder, an unfermented kanna plant powder, or a mixture;
b. a synergistic plant composition; and
c. a binding composition; and
d. at least one flavoring agent.

In some embodiments, provided is an empathogenic botanical formulation, consisting of:
a. a kanna composition;
b. a synergistic plant composition consisting of:
   i. Acacia senegal plant powder;
   ii. Acmella oleracea plant powder;
   iii. Saussurea involucrata plant powder;
   iv. Lavandula angustifolia plant powder; and
   v. Mentha spicata extract;
c. a binding composition; and
d. at least one flavoring agent.

In some embodiments, provided is an empathogenic botanical formulation, consisting of:
a. a kanna composition;
b. a synergistic plant composition; and
c. a binding composition consisting of:
   i. agar;
   ii. vegetable glycerin;
   iii. distilled water; and
d. at least one flavoring agent.

In some embodiments, provided is an empathogenic botanical formulation, consisting of:
a. a kanna composition consisting of:
   i. a kanna extract; and
   ii. a fermented kanna plant powder, an unfermented kanna plant powder, or a mixture;
b. a synergistic plant composition consisting of:
   i. Acacia senegal plant powder;
   ii. Acmella oleracea plant powder;
   iii. Saussurea involucrata plant powder;
   iv. Lavandula angustifolia plant powder; and
   v. Mentha spicata extract; and
c. a binding composition consisting of:
   i. agar;
   ii. vegetable glycerin;
   iii. distilled water; and
d. at least one flavoring agent.

In some embodiments, a disclosed empathogenic botanical formulation consists essentially of a kanna composition; a synergistic plant composition; and a binding composition. In some embodiments, provided is an empathogenic botanical formulation, consisting essentially of:
a. a kanna composition consisting essentially of:
   i. a kanna extract; and
   ii. a fermented kanna plant powder, an unfermented kanna plant powder, or a mixture;
b. a synergistic plant composition;
c. a binding composition; and
d. at least one flavoring agent.

In some embodiments, provided is an empathogenic botanical formulation, consisting essentially of:
a. a kanna composition;
b. a synergistic plant composition consisting essentially of:
   i. Acacia senegal plant powder;
   ii. Acmella oleracea plant powder;
   iii. Saussurea involucrata plant powder;
   iv. Lavandula angustifolia plant powder; and
   v. Mentha spicata extract;
c. a binding composition; and
d. at least one flavoring agent.

In some embodiments, provided is an empathogenic botanical formulation, consisting essentially of:
a. a kanna composition;
b. a synergistic plant composition; and
c. a binding composition consisting essentially of:
   i. agar;
   ii. vegetable glycerin;
   iii. distilled water; and
d. at least one flavoring agent.

In some embodiments, provided is an empathogenic botanical formulation, consisting essentially of:
a. a kanna composition consisting essentially of:
   i. a kanna extract; and
   ii. a fermented kanna plant powder, an unfermented kanna plant powder, or a mixture;
b. a synergistic plant composition consisting essentially of:
   i. Acacia senegal plant powder;
   ii. Acmella oleracea plant powder;
   iii. Saussurea involucrata plant powder;
   iv. Lavandula angustifolia plant powder; and
   v. Mentha spicata extract; and
c. a binding composition consisting essentially of:
   i. agar;
   ii. vegetable glycerin;
   iii. distilled water; and
d. at least one flavoring agent.

In some embodiments, provided is an empathogenic botanical formulation, comprising:
a. a kanna composition;
b. a synergistic plant composition comprising:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract;
c. a binding composition; and
d. at least one flavoring agent.

In some embodiments, the synergistic plant composition consists of (i) Acacia senegal plant powder; (ii) Acmella oleracea plant powder; (iii) Saussurea involucrata plant powder; (iv) rose petal flower; (v) Zingiber officinale root powder; (vi) Lavandula angustifolia plant powder; and (vii) mint extract. In some embodiments, the synergistic plant composition consists essentially of (i) Acacia senegal plant powder; (ii) Acmella oleracea plant powder; (iii) Saussurea involucrata plant powder; (iv) rose petal flower; (v) Zingiber officinale root powder; (vi) Lavandula angustifolia plant powder; and (vii) mint extract.

In some embodiments, provided is an empathogenic botanical formulation, comprising:
a. a kanna composition;
b. a synergistic plant composition; and
c. a binding composition comprising:
   i. guar gum;
   ii. vegetable glycerin; and
   iii. distilled water; and
d. at least one flavoring agent.

In some embodiments, the binding composition consists of guar gum, vegetable glycerin, and distilled water. In some embodiments, the binding composition consists essentially of guar gum, vegetable glycerin, and distilled water.

In some embodiments, provided is an empathogenic botanical formulation, comprising:
a. a kanna composition comprising:
   i. a kanna extract; and
   ii. a fermented kanna plant powder, an unfermented kanna plant powder, or a mixture;
b. a synergistic plant composition comprising:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract; and
c. a binding composition comprising:
   i. guar gum;
   ii. vegetable glycerin;
   iii. distilled water; and
d. at least one flavoring agent.

In some embodiments, a disclosed empathogenic botanical formulation consists of a kanna composition; a synergistic plant composition; and a binding composition. In some embodiments, provided is an empathogenic botanical formulation, consisting of:
a. a kanna composition consisting of:
   i. a kanna extract; and
   ii. a fermented kanna plant powder, an unfermented kanna plant powder, or a mixture;
b. a synergistic plant composition; and
c. a binding composition; and
d. at least one flavoring agent.

In some embodiments, provided is an empathogenic botanical formulation, consisting of:
a. a kanna composition;
b. a synergistic plant composition consisting of:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract;
c. a binding composition; and
d. at least one flavoring agent.

In some embodiments, provided is an empathogenic botanical formulation, consisting of:
a. a kanna composition;
b. a synergistic plant composition; and
c. a binding composition consisting of:
   i. guar gum;
   ii. vegetable glycerin;
   iii. distilled water; and
d. at least one flavoring agent.

In some embodiments, provided is an empathogenic botanical formulation, consisting of:
a. a kanna composition consisting of:
   i. a kanna extract; and
   ii. a fermented kanna plant powder, an unfermented kanna plant powder, or a mixture;
b. a synergistic plant composition consisting of:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract; and
c. a binding composition consisting of:
   i. guar gum;
   ii. vegetable glycerin;
   iii. distilled water; and
d. at least one flavoring agent.

In some embodiments, a disclosed empathogenic botanical formulation consists essentially of a kanna composition; a synergistic plant composition; and a binding composition. In some embodiments, provided is an empathogenic botanical formulation, consisting essentially of:
a. a kanna composition consisting essentially of:
   i. a kanna extract; and
   ii. a fermented kanna plant powder, an unfermented kanna plant powder, or a mixture;
b. a synergistic plant composition;
c. a binding composition; and
d. at least one flavoring agent.

In some embodiments, provided is an empathogenic botanical formulation, consisting essentially of:
a. a kanna composition;
b. a synergistic plant composition consisting essentially of:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract;
c. a binding composition; and
d. at least one flavoring agent.

In some embodiments, provided is an empathogenic botanical formulation, consisting essentially of:
a. a kanna composition;
b. a synergistic plant composition; and
c. a binding composition consisting essentially of:
   i. guar gum;
   ii. vegetable glycerin;
   iii. distilled water; and
d. at least one flavoring agent.

In some embodiments, provided is an empathogenic botanical formulation, consisting essentially of:
a. a kanna composition consisting essentially of:
   i. a kanna extract; and
   ii. a fermented kanna plant powder, an unfermented kanna plant powder, or a mixture;
b. a synergistic plant composition consisting essentially of:
   i. Acacia senegal plant powder;
   ii. Acmella oleracea plant powder;
   iii. Saussurea involucrata plant powder;
   iv. rose petal flower;
   v. Zingiber officinale root powder;
   vi. Lavandula angustifolia plant powder; and
   vii. Mentha spicata extract; and
c. a binding composition consisting essentially of:
   i. guar gum;
   ii. vegetable glycerin;
   iii. distilled water; and
d. at least one flavoring agent.

In some embodiments, provided is an empathogenic botanical formulation, comprising:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material.

In some embodiments, provided is an empathogenic botanical formulation, consisting of:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material.

In some embodiments, provided is an empathogenic botanical formulation, consisting essentially of:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material.

In some embodiments, provided is an empathogenic botanical formulation, comprising:
a. a kanna composition comprising:
   i. a first kanna extract;
   ii. a second kanna extract; and
   iii. a fermented kanna plant powder, an unfermented kanna plant powder, or a mixture; and
b. a synergistic plant composition; and
c. a binding composition comprising:
   i. guar gum;
   ii. vegetable glycerin; and
   iii. distilled water.

In some embodiments, provided is an empathogenic botanical formulation, consisting of:
a. a kanna composition comprising:
   i. a first kanna extract;
   ii. a fermented kanna plant powder, an unfermented kanna plant powder, or a mixture; and
b. a synergistic plant composition; and
c. a binding composition comprising:
   i. guar gum;
   ii. vegetable glycerin; and
   iii. distilled water.

In some embodiments, provided is an empathogenic botanical formulation, consisting essentially of:
a. a kanna composition comprising:
   i. a first kanna extract;
   ii. a second kanna extract; and
   iii. a fermented kanna plant powder, an unfermented kanna plant powder, or a mixture; and
b. a synergistic plant composition; and
c. a binding composition comprising:
   i. guar gum;
   ii. vegetable glycerin; and
   iii. distilled water.

In some embodiments, provided is an empathogenic botanical formulation, comprising:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material; and
c. a binding composition comprising:
   i. guar gum;
   ii. vegetable glycerin; and
   iii. distilled water.

In some embodiments, provided is an empathogenic botanical formulation, consisting of:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material; and
c. a binding composition comprising:
   i. guar gum;
   ii. vegetable glycerin; and
   iii. distilled water.

In some embodiments, provided is an empathogenic botanical formulation, consisting essentially of:
a. a kanna composition comprising:
   i. a first kanna extract;
   ii. a second kanna extract; and
   iii. a fermented kanna plant powder, an unfermented kanna plant powder, or a mixture; and
b. a synergistic plant composition comprising:
   i. Acacia senegal plant powder;
   ii. Acmella oleracea plant powder; and
   iii. any one or more of Acacia senegal plant material, Acmella oleracea plant material, Saussurea involucrata plant material, Lavandula angustifolia plant material, Mentha spicata extract, Zingiber officinale plant material, and Rosa sp. plant material; and
c. a binding composition comprising:
   i. guar gum;
   ii. vegetable glycerin; and
   iii. distilled water.

In embodiments, any of the fermented kanna plant powder, the unfermented kanna plant powder, or the mixture in examples above is a full-spectrum kanna extract, which may be an extract such as the kanna plant powder disclosed above with the characteristics represented by the exemplary certificate of analysis.

In embodiments, the kanna plant powder is a fermented kanna plant powder. In embodiments, the kanna plant powder is an unfermented kanna plant powder. In embodiments, the kanna plant powder is a mixture of fermented kanna plant powder and unfermented kanna plant powder. A mixture may comprise a fermented kanna plant powder and an unfermented kanna plant powder in any proportions, e.g., from about 1:1000 to about 1000:1, such as about 1:100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1.5, 1:1.25, 1:1.1, 1:1.05, and 1:1.01, and about 1.01:1, 1.05:1, 1.1:1, 1.25:1, 1.5:1, 2:1, 3:1, 4:1, 5:1, 6:1, 7:1, 8:1, 9:1, 10:1, 20:1, 30:1, 40:1, 50:1, 60:1, 70:1, 80:1, 90:1, and 100:1, including ratios in between. Mixtures also may comprise more than one fermented kanna plant powder and one unfermented kanna plant powder, as well as one fermented kanna plant powder and more than one unfermented kanna plant powder; including two or more, three or more, four or more, and five or more.

The pH of disclosed formulations may range from about 4 to about 10, from about 5 to about 9, from about 6 to about 8, or about 7. In some embodiments, the pH of a disclosed empathogenic botanical formulation is about 7. The pH of a formulation can be measured by methods known to those of skill in the art, for example by dissolving the formulation in water and using a pH meter equipped with a glass combined electrode. The pH of the formulation may be adjusted with a pH adjusting material such as, without limitations, citric acid, hydrochloric acid, sodium hydroxide, etc. The pH adjusting agent may be present, individually or in total (if more than one pH adjusting agent is included), in the formulation in an amount ranging from about 0.01 wt % to about 2 wt %, from about 0.1 wt % to about 1 wt %, or from about 0.2 wt % to about 0.5 wt % (calculated as the total weight of pH adjusting agent(s) in the formulation divided by the total weight of the formulation).

Conventionally, chew formulations and gummy formulations are heated to close to or above their melting points to facilitate forming discrete chews and gummies (e.g., by using silicone molds). However, in some embodiments, disclosed empathogenic botanical formulations can be produced without the direct application of heat during the manufacturing process. Accordingly, in embodiments, disclosed formulations have improved flavor, aroma, and/or texture characteristics owing to decreased thermal or heat-induced decomposition of fragile flavor, aroma, or texture compounds. In some embodiments, disclosed formulations have improved bioactivity owing to decreased thermal or heat-induced decomposition of bioactive compounds, such as kanna alkaloids, or other bioactive compounds in the synergistic plant composition.

In some embodiments, the disclosed empathogenic botanical formulations comprise only naturally occurring substances. In some embodiments, the disclosed empathogenic botanical formulations comprise at least one non-naturally occurring substance. In some embodiments, including in some preferred embodiments, wherein the disclosed empathogenic botanical formulation comprises only naturally occurring substances, the formulation (i.e., the combination), and/or the natural components thereof, have at least one markedly different characteristic compared to any naturally occurring counterparts in their natural state. Such markedly different characteristics may include, as non-limiting examples, the biological and pharmacological functions and activities, the chemical and physical properties, the functional and structural characteristics, and the structure and chemical and physical form.

### C. DOSE AND DOSAGE

The disclosed empathogenic botanical formulations are preferably formulated as a unit dosage form, each dosage containing an effective amount of the active ingredient(s), for example in the dosage amounts disclosed below. The term “unit dosage form” refers to a physically discrete unit suited as unitary dosages to be consumed by the individual, each unit containing a predetermined quantity of active material calculated to produce the desired empathogenic effect(s).

Unit dosage forms are often used for ease of administration and uniformity of dosage. Unit dosage forms can contain a single or individual dose or unit, a sub-dose, or an appropriate fraction thereof, of the empathogenic botanical formulation. Unit dosage forms include gummies, chews, and the like.

In some embodiments, a unit dosage form of a disclosed formulation comprises a total dose of kanna of, e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges. In some preferred embodiments, a unit dosage form of a disclosed formulation comprises a total dose of kanna of about 30 mg.

In embodiments wherein a disclosed formulation is used to create an empathogenic effect, dose and dosage may vary depending upon the general health, age, gender, and race of the individual, bioavailability, potential adverse systemic, regional, or local side effects, the presence of any disorders or diseases in the individual, and other factors that will be appreciated by those in the art (e.g., medical or familial history).

In general, dose amount, frequency, or duration may be increased or reduced, as indicated by the therapeutic outcome(s) or effect(s) desired, the beneficial outcome(s) or effect(s) desired, and/or by the specific subjective outcome(s) or effect(s) desired.

Those in the art will appreciate the factors that may influence the dosage, frequency, and timing required to provide an amount sufficient or effective for providing a desired effect, and to do so depending on the type of desired effect and to avoid or minimize adverse effects.

Dosage levels may differ from patient to patient, for individuals across time, and for different compositions and formulations, but shall be able to be determined with ordinary skill. Determination of appropriate dosing shall include not only the determination of single dosage amounts, but also the determination of the number and timing of doses, and the time(s) of day or time(s) preferable for administration.

In some embodiments, a disclosed formulation will be taken on an empty stomach. In some embodiments, a disclosed formulation will be taken up to three times per 24-hour period. In some embodiments, a disclosed formulation will not be taken right before bed. In some embodiments, a disclosed formulation will not be taken in conjunction with monoamine oxidase inhibitors (MAOIs), selective serotonin reuptake inhibitors (SSRIs), or serotonin-norepinephrine reuptake inhibitors (SNRIs). In some embodiments, a disclosed formulation will not be given to or taken by an individual under 18 years of age. In some embodiments, a disclosed formulation will not be taken by an individual while pregnant or nursing. In some embodiments, a disclosed formulation will not be given to or taken by an individual with a medical condition or an individual taking any prescription medications unless a healthcare provider is consulted.

In some embodiments, the disclosed formulations are administered together with one or more therapeutically beneficial activities, for example where such participation follows or is in conjunction with the administration, including breathwork and breathing exercises, meditation and concentration practices, focusing on an object or mantra, listening to music, physical exercise, yoga, stretching or bodywork, journaling, grounding techniques, positive self-talk, or engaging with a pet or animal.

In some embodiments, the disclosed formulations are administered together with one or more sessions of psychotherapy, for example where such psychotherapy precedes, follows, or is in conjunction with the administration.

The present disclosure further provides kits comprising the disclosed empathogenic botanical formulations. In some embodiments, the kits provide disclosed formulations in unit dosage form. Kits generally comprise suitable packaging. The kits may comprise one or more containers comprising any formulation described herein. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. For example, kits may be provided that contain sufficient dosages of a disclosed formulation for an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses and instructions for use and be packaged in quantities sufficient for storage at home or a retail location. The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

In some embodiments, the instructions direct an individual to consume the formulation by masticating (e.g., chewing) the formulation to promote mucosal absorption in the oral cavity rather than swallowing the formulation, to avoid undesirable gastrointestinal effects.

### D. “EMPATHOGENIC” AND OTHER SYNERGISTIC PSYCHOACTIVE, BENEFICIAL AND THERAPEUTIC EFFECTS

In some aspects, administration of a disclosed formulation to a subject, which shall be understood to include self-administration by an individual, results in an “empathogenic” effect.

An “empathogenic” effect will be known to those in the art, and herein also may refer to, as non-limiting examples, feelings: of deeper connection to oneself and others; of restoration and coherence of body, heart, mind and spirit; of head-heart harmony; of being in balance and healthy homeostasis; of joy and the highest form of well being; of the fullness of one's aliveness; of radiating from within, of the ability to positively affect everything around oneself; of elevated vibes; and of aliveness and lifted mood.

In general, the psychoactive and empathogenic effects of the disclosed formulations will affect the mind and mental processes, and have the ability to change mood and awareness, by lifting the mood and relaxing the mind, body and spirit while offering a solid dose of sustained grounded energy. In some embodiments, disclosed formulations uplift and ground at the same time. In some embodiments, disclosed formulations cause heightened cognition, mental acuity, and focus. In some embodiments, disclosed formulations activate full-spectrum aliveness.

In some embodiments, disclosed formulations support nervous, circulatory, immune, and digestive systems.

Although an empathogenic effect will be known in the art, it also will be appreciated that while the disclosed formulations may be considered a natural MDMA alternative, in some embodiments, including in some preferred embodiments, they will not be intended to, nor will under standard dosages and/or administration conditions such as disclosed herein, cause one to feel “high” or affect one's sobriety level.

As has been described for the disclosed formulations: “You'll feel here, feel clear, feel yourself.”

In some embodiments, disclosed empathogenic botanical formulations reduce stress or anxiety, and cause for example less everyday anxiety. In some embodiments, the reduction of stress or anxiety is assessed relative to a baseline that is defined by the individual's normal, everyday level of stress or anxiety when they have not been administered (or have not self-administered) a disclosed formulation. In some embodiments, an individual's level of stress or anxiety can be determined using known assessments and questionnaires, such as the Generalized Anxiety Disorder 7 (GAD-7) Scale, which is a self-reported questionnaire for screening and severity measuring of generalized anxiety disorder (Spitzer, et al. Arch Intern Med. 2006, 166(10), 1092-1097). In some embodiments, reducing an individual's stress or anxiety is effective for treating an anxiety disorder, such as generalized anxiety disorder, a phobia disorder, social anxiety, panic, end-of-life anxiety, anxiety associated with a terminal illness, cancer-related anxiety, post-traumatic stress disorder (PTSD), or adjustment disorder.

In some embodiments, disclosed empathogenic botanical formulations improve mood. In some embodiments, disclosed formulations provide a lifted, lively mood. In some embodiments, the improvement in mood is assessed relative to a baseline defined by the individual's normal, everyday mood when they have not been administered (or have not self-administered) a disclosed formulation. In some embodiments, an individual's level of stress or anxiety can be determined using known assessments and questionnaires, such as the Adjective Mood Rating Scale (AMRS; Janke and Debus, Die Eigenschaftswörterliste. Göttingen: Hogrefe; 1978) or a visual assessment scale, such as those used in empathogen psychopharmacology research (Hysek, et al. Soc Cogn Affect Neurosci. 2014; 9:1645-1652). In some embodiments, improving an individual's mood is effective for treating a mood disorder, such as affective disorder, depression, atypical depression, postpartum depression, catatonic depression, a depressive disorder due to a medical condition, seasonal affective disorder, dysthymia, substance-induced mood disorder, or a mood disorder related to another health condition.

In some embodiments, the empathogenic effect comprises an increase in energy. In some embodiments, disclosed empathogenic botanical formulations increase mental energy, or reduce mental fatigue. In some embodiments, disclosed empathogenic botanical formulations improve cognition, mental acuity, or focus. In some embodiments, increases in mental energy, reductions in mental fatigue, improvements in cognition, improvements in mental acuity, and improvements in focus, are assessed relative to a baseline defined by the individual's normal, everyday cognition when they have not been administered (or have not self-administered) a disclosed formulation.

In some embodiments, a disclosed formulation modulates neurotransmission in an individual. In some embodiments, modulating neurotransmission comprises modulating serotonergic neurotransmission (e.g., directly activating/agonizing a serotonin receptor, promoting serotonin release, inhibiting serotonin reuptake). In some embodiments, the formulation's effects on serotonergic neurotransmission cause or contribute to the empathogenic effects of the formulation. In some embodiments, modulating neurotransmission comprises inhibiting phosphodiesterase 4 (PDE4). In some embodiments, inhibiting PDE4 causes or contributes to an empathogenic and/or antidepressant effect of a disclosed formulation. In some embodiments, modulating neurotransmission comprises modulating GABAergic (e.g., binding to GABAA and/or GABA_{B}). In some embodiments, modulating GABAergic neurotransmission causes or contributes to the empathogenic effect of a disclosed formulation.

In some embodiments, a disclosed formulation is formulated to facilitate absorption through the oral mucosa. In some embodiments, absorption through the oral mucosa reduces undesirable effects of gastrointestinal absorption (e.g., gastrointestinal discomfort) that can result from swallowing a material that is rich in alkaloids and plant material.

In some embodiments, the combinations of ingredients in a disclosed formulation result in synergistic effects. “Synergistic effects” should be understood to include increases in flavor masking, flavor or taste profile, mouthfeel or mouth sensation, potency, bioactivity, bioaccessibility, bioavailability, stability or shelf life, empathogenic effect, therapeutic effect, beneficial effect, or the like, including as described elsewhere herein or as understood from the disclosure, that are greater than the additive contributions of the components acting alone.

Numerous methods known to those of skill in the art exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang, Front Pharmacol., 2019; 10:1222), or the equation of Loewe additivity (Loewe & Muischnek, Arch. Exp. Pathol Pharmacol. 1926; 114:313-326). A synergistic effect also may be calculated using methods such as the Sigmoid-Emax equation (Holford & Scheiner, Clin. Pharmacokinet. 1981; 6:429-453) and the median-effect equation (Chou & Talalay, Adv. Enzyme Regul. 1984; 22:27-55). The corresponding graphs associated with the equations referred to above are the concentration-effect curve and combination index curve, respectively. Each equation referred to above may be applied to experimental data to generate a corresponding graph to aid in assessing the effects of, e.g., the combination or mixture of kanna and/or other plant material.

In some embodiments, the inclusion of the synergistic plant composition in a disclosed formulation enhances the efficacy and bioavailability of the kanna composition. For example, the synergistic plant composition can stimulate salivary glands and the trigeminal nerve in the oral cavity, which can facilitate mastication of the empathogenic botanical formulation and promote buccal absorption of kanna alkaloids and other bioavailable compounds.

In some embodiments, the kanna composition and the synergistic plant composition comprise bioavailable and neurochemically active components that act simultaneously and synergistically to enhance the empathogenic effect of disclosed formulations.

In some embodiments, the synergistic plant composition exerts an antimicrobial or antiseptic effect on the empathogenic botanical formulation, thereby obviating the need for any added preservatives.

In some embodiments, the synergistic plant composition creates a pleasurable flavor and/or aroma sensation for the individual, thereby obviating the need for any added sugars or artificial sweeteners. These and other potential effects of the synergistic plant composition are described in further detail herein.

In some embodiments, a disclosed synergistic plant composition comprises Acmella oleracea. In some embodiments, the inclusion of Acmella oleracea in a disclosed synergistic plant composition provides an immunomodulatory effect, such as an immunostimulant effect. In some embodiments, the inclusion of Acmella oleracea in a disclosed synergistic plant composition increases salivation, aiding in the mastication and disintegration of the formulation and promoting the absorption (e.g., sublingual, mucosal, buccal, or oral absorption) of its constituents. In some embodiments, the inclusion of Acmella oleracea in a disclosed synergistic plant composition stimulates the nervous system. In some embodiments, stimulating the nervous system comprises stimulating the trigeminal nerve in the oral cavity. In some embodiments, stimulating the nervous system comprises creating a “tingling” sensation in the mouth. In some embodiments, the inclusion of Acmella oleracea in a disclosed synergistic plant composition activates ion channels in the oral cavity. In some embodiments, the inclusion of Acmella oleracea in a disclosed synergistic plant composition further provides an antimicrobial or antiseptic effect.

In some embodiments, a disclosed synergistic plant composition comprises Saussurea involucrata. In some embodiments, the inclusion of Saussurea involucrata in a disclosed synergistic plant composition provides immune-enhancing effects. In some embodiments, the inclusion of Saussurea involucrata in a disclosed synergistic plant composition provides mood-elevating effects. In some embodiments, the inclusion of Saussurea involucrata in a synergistic plant composition enhances blood circulation or blood oxygenation.

In some embodiments, a disclosed synergistic plant composition comprises Lavandula angustifolia. In some embodiments, the inclusion of Lavandula angustifolia in a disclosed synergistic plant composition provides a neurochemical effect, such as the release of serotonin.

In some embodiments, a disclosed synergistic plant composition comprises Mentha sp. In some embodiments, the inclusion of Mentha sp. in a disclosed synergistic plant composition exerts a neurochemical effect. In some embodiments, the inclusion of Mentha sp. in a disclosed synergistic plant composition eases digestion. In some embodiments, the inclusion of Mentha sp. in a disclosed synergistic plant composition provides an antiseptic or antimicrobial effect.

In some embodiments, a disclosed synergistic plant composition comprises Zingiber officinale. In some embodiments, the inclusion of Zingiber officinale in a disclosed synergistic plant composition imparts antiemetic properties to the empathogenic botanical formulation.

In some embodiments, a disclosed synergistic plant composition comprises chamomile. In some embodiments, the inclusion of chamomile in a synergistic plant composition eases the passage of disclosed empathogenic botanical formulation through the gastrointestinal tract.

In some embodiments, a disclosed synergistic plant composition comprises a sweet woodruff extract. In some embodiments, the inclusion of sweet woodruff (e.g., sweet woodruff extract) in a synergistic plant composition imparts an anti-migraine effect to a disclosed empathogenic botanical formulation.

In some embodiments, a disclosed synergistic plant composition comprises Silybum marianum. In embodiments, the inclusion of S. marianum in a synergistic plant composition imparts an additional beneficial and/or synergistic effect, such as an anti-diabetic, hepatoprotective, hypocholesterolaemic, anti-hypertensive, anti-inflammatory, anti-cancer, anti-oxidant, anti-spasmodic, neuroprotective, anti-viral, immunomodulant, cardioprotective, demulcent, or anti-haemorrhagic effect.

In some embodiments, a disclosed synergistic plant composition comprises Aspalathus linearis. In some embodiments, the inclusion of Aspalathus linearis in a disclosed synergistic plant composition improves the mental clarity or “grounded” energy of the individual after administration of a disclosed empathogenic botanical formulation.

In some embodiments, a disclosed synergistic plant composition comprises Rosa sp. In some embodiments, the Rosa sp. acts as a “head/heart connector” and/or improves the flavor profile in a disclosed formulation, improving the subjective sensory experience of consuming the formulation, and contributing to synergistic empathogenic effects.

In some embodiments, synergistic effects result from the absorption sequence of the ingredients of a disclosed formulation, which may be manipulated and determined by the ratios of ingredients, as well as the particle size of the ingredients and resulting impacts on their solubility and dissolution rate. For example, in some embodiments, the particle size of an Acmella plant powder in a disclosed formulation is between about 0.1 and 1.0 mm, which allows the Acmella powder to be rapidly dissolved by saliva and released into the oral cavity. In some embodiments, the Acmella powder stimulates salivary glands to increase salivary production, which can increase the dissolution rates of other ingredients, and consequently increase their absorption through the oral mucosa. In some embodiments, the effects of the Acmella powder synergistically increase the dissolution of Lavandula plant material in a disclosed formulation, which can provide a subliminal retro-olfactory sensation and directly activate and/or “prime” serotonin receptors for activation by the kanna extract (e.g., through the release of the kanna alkaloids contained therein). In some embodiments, the kanna extract or extracts in a disclosed formulation become dissolved after the Acmella and Lavandula plant material has created an enhanced environment for the absorption of the kanna alkaloids through mucosa in the oral cavity (and/or in other components of the gastrointestinal tract), thereby maximizing the bioavailability of the kanna alkaloids. Further, in some embodiments, the flavoring components of a disclosed formulation work synergistically to mask the bitterness of the kanna alkaloids, thereby creating a pleasurable sensory experience for an individual consuming a disclosed formulation.

In embodiments wherein a disclosed kanna composition comprises multiple sources of kanna alkaloids (e.g., a combination of kanna plant powder and one or more kanna extracts), the release profile of kanna alkaloids and other synergistic components of the formulation results in a sustained empathogenic effect in an individual who consumes a disclosed empathogenic botanical formulation. In some embodiments, the individual feels an initial “burst” of empathogenic effect. In some embodiments, the initial empathogenic effect results from rapid absorption of kanna alkaloids (e.g., mesembrine) from a concentrated (e.g., a first, high-mesembrine) extract. In some embodiments, the initial empathogenic effect comprises a sense of euphoria that is felt rapidly after consuming the formulation. In some embodiments, the initial effect is felt within 1 hour, 45 minutes, 30 minutes, 20 minutes, 10 minutes, or 5 minutes after the individual consumes the formulation. In some embodiments, the individual feels a prolonged empathogenic effect subsequent to the initial “burst” resulting from sustained release of kanna alkaloids from additional components of the kanna composition (e.g., a second, lower-mesembrine kanna extract and/or kanna plant powder). In some embodiments, the sustained empathogenic effect has a duration (e.g., of subjective experience, and/or of time during which blood or plasma concentrations of kanna alkaloids are sufficiently high to create a psychoactive effect) of at least 1 hour, 2 hours, 3 hours, 4 hours, 5 hours, 6 hours, 7 hours, 8 hours, 12 hours, 16 hours, 18 hours, or 24 hours. In some embodiments, the individual's experience of an initial effect followed by a prolonged, sustained effect results in a greater overall empathogenic effect, improved effects on health and well-being, and/or improved clinical potential for treating a medical condition, as compared to the effects resulting from administration of a conventional kanna formulation (e.g., a kanna extract or tincture).

Measures of effects include any outcome measure, endpoint, effect measure, or measure of effect which is used to assess the effect, both positive and negative, of a method, whether reported by the individual (e.g., questionnaires), based on other individual data (e.g., data monitoring), gathered through laboratory tests such as blood work, urine samples, etc., through examination by a medical or health professional or facilitator, or by digital tools or means, e.g., electronic tools such as online tools, smartphones, wireless devices, biosensors, or health apps.

In some embodiments, measuring the empathogenic effect will include an assessment. “Assessment” refers to any means or method used with an individual, whether before, during, after, or unrelated in time to a specific treatment protocol, to measure, estimate, or evaluate a nature, ability, symptom, disorder, or other characteristic of the individual, whether qualitatively or quantitatively, and whether performed by a facilitator (e.g., an interview), by the individual his or herself (e.g., a self-reported questionnaire), by a third-party or by a computer, including a device (e.g., a medical sensor or biosensor, a watch or fitness tracker, or a “wearable”), and whether graded by a human decision-maker or an artificial intelligence, machine learning, or computer algorithm.

An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment will include the use of an electronic psychiatric notes system, where relevant clinical information will be recorded for the duration of the therapy by a therapist interacting face-to-face with an individual, and will also include the use of computer systems where the therapist and patient interact virtually (either synchronously or asynchronously), as well as where an individual only interacts with a computer (“computer” broadly meaning any electronic tool suitable for such purposes, including desktop, laptop, and notebook computers; tablets, smartphones, and other mobile devices; watches, fitness trackers, and personal electronic devices; and the like).

In general, all of the disclosed formulations and methods will be appreciated to work for all individuals, although individual variation is to be expected, and will be readily appreciated. Where there is variation between individuals, modification to the disclosed methods will be understood based on the teachings herein in combination with general knowledge in the art. In some embodiments, certain personalized approaches (i.e., “personalized” or “precision” medicine) may be utilized, based on individual characteristics, such as drug metabolism—e.g., genetic variation relating to CYP enzymes, such as those responsible for the metabolism of kanna alkaloids (i.e., CYP1A2, CYP2B6, CYP2C9, CYP2C19, CYP2D6, CYP3A4, CYP3A5) (Meyer, et al. Anal. Bioanal. Chem. 2015; 407(3):761-778).

### E. METHODS OF MANUFACTURE

In another aspect, provided are methods of manufacturing the empathogenic botanical formulations.

In some embodiments, a disclosed method of manufacture comprises preparing a blend of a kanna composition, a synergistic plant composition, and a binding composition.

In embodiments, individual ingredients of the are pre-processed separately prior to incorporation into the blend. For example, in some embodiments, an individual ingredient of the blend (e.g., Acmella oleracea plant powder) is prepared by pulverizing or grinding Acmella oleracea plant material (e.g., using a blender, grinder, etc.) to obtain a desired particle size (e.g., 100 microns) for the ingredient. Pre-processing the ingredients of the blend can result in the blend having an ingredient particle size profile that releases the contained ingredients upon administration (e.g., oral administration) in a controlled, predetermined order that in some embodiments contributes to the synergistic effects of a disclosed formulation (e.g., resulting in an improvement of the flavor profile, mouthfeel, potency, bioactivity, bioaccessibility, bioavailability, stability, shelf life, empathogenic effect, therapeutic effect, or beneficial effect of the formulation).

In embodiments, preparing the blend comprises mixing the ingredients of the blend to produce a powder blend. Mixing can be conducted using any suitable means known to one of skill (e.g., a motorized or manually operated powder mixer, for instance a tumbler blender, paddle mixer, ribbon blender, etc.). The resulting powder blend can be stored prior to use in a UV-proof container (e.g., a sealed UV-proof glass jar).

In some embodiments, a disclosed method of manufacture comprises mixing the powder blend with a liquid. In some embodiments, the liquid comprises water (e.g., distilled water). In some embodiments, the liquid comprises vegetable glycerin. In some embodiments, the liquid comprises both water and vegetable glycerin. In such embodiments, the mixture can comprise water and vegetable glycerin in a water:glycerin ratio of between about 1:1 and 20:1 (on a w/w or v/v basis). In some embodiments, the water:glycerin ratio is between about 1:1 and 10:1. In some embodiments, the water:glycerin ratio is between about 10:1 and 20:1. In some embodiments, the water:glycerin ratio is between about 2:1 and 8:1. In some embodiments, the water:glycerin ratio is between about 4:1 and 7:1. In some embodiments, the water:glycerin ratio is between about 6:1 and 7:1. In some embodiments, the water:glycerin ratio is about 1:1, 2:1, 3:1, 4:1, 5:1, 6:1, 7:1, 8:1, 9:1, or 10:1, including ranges in between these ratios.

In some embodiments, the powder:liquid ratio of the powder blend and the liquid is between about 1:5 and 5:1 (on a w/w, w/v, or v/v basis). In some embodiments, the powder:liquid ratio is between about 1:4 and 4:1. In some embodiments, the powder:liquid ratio is between about 1:3 and 3:1. In some embodiments, the powder:liquid ratio is between about 1:2 and 2:1. In some embodiments, the powder:liquid ratio is about 1:1. In some embodiments, the powder:liquid ratio is about 1.1:1.

In some embodiments, mixing the powder blend and the liquid (e.g., using a motorized or manually operated mixer) results in a paste. In some embodiments, a disclosed method of manufacture comprises forming the paste into unit dosage forms (e.g., single-dose “chews”). In some embodiments, forming the paste into unit dosage forms comprises dividing the paste into multiple unit dosage forms using, for example, transferring the paste into molds (e.g., silicone molds) using suitable means therefor (e.g., a silicone piping bag with a rigid tip, such as those used in baking).

In some embodiments, the method comprises drying the paste (or unit dosage forms thereof). Drying results in a reduction of the moisture content, resulting in a final product that has a chewable, soft, and rubbery texture. Drying can be conducted using any suitable means known to one of skill (e.g., oven-drying, air-drying, placing into a dehumidified/desiccated environment). In embodiments wherein the method is conducted entirely at ambient temperature (without the use of heat), the drying is conducted by air-drying. In one example, drying is conducted in a ventilated, dark box. In some embodiments, drying can be assisted using air flow (e.g., from a fan or similar means).

Conventional methods of manufacturing often rely upon heating a formulation to promote dissolution of ingredients and/or homogenization of mixtures. Furthermore, heat may be used to induce gelation of the final product, depending on what binding ingredients are used (e.g., in certain agar-based formulations). For example, agar/water mixtures are typically heated to above 85° C. to induce gelation. Other binding agents (e.g., gelatin, starch-based binding agents) also frequently require heat in conventional methods of manufacture, e.g., temperatures in excess of 70° C. Although heat can be used during any step of a disclosed method, in some embodiments, the method is conducted entirely without applying heat. Hence, in some embodiments, no step of a disclosed method comprises heating the formulation, paste, or powder blend to a temperature above about 30° C., 40° C., 50° C., 60° C., 70° C., 80° C., or 90° C. In some embodiments, each step of the method is conducted at ambient temperature (e.g., about 20-25° C.). In some embodiments, the method does not comprise the use of a means for heating, such as a hot plate, oven, hot-water bath, oil bath, heating coil, heat exchanger, heat gun, or flame (e.g., Bunsen burner). In some embodiments, avoiding the use of heat results in a formulation with improved properties (e.g., improved sensory characteristics or bioactive properties). For example, in some embodiments, conducting the method entirely at ambient temperature results in a formulation that has a higher concentration of volatile flavor and/or aroma compounds, or improved potency of bioactive alkaloids, due to substantially reduced evaporation and/or thermal degradation of these components compared to when heat is applied.

### F. EXAMPLES

The following examples are for illustrative purposes only and not to limit the scope of the invention.

### Example 1: Blend of Kanna Composition and Synergistic Plant Composition

### Formula A:

To produce a blend of the kanna composition and the synergistic plant composition, the following ingredients were combined in the specified order, in a large stainless steel lab bowl. The kanna plant powder was an unfermented plant powder, specifically a full-spectrum kanna plant extract, but could be a fermented plant powder or a mixture of fermented and unfermented plant powders. Acmella oleracea plant powder and Lavandula angustifolia plant powder were produced by pulverizing the flowers of these plants using a high-speed grinder for 30 seconds. The particle size of the ground flowers was less than about 100 microns. The Acmella oleracea and Lavandula angustifolia plant powders were stored in UV-proof glass jars.

| | | Amount |
|---|---|---|
| Order | Ingredient | (volume %) |
| 1^{st} | Acmella oleracea plant powder | 20 |
| 2^{nd} | Lavandula angustifolia plant powder | 2 |
| 3^{rd} | Acacia senegal plant powder | 63 |
| 4^{th} | Kanna extract | 4 |
| 5^{th} | Kanna plant powder | 1 |
| 6^{th} | Additional ingredients | 10 |
| | (Saussurea involucrata plant material, | |
| | Menta spicata extract, Rosa sp. plant material, | |
| | flavoring agents) | |

The mixture was transferred by funnel into a powder mixer and mixed for 10 minutes at low rotation, then 40 minutes at high rotation. The resulting powder blend was sieved using an electric powder sifter to remove particles that were greater than 100 microns in diameter, then stored in an airtight UV-proof container.

### Formula B:

To produce a blend of the kanna composition, synergistic plant composition, and binding composition, the following ingredients were combined in a large stainless steel lab bowl. Acmella oleracea plant powder and Lavandula angustifolia plant powder were produced by pulverizing the flowers of these plants using a high-speed grinder for 30 seconds. The particle size of the ground flowers was less than about 100 microns. The Acmella oleracea plant powder and Lavandula angustifolia plant powder were stored in UV-proof glass jars.

| | Amount |
|---|---|
| Ingredient | (volume %) |
| Acacia senegal plant powder + Guar gum | 80 |
| Additional ingredients | 20 |
| (kanna extracts, kanna plant powder, | |
| Acmella oleracea plant powder, | |
| Saussurea involucrata plant material, Menta spicata extract, | |
| Lavandula angustifolia plant powder, rose petal powder, | |
| Zingiber officinale root powder, flavoring agents) | |

The mixture was transferred by funnel into a powder mixture and mixed for 10 minutes at low rotation, then 40 minutes at high rotation. The resulting powder blend was sieved using an electric powder sifter to remove particles that were greater than 100 microns in diameter, then stored in an airtight UV-proof container.

### Formula C:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is about 100 microns. The Acmella oleracea plant powder and Lavandula angustifolia plant powder are stored in UV-proof glass jars.

| | | Amount |
|---|---|---|
| Order | Ingredient | (volume %) |
| 1^{st} | Acmella oleracea plant powder | 15-25 |
| 2^{nd} | Lavandula angustifolia plant powder | 1-3 |
| 3^{rd} | Acacia senegal plant powder | 50-70 |
| 4^{th} | Kanna extract | 1-5 |
| 5^{th} | Kanna plant powder | 1-5 |
| 6^{th} | Additional ingredients | 1-10 |
| | (Saussurea involucrata plant material, | |
| | Menta spicata extract, flavoring agents) | |

The mixture is transferred by funnel into a powder mixer and mixed for 10 minutes at low rotation, then 40 minutes at high rotation. The resulting powder blend is stored in an airtight UV-proof container.

### Formula D:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is about 100 microns. The Acmella oleracea plant powder and Lavandula angustifolia plant powder are stored in UV-proof glass jars.

| | | Amount |
|---|---|---|
| Order | Ingredient | (volume %) |
| 1^{st} | Acmella oleracea plant powder | 20 |
| 2^{nd} | Lavandula angustifolia plant powder | 1 |
| 3^{rd} | Acacia senegal plant powder | 60 |
| 4^{th} | Zingiber officinale root powder | 5 |
| 5^{th} | Chamaemelum nobile plant powder | 5 |
| 6^{th} | Kanna plant powder | 3 |
| 7^{th} | Flavor ingredients | 6 |

The mixture is transferred by funnel into a powder mixer and mixed for 10 minutes at low rotation, then 40 minutes at high rotation. The resulting powder blend is stored in an airtight UV-proof container.

### Formula E:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is about 100 microns. The Acmella oleracea plant powder and Lavandula angustifolia plant powder are stored in UV-proof glass jars.

| | | Amount |
|---|---|---|
| Order | Ingredient | (volume %) |
| 1^{st} | Acmella oleracea plant powder | 15 |
| 2^{nd} | Lavandula angustifolia plant powder | 3 |
| 3^{rd} | Acacia senegal plant powder | 55 |
| 4^{th} | Kanna extract | 10 |
| 5^{th} | Chamaemelum nobile plant powder | 5 |
| 6^{th} | Mentha spicata extract | 7 |
| 7^{th} | Flavor ingredients | 5 |

The mixture is transferred by funnel into a powder mixer and mixed for 10 minutes at low rotation, then 40 minutes at high rotation. The resulting powder blend is stored in an airtight UV-proof container.

### Formula E:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is about 100 microns. The Acmella oleracea plant powder and Lavandula angustifolia plant powder are stored in UV-proof glass jars.

| | | Amount |
|---|---|---|
| Order | Ingredient | (volume %) |
| 1^{st} | Acmella oleracea plant powder | 5-40 |
| 2^{nd} | Lavandula angustifolia plant powder | 1-5 |
| 3^{rd} | Acacia senegal plant powder | 40-80 |
| 4^{th} | Kanna extract | 1-10 |
| 5^{th} | Kanna plant powder | 1-5 |
| 6^{th} | Mentha spicata extract | 1.5 |
| 7^{th} | Chamaemelum nobile plant powder | 1-5 |
| 8^{th} | Zingiber officinale root powder | 1-5 |
| 9^{th} | Saussurea involucrata plant powder | 1-5 |
| 10^{th} | Silybum marianum plant powder | 1-5 |
| 11^{th} | Aspalathus linearis powder | 1-5 |
| 12^{th} | Flavoring agents | 1-10 |

The mixture is transferred by funnel into a powder mixer and mixed for 10 minutes at low rotation, then 40 minutes at high rotation. The resulting powder blend is stored in an airtight UV-proof container.

### Formula G:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is about 100 microns. The Acmella oleracea plant powder and Lavandula angustifolia plant powder are stored in UV-proof glass jars.

| | | Amount |
|---|---|---|
| Order | Ingredient | (volume %) |
| 1^{st} | Acmella oleracea plant powder | 15 |
| 2^{nd} | Lavandula angustifolia plant powder | 2 |
| 3^{rd} | Acacia senegal plant powder | 55 |
| 4^{th} | Kanna extract | 3 |
| 5^{th} | Kanna plant powder | 3 |
| 6^{th} | Mentha spicata extract | 1.5 |
| 7^{th} | Chamaemelum nobile plant powder | 4 |
| 8^{th} | Zingiber officinale root powder | 2 |
| 9^{th} | Saussurea involucrata plant powder | 2 |
| 10^{th} | Silybum marianum plant powder | 2.5 |
| 11^{th} | Aspalathus linearis powder | 5 |
| 12^{th} | Flavoring agents | 5 |

The mixture is transferred by funnel into a powder mixer and mixed for 10 minutes at low rotation, then 40 minutes at high rotation. The resulting powder blend is stored in an airtight UV-proof container.

### Formula H:

To produce a blend of the kanna composition and the synergistic plant composition, the following ingredients were combined in the specified order, in a large stainless steel lab bowl. Acmella oleracea plant powder and Lavandula angustifolia plant powder were produced by pulverizing the flowers of these plants using a high-speed grinder for 30 seconds. The particle size of the ground flowers was about 100 microns. The Acmella oleracea and Lavandula angustifolia plant powders were stored in UV-proof glass jars.

| | | Amount |
|---|---|---|
| Order | Ingredient | (volume %) |
| 1^{st} | Acmella oleracea plant powder | 20 |
| 2^{nd} | Lavandula angustifolia plant powder | 2 |
| 3^{rd} | Acacia senegal plant powder | 63 |
| 4^{th} | Kanna extract | 4 |
| 5^{th} | Kanna plant powder | 1 |
| 6^{th} | Additional ingredients | 10 |
| | (Saussurea involucrata plant material, | |
| | Menta spicata extract, flavoring agents) | |

The mixture was transferred by funnel into a powder mixer and mixed for 10 minutes at low rotation, then 40 minutes at high rotation. The resulting powder blend was stored in an airtight UV-proof container.

### Formula G:

To produce a blend of the kanna composition and the synergistic plant composition, the following ingredients are combined in the specified order, in a large stainless steel lab bowl. Acmella oleracea plant powder and Lavandula angustifolia plant powder are produced by pulverizing the flowers of these plants using a high-speed grinder for 30 seconds. The particle size of the ground flowers is less than about 100 microns. The A. oleracea plant powder and L. angustifolia plant powder are stored in UV-proof glass jars.

| | | Amount |
|---|---|---|
| Order | Ingredient | (volume %) |
| 1^{st} | Acmella oleracea plant powder | 10 |
| 2^{nd} | Lavandula angustifolia plant powder | 1 |
| 3^{rd} | Acacia senegal plant powder | 70 |
| 4^{th} | Kanna extract | 5 |
| 5^{th} | Kanna plant powder | 5 |
| 6^{th} | Additional ingredients | 9 |
| | (Saussurea involucrata plant material, | |
| | Menta spicata extract, flavoring agents) | |

The mixture is transferred by funnel into a powder mixture and mixed for 10 minutes at low rotation, then 40 minutes at high rotation. Optionally, the resulting powder blend was sieved using an electric powder sifter to remove particles that were greater than 100 microns in diameter, then stored in an airtight UV-proof container.

### Example 2: Production of Empathogenic Botanical Formulations

The workstation was sanitized with a mix of rubbing alcohol and distilled water, and empathogenic botanical formulations were prepared according to the following steps:
1. Weighed out 35 g of the blend of kanna composition and synergistic plant composition of Example 1, Formula A (“blend of Example 1A”) into a borosilicate glass mixing bowl.
2. Added 30 g lab-grade distilled water to a 250 ml beaker.
3. Weighed 5 g vegetable glycerin into a separate beaker.
4. Poured 28 g of the distilled water into the beaker containing the 5 g of vegetable glycerin. Stirred quickly ten times to obtain a homogenous mixture.
5. Quickly and evenly combined the finished binder (water/vegetable glycerin) solution with the 35 g of kanna plant powder blend in the glass mixing bowl.
6. With an electric mixer, started mixing the binder mix with the blend of Example 1A in fast circular motions for about 30 seconds clockwise and 30 seconds counterclockwise until the mixture turned into a homogenous paste resembling moist dough.
7. Transferred the mixture into a silicone piping back with a T-shaped tip.
8. Applied medium pressure on the piping bag to fill silicone molds to maximum capacity without forming bubbles.
9. Removed residual paste from the top of the molds by scraping in a fast swiping motion until the top of the fill was flush with the top of the mold. The poured paste was not touched after this process.
10. After successfully filling the molds, they were brought to a clean lab curing rack, highly ventilated using a fan at room temperature in a closed dark box. The molds sat in the box for 48 hours and were flipped during the drying process to assist with even drying.

The finished empathogenic botanical formulations were ejected from the molds and packaged according to the following procedure (gloves, mask, and hairnet were worn during the ejection procedure, and a new set of gloves was worn while placing the finished formulations inside of the final packaging):
1. By rolling the silicone molds, each cavity was made to release the formulation it contained.
2. Gently removed each formulation, and placed it on top of a silicone matt placed inside of an airtight container for the final drying process before packaging. (Chews are not stacked until fully dried.)
3. After each formulation was successfully removed from the mold and placed inside of the airtight UV-proof container, they were placed in a cool dark environment before final storage.

Shelf-life was estimated to be >10 years, and the softness of the formulation similar to a rubber-like structure was estimated to be 4-6 years. If formulations became dehydrated, they could be rehydrated by placing them into a lab-grade humidor.

### Example 3: Demonstration of Synergy or Modulation of Neurotransmission by Formulations

The ability of disclosed empathogenic botanical formulations to provide one or more synergies and/or to modulate neurotransmission in a mammal, such as a human, and thereby produce an empathogenic effect as described herein, can be assessed using a variety of techniques known to those of skill in the art, including in vitro, ex vivo, and in vivo techniques. The effects of disclosed empathogenic botanical formulations can be assessed relative to a comparator formulation. The comparator formulation can be a kanna composition (such as kanna plant material, or a kanna extract). The comparator can be a plant composition (such as Acacia senegal plant material, Acmella oleracea plant material, Saussurea involucrata plant material, Lavandula angustifolia plant material, Mentha spicata extract, Zingiber officinale plant material, Chamaemelum nobile plant material, sweet woodruff extract, Silybum marianum plant material, or Aspalathus linearis plant material) assessed in isolation or in combination with a kanna composition. Results of such assessments can show synergistic effects of the combination of the kanna composition and the synergistic plant composition in disclosed empathogenic botanical formulations. Such synergistic effects include, but not limited to: increases in flavor masking, flavor or taste profile, mouthfeel or mouth sensation, potency, bioactivity, bioaccessibility, bioavailability, stability or shelf life, empathogenic effect, therapeutic effect, beneficial effect, or the like, as will be understood by one of skill, and as described in various embodiments herein. Results can also show that such synergistic effects also include modulating neurotransmission in a manner that produces, or is likely to produce, empathogenic effects in an individual who has been administered (or who has self-administered) a disclosed formulation.

### F. EXEMPLARY ASPECTS AND EMBODIMENTS

Among the various aspects and embodiments of the disclosure are the following, which will be understood to be exemplary and not limiting.

In a first exemplary aspect, provided are empathogenic botanical formulations, comprising:
(i) a kanna composition; (ii) a synergistic plant composition; and (iii) a binding composition.

In embodiments, the kanna composition comprises a kanna extract. In embodiments, the kanna composition comprises kanna plant material. In embodiments, the kanna plant material is a fermented kanna plant powder. In embodiments, the kanna plant material is an unfermented kanna plant powder. In embodiments, the kanna plant material is a mixture of one or more fermented kanna plant powders and one or more unfermented kanna plant powders. In embodiments, the kanna composition comprises a kanna extract and kanna plant powder. In embodiments, the kanna plant powder has a particle size of less than about 75 microns. In embodiments, the synergistic plant composition increases the flavor profile, mouthfeel, potency, bioactivity, bioaccessibility, bioavailability, stability, shelf life, empathogenic effect, therapeutic effect, or beneficial effect of the formulation. In embodiments, the synergistic plant composition comprises Acacia senegal plant material. In embodiments, the synergistic plant composition comprises between about 40% and 80%, or between about 50% and 70% (v/v) of Acacia senegal plant material. In embodiments, the Acacia senegal plant material is Acacia senegal plant powder. In embodiments, the Acacia senegal plant powder has a particle size of about 0.5 mm.

In embodiments, the synergistic plant composition comprises Acmella oleracea plant material. In embodiments, the synergistic plant composition comprises between about 5% and 40%, between about 10% and 30%, or between about 15% and 25% (v/v) of Acmella oleracea plant material. In embodiments, the Acmella oleracea plant material is Acmella oleracea plant powder. In embodiments, the Acmella oleracea plant powder has a particle size of about 100 microns. In embodiments, the synergistic plant composition comprises Saussurea involucrata plant material. In embodiments, the synergistic plant composition comprises between about 1% and 3% (v/v) of Saussurea involucrata plant material. In embodiments, the Saussurea involucrata plant material is Saussurea involucrata plant powder. In embodiments, the Saussurea involucrata plant powder has a particle size of about 1 mm.

In embodiments, the synergistic plant composition comprises Lavandula angustifolia plant material. In embodiments, the synergistic plant composition comprises between about 0.1% and 5%, or between about 1% and 3% (v/v) of Lavandula angustifolia plant material. In embodiments, the Lavandula angustifolia plant material is Lavandula angustifolia plant powder. In embodiments, the Lavandula angustifolia plant powder has a particle size of about 100 microns. In embodiments, the synergistic plant composition comprises Mentha spicata plant material. In embodiments, the Mentha spicata plant material is Mentha spicata extract. In embodiments, the synergistic plant composition comprises between about 1% and 3% (v/v) of Mentha spicata extract. In embodiments, the synergistic plant composition comprises one or more of: Zingiber officinale plant material, Chamaemelum nobile plant material, sweet woodruff extract, Silybum marianum plant material, and Aspalathus linearis plant material.

In embodiments, the synergistic plant composition comprises a flavoring agent. In embodiments, the flavoring agent is an organic or natural flavor, a sweet flavor, a fruit flavor, a berry flavor, or a combination thereof. In embodiments, the synergistic plant composition comprises Acacia senegal plant material, Acmella oleracea plant material, Saussurea involucrata plant material, Lavandula angustifolia plant material, and Mentha spicata extract, and at least one flavoring agent. In embodiments, the synergistic plant composition comprises Acacia senegal plant powder, Acmella oleracea plant powder, Saussurea involucrata plant powder, Lavandula angustifolia plant powder, and Mentha spicata extract, and at least one flavoring agent.

In embodiments, the binding composition comprises agar. In embodiments, the binding composition comprises vegetable glycerin. In embodiments, the binding composition comprises distilled water. In embodiments, the binding composition comprises agar, vegetable glycerin, and distilled water.

In embodiments, the empathogenic botanical formulation comprises: (a) a kanna composition comprising: a kanna extract; and fermented kanna plant powder; (b) a synergistic plant composition; (c) a binding composition; and (d) at least one flavoring agent. In embodiments, the empathogenic botanical formulation comprises: (a) a kanna composition; (b) a synergistic plant composition comprising: Acacia senegal plant powder; Acmella oleracea plant powder; Saussurea involucrata plant powder; Lavandula angustifolia plant powder; and Mentha spicata extract; (c) a binding composition; and (d) at least one flavoring agent. In embodiments, the empathogenic botanical formulation comprises: (a) a kanna composition; (b) a synergistic plant composition; and (c) a binding composition comprising: agar; vegetable glycerin; distilled water; and (d) at least one flavoring agent. In embodiments, the empathogenic botanical formulation comprises: (a) a kanna composition comprising: a kanna extract; and fermented kanna plant powder; (b) a synergistic plant composition comprising: Acacia senegal plant powder; Acmella oleracea plant powder; Saussurea involucrata plant powder; Lavandula angustifolia plant powder; and Mentha spicata extract; and (c) a binding composition comprising: agar; vegetable glycerin; distilled water; and (d) at least one flavoring agent.

In embodiments, the formulation does not contain an added preservative. In embodiments, the formulation does not contain added sugar. In embodiments, the formulation does not contain an artificial sweetener. In embodiments, the formulation does not contain a synthetic polymer. In embodiments, the formulation does not contain an added antioxidant. In embodiments, the formulation does not contain a plastic. In embodiments, the formulation does not contain an artificial flavor. In embodiments, the formulation does not contain a genetically modified organism. In embodiments, the formulation does not contain soy. In embodiments, the formulation does not contain an animal product. In embodiments, the formulation does not contain caffeine. In embodiments, the formulation does not contain a nut.

In another exemplary aspect, provided is the formulation of any of the preceding embodiments, for use in modulating neurotransmission in an individual. In another exemplary aspect, provided is a method of modulating neurotransmission in an individual, comprising administering to the mammal an effective amount of the formulation of any of the preceding embodiments. In embodiments, the neurotransmission is serotonergic neurotransmission. In embodiments, modulating neurotransmission comprises one or more of: stimulating release of serotonin, and/or reducing serotonin uptake by inhibiting the function of a serotonin transporter (SERT). In embodiments, modulating neurotransmission treats a medical condition. In another exemplary aspect, provided is a method of treating a medical condition in a subject in need of such treatment, the method comprising administering the formulation of any one of the preceding embodiments. Also provided is the use of the formulation of any of the preceding embodiments in the manufacture of a medicament for treating a medical condition.

In embodiments, the medical condition is a mental health disorder. In embodiments, the mental health disorder is selected from the group consisting of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders. In embodiments, the mental health disorder is anxiety, depression, addiction, an eating disorder, an alcohol or drug abuse or dependence disorder, OCD, or PTSD. In embodiments, depression is major depressive disorder (MDD) or treatment-resistant depression (TRD). In embodiments, anxiety is generalized anxiety disorder (GAD) or menopause-related anxiety. In embodiments, the substance use disorder is any of alcohol use disorder, nicotine dependency, opioid use disorder, sedative, hypnotic, or anxiolytic use disorder, stimulant use disorder, or tobacco use disorder. In embodiments, the medical condition is dementia or a neurodegenerative disease such as neurodegenerative dementia, Alzheimer's disease, or Parkinson's disease. In embodiments, the medical condition is a sleep-related condition, such as insomnia.

In embodiments, the formulation is administered together with one or more therapeutically beneficial activities. In embodiments, it is administered together with one or more sessions of psychotherapy.

The foregoing description, for purposes of explanation, uses specific nomenclature to provide a thorough understanding of the invention. However, it will be apparent to one in the art that specific details are not required in order to practice the invention. Thus, the foregoing disclosure of specific embodiments of the invention is for purposes of illustration and description. It is not intended to be exhaustive or to limit the invention to the precise compositions, formulations, methods, or the like disclosed; many modifications and variations are possible in view of the above teachings. The embodiments were chosen and described in order to best explain the principles of the invention and its practical applications, through the elucidation of specific examples, and to thus enable others in the art to best utilize the invention and embodiments with various modifications as suited to the particular use contemplated, when such uses are beyond the specific examples disclosed. The scope of the invention will be defined solely by the claims and their equivalents.

The invention claimed is:

1. An empathogenic botanical formulation, comprising:
i. a kanna composition comprising:
a. a first kanna extract;
b. a second kanna extract; and
c. a kanna plant powder;
ii. a synergistic plant composition; and
iii. a binding composition.

2. The formulation of claim 1, wherein the kanna plant powder is an unfermented kanna plant powder, a fermented kanna plant powder, or a mixture thereof.

3. The formulation of claim 1, wherein the kanna plant powder has a particle size of about 75 microns.

4. The formulation of claim 1, wherein the first kanna extract comprises greater than about 7% (w/w) of mesembrine.

5. The formulation of claim 4, wherein the first kanna extract comprises between about 7% and about 9% (w/w) of mesembrine.

6. The formulation of claim 1, wherein the second kanna extract comprises less than about 5% (w/w) of mesembrine.

7. The formulation of claim 1, wherein the second kanna extract comprises between about 2% and about 3% (w/w) of mesembrine.

8. The formulation of claim 1, comprising the first kanna extract and the second kanna extract in a weight ratio of between about 1:1 and about 5:1.

9. The formulation of claim 8, comprising the first kanna extract and the second kanna extract in a weight ratio of about 2:1.

10. The formulation of claim 1, wherein the weight ratio of the kanna plant powder and the total weight of the first and second kanna extracts is between about 1:1 and about 1:5.

11. The formulation of claim 10, wherein the weight ratio of the kanna plant powder and the total weight of the first and second kanna extracts is about 1:3.

12. The formulation of claim 1, wherein the synergistic plant composition comprises Acacia senegal plant material.

13. The formulation of claim 12, wherein the synergistic plant composition comprises between about 40% and about 80% (v/v) of Acacia senegal plant material.

14. The formulation of claim 12, wherein the Acacia senegal plant material is Acacia senegal plant powder.

15. The formulation of claim 14, wherein the Acacia senegal plant powder has a particle size of about 0.5 mm.

16. The formulation of claim 1, wherein the synergistic plant composition comprises Acmella oleracea plant material.

17. The formulation of claim 16, wherein the synergistic plant composition comprises between about 5% and about 40% (v/v) of Acmella oleracea plant material.

18. The formulation of claim 17, wherein the Acmella oleracea plant material is Acmella oleracea plant powder.

19. The formulation of claim 18, wherein the Acmella oleracea plant powder has a particle size of about 100 microns.

20. The formulation of claim 1, wherein the synergistic plant composition comprises Saussurea involucrata plant material.

21. The formulation of claim 20, wherein the synergistic plant composition comprises between about 1% and about 3% (v/v) of Saussurea involucrata plant material.

22. The formulation of claim 20, wherein the Saussurea involucrata plant material is Saussurea involucrata plant powder.

23. The formulation of claim 22, wherein the Saussurea involucrata plant powder has a particle size of about 1 mm.

24. The formulation of claim 1, wherein the synergistic plant composition comprises Lavandula angustifolia plant material.

25. The formulation of claim 24, wherein the synergistic plant composition comprises between about 0.1% and about 5% (v/v) of Lavandula angustifolia plant material.

26. The formulation of claim 24, wherein the Lavandula angustifolia plant material is Lavandula angustifolia plant powder.

27. The formulation of claim 26, wherein the Lavandula angustifolia plant powder has a particle size of about 100 microns.

28. The formulation of claim 1, wherein the synergistic plant composition comprises Mentha spicata plant material.

29. The formulation of claim 28, wherein the Mentha spicata plant material is Mentha spicata extract.

30. The formulation of claim 29, wherein the synergistic plant composition comprises between about 1% and about 3% (v/v) of Mentha spicata extract.

31. The formulation of claim 1, wherein the synergistic plant composition comprises Zingiber officinale plant material.

32. The formulation of claim 1, wherein the synergistic plant composition comprises Rosa sp. plant material.

33. The formulation of claim 1, wherein the synergistic plant composition comprises a combination of plant materials selected from the group of plant materials consisting of Acacia senegal plant material, Acmella oleracea plant material, Saussurea involucrata plant material, Lavandula angustifolia plant material, Mentha spicata extract, Zingiber officinale plant material, and Rosa sp. plant material.

34. The formulation of claim 33, wherein the combination of plant materials comprises two or more plant materials selected from said group of plant materials.

35. The formulation of claim 33, wherein the combination of plant materials comprises three or more plant materials selected from said group of plant materials.

36. The formulation of claim 33, wherein the combination of plant materials comprises four or more plant materials selected from said group of plant materials.

37. The formulation of claim 33, wherein the combination of plant materials comprises five or more plant materials selected from said group of plant materials.

38. The formulation of claim 33, wherein the combination of plant materials comprises six or more plant materials selected from said group of plant materials.

39. The formulation of claim 1, wherein the synergistic plant composition comprises: Acacia senegal plant material, Acmella oleracea plant material, Saussurea involucrata plant material, Lavandula angustifolia plant material, Mentha spicata extract, Zingiber officinale plant material, and Rosa sp. plant material.

40. The formulation of claim 1, wherein the binding composition comprises guar gum.

41. The formulation of claim 1, wherein the binding composition comprises vegetable glycerin.

42. The formulation of claim 1, wherein the binding composition comprises distilled water.

43. The formulation of claim 1, wherein the binding composition comprises distilled water and vegetable glycerin in a weight ratio of between about 1:1 and about 20:1.

44. The formulation of claim 43, wherein the water:glycerin weight ratio is between about 4:1 and about 7:1.

45. The formulation of claim 1, wherein the binding composition comprises guar gum, vegetable glycerin, and distilled water.

46. The formulation of claim 1, further comprising a flavoring agent.

47. The formulation of claim 46, wherein the flavoring agent is an organic or natural flavor, a sweet flavor, a fruit flavor, a berry flavor, or a combination thereof.

48. The formulation of claim 1, further comprising a coloring agent.

49. The formulation of claim 48, wherein the coloring agent is a vegetable extract.

50. The formulation of claim 49, wherein the vegetable extract comprises an extract of any of spinach, blueberry, carrot, beet, red cabbage, turmeric, annatto seed, saffron, paprika, tomato, grape, cherry, black carrot, pumpkin, sweet potato, elderberry, spirulina, bell pepper, sweet potato, kale, hibiscus flower, matcha, parsley, mint, pea, cucumber, aronia, blackberry, raspberry, cranberry, and a combination thereof.

51. An empathogenic botanical formulation, comprising:
i. a kanna composition comprising:
a. a first kanna extract;
b. a second kanna extract; and
c. a kanna plant powder;
ii. a synergistic plant composition;
iii. a binding composition;
iv. at least one flavoring agent; and
v. at least one coloring agent.

52. An empathogenic botanical formulation, comprising:
i. a kanna composition comprising:
a. a first kanna extract;
b. a second kanna extract; and
c. a kanna plant powder; and
ii. a synergistic plant composition comprising:
a. Acacia senegal plant powder;
b. Acmella oleracea plant powder; and
c. any one or more of Acacia senegal plant material, Acmella oleracea plant material, Saussurea involucrata plant material, Lavandula angustifolia plant material, Mentha spicata extract, Zingiber officinale plant material, and Rosa sp. plant material.

53. An empathogenic botanical formulation, comprising:
i. a kanna composition comprising:
a. a first kanna extract;
b. a second kanna extract; and
c. a kanna plant powder;
ii. a synergistic plant composition; and
iii. a binding composition comprising:
a. guar gum;
b. vegetable glycerin; and
c. distilled water.

54. An empathogenic botanical formulation, comprising:
i. a kanna composition comprising:
a. a first kanna extract;
b. a second kanna extract; and
c. a kanna plant powder;
ii. a synergistic plant composition comprising:
a. Acacia senegal plant powder;
b. Acmella oleracea plant powder; and
c. any one or more of Acacia senegal plant material, Acmella oleracea plant material, Saussurea involucrata plant material, Lavandula angustifolia plant material, Mentha spicata extract, Zingiber officinale plant material, and Rosa sp. plant material; and
iii. a binding composition comprising:
a. guar gum;
b. vegetable glycerin; and
c. distilled water.

55. The formulation of claim 1, wherein the formulation does not contain an added preservative.

56. The formulation of claim 1, wherein the formulation does not contain added sugar.

57. The formulation of claim 1, wherein the formulation does not contain an artificial sweetener.

58. The formulation of claim 1, wherein the formulation does not contain a synthetic polymer.

59. The formulation of claim 1, wherein the formulation does not contain an added antioxidant.

60. The formulation of claim 1, wherein the formulation does not comprise agar.

61. The formulation of claim 1, in unit dosage form.

62. The formulation of any one of claims 1-61, comprising at least one non-naturally occurring substance.

63. An empathogenic botanical formulation, such as the formulation of any one of claims 1-62, comprising:
i. a kanna composition;
ii. a synergistic plant composition; and
iii. a binding composition.

64. A method of manufacturing the empathogenic botanical formulation of any one of claims 1-61, comprising:
i. preparing a powder blend of the kanna composition, the synergistic plant composition, and the binding composition;
ii. mixing the powder blend with a liquid to obtain a paste; and
iii. drying the paste to obtain the empathogenic botanical formulation.

65. The method of claim 64, wherein the liquid comprises distilled water.

66. The method of claim 64, wherein the liquid comprises vegetable glycerin.

67. The method of claim 64, wherein the liquid comprises distilled water and vegetable glycerin.

68. The method of claim 64, wherein the liquid comprises distilled water and vegetable glycerin in a water:glycerin weight ratio of between about 1:1 and about 20:1.

69. The method of claim 68, wherein the liquid comprises distilled water and vegetable glycerin in a water:glycerin weight ratio of between about 4:1 and about 7:1.

70. The method of claim 64, further comprising dividing the paste into unit doses prior to drying the paste.

71. The method of claim 64, wherein the method does not comprise heating the powder blend, the paste, or the empathogenic botanical formulation.

72. The method of claim 64, wherein each step of the method is conducted at ambient temperature.

73. The formulation of any one of claims 1-61, for use in modulating neurotransmission in an individual.

74. Use of the formulation of any one of claims 1-61 for modulating neurotransmission in an individual.

75. A method of modulating neurotransmission in an individual, comprising administering to the individual the formulation of any one of claims 1-61.

76. The method of claim 75, wherein the neurotransmission is serotonergic neurotransmission.

77. The method of claim 76, wherein modulating neurotransmission comprises any one or more of: activating a serotonin receptor, stimulating release of serotonin, and reducing serotonin uptake by inhibiting the function of a serotonin transporter (SERT).

78. The method of claim 75, wherein the neurotransmission is GABAergic neurotransmission.

79. The method of claim 75, wherein modulating neurotransmission comprises inhibiting phosphodiesterase 4.

80. The method of claim 75, wherein modulating neurotransmission results in an empathogenic effect.

81. The method of claim 75, wherein modulating neurotransmission treats a medical condition.

82. The formulation of any one of claims 1-61, for use in producing an empathogenic effect in an individual.

83. Use of the formulation of any one of claims 1-61 for producing an empathogenic effect in an individual.

84. A method of producing an empathogenic effect in an individual, comprising administering to the individual the formulation of any one of claims 1-61.

85. The method of claim 84, wherein the empathogenic effect comprises any one or more of reducing stress, reducing anxiety, improving mood, increasing energy, reducing fatigue, creating a feeling of connection with self and others, and improving general mental health and well-being; or comprises feelings of any one or more of: deeper connection with oneself and others; restoration and coherence of body, heart, mind and spirit; head-heart harmony; being in balance and healthy homeostasis; joy and the highest form of well being; the fullness of one's aliveness; radiating from within; the ability to positively affect everything around oneself; elevated vibes; and aliveness and lifted mood.

86. The formulation of any one of claims 1-61, for use in treating a medical condition in a subject in need of such treatment.

87. Use of the formulation of any one of claims 1-61 in the manufacture of a medicament for treating a medical condition.

88. A method of treating a medical condition in a subject in need of such treatment, comprising administering to the subject the formulation of any one of claims 1-61.

89. The method of claim 88, wherein the medical condition is a mental health disorder.

90. The method of claim 89, wherein the mental health disorder is selected from the group consisting of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

91. The method of claim 89, wherein the mental health disorder is anxiety, depression, addiction, an eating disorder, an alcohol or drug abuse or dependence disorder, OCD, or PTSD.

92. The method of claim 91, wherein depression is major depressive disorder (MDD) or treatment-resistant depression (TRD).

93. The method of claim 91, wherein anxiety is generalized anxiety disorder (GAD) or menopause-related anxiety.

94. The method of claim 88, wherein the formulation is administered together with one or more therapeutically beneficial activities.

95. The method of claim 88, wherein the formulation is administered together with one or more sessions of psychotherapy.

96. The formulation manufactured according to the method of any one of claims 64-72, for use in modulating neurotransmission in an individual.

97. Use of the formulation manufactured according to the method of any one of claims 64-72 for modulating neurotransmission in an individual.

98. A method of modulating neurotransmission in an individual, comprising administering to the individual the formulation manufactured according to the method of any one of claims 64-72.

99. The formulation manufactured according to the method of any one of claims 64-72, for use in producing an empathogenic effect in an individual.

100. Use of the formulation manufactured according to the method of any one of claims 64-72 for producing an empathogenic effect in an individual.

101. A method of producing an empathogenic effect in an individual, comprising administering to the individual the formulation manufactured according to the method of any one of claims 64-72.

102. The formulation manufactured according to the method of any one of claims 64-72, for use in treating a medical condition in a subject in need of such treatment.

103. Use of the formulation manufactured according to the method of any one of claims 64-72 in the manufacture of a medicament for treating a medical condition.

104. A method of treating a medical condition in a subject in need of such treatment, comprising administering to the subject the formulation manufactured according to the method of any one of claims 64-72.

105. The formulation of claim 63, for use in modulating neurotransmission in an individual.

106. Use of the formulation of claim 63 for modulating neurotransmission in an individual.

107. A method of modulating neurotransmission in an individual, comprising administering to the individual the formulation of claim 63.

108. The formulation of claim 63, for use in producing an empathogenic effect in an individual.

109. Use of the formulation of claim 63 for producing an empathogenic effect in an individual.

110. A method of producing an empathogenic effect in an individual, comprising administering to the individual the formulation of claim 63.

111. The formulation of claim 63, for use in treating a medical condition in a subject in need of such treatment.

112. Use of the formulation of claim 63 in the manufacture of a medicament for treating a medical condition.

113. A method of treating a medical condition in a subject in need of such treatment, comprising administering to the subject the formulation of claim 63.
